(19) United States
(12) Patent Application Publication
(10) Pub. No.: US 2016/0038505 A1
(43) Pub. Date: Feb. 11, 2016

(54) METHODS AND COMPOSITIONS FOR TREATING IMPULSE CONTROL DISORDER, ANXIETY-RELATED DISORDERS, VIOLENCE AND/OR ANGER, OR REGULATING FOOD INTAKE

(71) Applicant: DemeRx, Inc., Fort Lauderdale, FL (US)

(72) Inventors: Emeline Maillet, Miami Beach, FL (US); Holger Weis, Fort Lauderdale, FL (US)

(21) Appl. No.: 14/624,415

(22) Filed: Feb. 17, 2015

Related U.S. Application Data
(60) Provisional application No. 62/033,538, filed on Aug. 5, 2014.

(51) Int. Cl. A61K 31/55

(52) U.S. Cl. CPC A61K 31/55

(57) ABSTRACT

This invention provides a method for treating anxiety-related disorder or impulse control disorder, regulating food intake, attenuating food cravings, or treating anger and/or violence and disorders associated therewith in a patient, comprising administering to the patient in need thereof a therapeutically effective amount of noribogaine, noribogaine derivative, or a pharmaceutically acceptable salt and/or solvate thereof.

### CROSS-REFERENCE TO RELATED APPLICATIONS

This application claims priority to U.S. Provisional Application No. 62/033,538, filed Aug. 5, 2014, which is incorporated herein by reference in its entirety.

### FIELD OF THE INVENTION

This invention relates generally to methods for the treatment of anxiety-related disorders, including obsessive-compulsive disorder (OCD), or impulse control disorder by administering noribogaine, a noribogaine derivative, or a pharmaceutically acceptable salt and/or solvate thereof. This invention further relates generally to methods and compositions for the regulation of food intake by administering noribogaine, a noribogaine derivative, or a pharmaceutically acceptable salt thereof. This invention further relates generally to methods and compositions for treating anger and/or violence and disorders associated therewith by administering noribogaine, a noribogaine derivative, or a pharmaceutically acceptable salt thereof.

### STATE OF THE ART

Noribogaine is a well-known member of the ibogaine family of alkaloids and is sometimes referred to as 12-hydroxyibogaine. U.S. Pat. No. 2,813,873 claims noribogaine, albeit as “12-O-demethylibogaine,” while providing an incorrect structural formula for ibogaine. The structure of noribogaine has now been thoroughly evaluated and is found to combine the features of tryptamine, tetrahydrohavaine and indolazepines. Noribogaine can be depicted by the following formula:

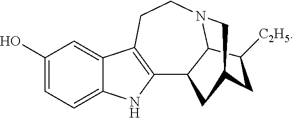

Obsessive compulsive disorder is characterized by recurrent and persistent ideas, thoughts, impulses or images (obsessions) that are ego-dystonic and/or repetitive, purposeful and intentional behaviors (compulsions) that are recognized by the person as excessive or unreasonable (American Psychiatric Association, 1994a). The obsessions or compulsions cause marked distress, are time-consuming, and/or significantly interfere with social or occupational functioning.

Panic disorder is characterized by recurrent unexpected panic attacks and associated concern about having additional attacks, worry about the implications or consequences of the attacks, and/or a significant change in behavior related to the attacks (American Psychiatric Association, 1994a). A panic attack is defined as a discrete period of intense fear or discomfort in which four (or more) of the following symptoms develop abruptly and reach a peak within 10 minutes: (1) palpitations, pounding heart, or accelerated heart rate; (2) sweating; (3) trembling or shaking; (4) sensations of shortness of breath or smothering; (5) feeling of choking; (6) chest pain or discomfort; (7) nausea or abdominal distress; (8) feeling dizzy, unsteady, lightheaded, or faint; (9) derealization (feelings of unreality) or depersonalization (being detached from oneself); fear of losing control; (11) fear of dying; (12) paresthesias (numbness or tingling sensations); (13) chills or hot flushes. Panic disorder may or may not be associated with agoraphobia, or an irrational and often disabling fear of being out in public.

Social anxiety disorder, also known as social phobia, is characterized by a marked and persistent fear of one or more social or performance situations in which the person is exposed to unfamiliar people or to possible scrutiny by others (American Psychiatric Association, 1994a). Exposure to the feared situation almost invariably provokes anxiety, which may approach the intensity of a panic attack. The feared situations are avoided or endured with intense anxiety or distress. The avoidance, anxious anticipation, or distress in the feared situation(s) interferes significantly with the person's normal routine, occupational or academic functioning, or social activities or relationships, or there is marked distress about having the phobias. Lesser degrees of performance anxiety or shyness generally do not require psychopharmacological treatment.

Generalized anxiety disorder is characterized by excessive anxiety and worry (apprehensive expectation) that is persistent for at least 6 months and which the person finds difficult to control (American Psychiatric Association, 1994a. It must be associated with at least 3 of the following 6 symptoms: restlessness or feeling keyed up or on edge, being easily fatigued, difficulty concentrating or mind going blank, irritability, muscle tension, sleep disturbance. The diagnostic criteria for this disorder are described in further detail in DSM-IV, which is incorporated herein by reference (American Psychiatric Association, 1994a).

Impulse control disorder is a class of psychiatric disorders involving the failure to resist a temptation, urge, or impulse (impulsivity) where such impulse is potentially harmful to the patient and/or others. The American Psychiatric Association's DSM-5 (May 2013) includes impulse control disorders “characterized by problems in emotional and behavioral self-control”. These include borderline personality disorder, conduct disorder, antisocial personality disorder, attention deficit hyperactivity disorder (ADHD), schizophrenia, mood disorders, pathological gambling, pyromania, intermittent explosive disorder, kleptomania, sexual compulsion, paraphilia, internet addiction, trichotillomania, pathological skin picking, and compulsive shopping. Impulse control disorder may be related to anxiety disorder and/or OCD.

Violence and anger, particularly when out of proportion to a stimulus and/or a result of pathological anger, are associated with a number of mental disorders. These include oppositional defiant disorder, attention-deficit/hyperactivity disorder and conduct disorder (in children and adolescents), psychotic disorder, bipolar disorder, antisocial, borderline, paranoid and narcissistic personality disorders, adjustment disorder with disturbance of conduct, and intermittent explosive disorder. Pathological anger and violence account for a significant portion of violent crimes, including many high-profile crimes involving multiple victims. Highly volatile individuals are over-represented in the prison system in the United States.

Over ⅔ of adults in the U.S. are overweight, with about half of those being obese. The U.S. weight loss market is estimated to be worth over $60 billion; diet pills alone account for around $1 billion. However, many diet pills contain ingredients that are at best of dubious efficacy and at worst dangerous. Obesity greatly increases a person's risk for a variety of diseases, including coronary heart disease, high blood pressure, stroke, type 2 diabetes, abnormal levels of blood fats, metabolic syndrome, cancer, osteoarthritis, sleep apnea, reproductive issues, and gallstones.

Given the prevalence and impact of anxiety disorders, impulse control disorder, anger/violence-related disorders, and overweight/obesity, there is a need for treatments that address these issues. Prior to the embodiments described herein, the therapeutic dosing of noribogaine and its derivatives for treating anxiety disorders, impulse control disorder, anger/violence-related disorders, or regulation of food intake in humans at an acceptable QT interval prolongation has not previously been addressed, especially as it relates to dosing protocols that are effective, as well as safe.

### SUMMARY OF THE INVENTION

There are certain properties of noribogaine that present this compound as a very attractive candidate for the treatment of anxiety disorders, impulse control disorder, anger/violence-related disorders, or regulation of food intake. These include the interaction of noribogaine with a variety of receptors in the brain, including nicotinic acetylcholine receptors (nAChRs) and opioid receptors (e.g., μ-opiod receptors). Further, noribogaine elevates brain serotonin levels by blocking synaptic reuptake via the SERT transporter. As such, this invention relates to methods of treating anxiety disorders, impulse control disorder, anger/violence-related disorders, or symptoms thereof, or regulation of food intake, comprising administering to a patient noribogaine, a noribogaine derivative, or a pharmaceutically acceptable salt and/or solvate thereof.

Moreover, the use of noribogaine imparts a dose dependent prolongation of the treated patient's QT interval, rendering higher dosing of noribogaine unacceptable. A prolonged QT interval is a marker of potential Torsades de Pointes, a serious arrhythmia that can result in death.

The current invention is predicated on the surprising discovery that treatment with a narrow dosage range of noribogaine or pharmaceutically acceptable salt and/or solvate thereof, between greater than about 1 mg/kg body weight and about 4 mg/kg body weight, provides a therapeutic reduction in symptoms of anxiety disorders, impulse control disorder, anger/violence-related disorders in affected patients, or provides a therapeutic reduction in food consumption. Preferably, the dose range that provide both therapeutic results and an acceptable QT interval prolongation of less than about 50 milliseconds is between about 1.3 mg per kg body weight and no more than about 4 mg per kg body weight and, more preferably between about 1 mg per kg body weight and no more than about 3 mg per kg body weight, or any subrange or subvalue within the aforementioned ranges.

In some embodiments, the dose that provides both therapeutic results and an acceptable QT interval prolongation of less than about 50 milliseconds is between about 60 mg and about 150 mg. In some embodiments, the dose that provides both therapeutic results and an acceptable QT interval prolongation of less than about 50 milliseconds is about 100 mg. In some embodiments, the dose that provides both therapeutic results and an acceptable QT interval prolongation of less than about 50 milliseconds is about 120 mg. In some embodiments, the dose that provides both therapeutic results and an acceptable QT interval prolongation of less than about 50 milliseconds is about 1.5 mg/kg body weight. In some embodiments, the dose that provides both therapeutic results and an acceptable QT interval prolongation of less than about 50 milliseconds is about 2 mg/kg body weight.

In some embodiments, the patient is administered an initial dose of noribogaine, noribogaine derivative, or pharmaceutically acceptable salt or solvate thereof, followed by one or more additional doses. In one embodiment, the initial dose is from about 50 mg to about 120 mg. In one embodiment, the one or more additional doses are lower than the initial dose. In one embodiment, the one or more additional doses are from about 5 mg to about 50 mg. In one embodiment, such a dosing regimen provides an average serum concentration of noribogaine of about 50 ng/mL to about 180 ng/mL. In one embodiment, the one or more additional doses maintain an average serum concentration of about 50 ng/mL to about 180 ng/mL over a period of time. In one embodiment, the one or more additional doses are administered periodically.

Furthermore, at very low doses, direct blood stream delivery of noribogaine may reduce symptoms of anxiety disorders, impulse control disorder, anger/violence-related disorders, or provide regulation of food intake. Such dosing is well below that previously described. Direct blood stream delivery of noribogaine enhances the amount of noribogaine delivered to the brain, because noribogaine does not pass through the liver as it does when ingested. Direct blood stream delivery of noribogaine includes sublingual, pulmonary and intranasal delivery where the noribogaine is absorbed directly into the blood stream and then into the brain. The rapid delivery of noribogaine into the brain, e.g. less than about 15 minutes, may cause a significant reduction in symptoms of anxiety disorders, impulse control disorder, anger/violence-related disorders, or food cravings.

In one aspect, this invention relates to treating anxiety disorders, impulse control disorder, anger/violence-related disorders, or regulation of food intake in a patient in need thereof comprising administering to the patient a therapeutically effective amount of noribogaine, noribogaine derivative, solvate, or pharmaceutically acceptable salt and/or solvate thereof. In one embodiment, this invention treats an anxiety disorder. In one embodiment, this invention treats OCD. In one embodiment, this invention treats generalized anxiety disorder. In one embodiment, this invention treats social anxiety disorder. In one embodiment, this invention treats panic disorder. In another embodiment, this invention treats impulse control disorder. In another embodiment, this invention treats pathological anger and/or violence. In another embodiment, this invention treats anger/violence-related disorders. In another embodiment, this invention reduces pathological anger in a patient. In another embodiment, this invention reduces violent outbursts in a patient. In another embodiment, this invention regulates food intake. In one embodiment, food consumption is reduced. In one embodiment, food cravings are reduced. In a preferred embodiment, the patient is not addicted to cocaine or an opiate.

In some embodiments, the therapeutic dose of noribogaine or pharmaceutically acceptable salt and/or solvate thereof administered to the patient is sufficient to provide a serum concentration of about 1000 to about 6000 ng·hour/mL (area under the curve for 24 hours, AUC/24 h). In some embodiments the therapeutic dose of noribogaine, noribogaine derivative, or pharmaceutically acceptable salt or solvate thereof administered to the patient is sufficient to provide a maximum serum concentration (Cmax) of less than about 250 ng/mL. In a preferred embodiment, the therapeutic dose provides a Cmax of about 100 ng/mL to about 200 ng/mL.

In some embodiments, the therapeutic dose of noribogaine, noribogaine derivative, or pharmaceutically acceptable salt or solvate thereof administered to the patient is sufficient to provide an average serum concentration of about 50 ng/mL to about 180 ng/mL, or any subrange or subvalue there between. In a preferred embodiment, the dose of noribogaine or pharmaceutically acceptable salt and/or solvate thereof administered to the patient provides an average serum concentration of about 50 ng/mL to about 110 ng/mL. In one embodiment, the dose of noribogaine or pharmaceutically acceptable salt and/or solvate thereof administered to the patient provides an average serum concentration of about 50 ng/mL to about 100 ng/mL. In one embodiment, the dose of noribogaine or pharmaceutically acceptable salt and/or solvate thereof administered to the patient provides an average serum concentration of less than about 50 ng/mL.

In a preferred embodiment, the narrow therapeutic doses of noribogaine, noribogaine derivative, or pharmaceutically acceptable salt and/or solvate thereof described above unexpectedly do not prolong the QT interval to unacceptable levels in human patients. In some embodiments, the patient will be pre-screened to evaluate tolerance for prolongation of QT interval, e.g., to determine whether the patient has any pre-existing cardiac conditions which would disqualify him/her from treatment with noribogaine or noribogaine derivative.

In some embodiments, the serum concentration is sufficient to inhibit or ameliorate symptoms of anxiety disorders, impulse control disorder, anger/violence-related disorders, or to regulate food intake while maintaining a QT interval of less than about 500 milliseconds (ms) during said treatment. In some embodiments, the dose of noribogaine or pharmaceutically acceptable salt and/or solvate thereof maintains a QT interval of less than about 450 ms. In some embodiments, the dose of noribogaine or pharmaceutically acceptable salt and/or solvate thereof maintains a QT interval of less than about 420 ms.

In some embodiments, the dose of noribogaine or pharmaceutically acceptable salt and/or solvate thereof provides prolongation of the QT interval of less than about 50 ms. In some embodiments, the dose of noribogaine or pharmaceutically acceptable salt and/or solvate thereof provides prolongation of the QT interval of less than about 30 ms. In a preferred embodiment, the dose of noribogaine or pharmaceutically acceptable salt and/or solvate thereof provides prolongation of the QT interval of less than about 20 ms. In a preferred embodiment, the patient is tested to determine QT interval before treatment with noribogaine, and if clinician determines that the QT prolongation would be an unacceptable risk, noribogaine therapy will be contraindicated.

In another aspect, this invention provides a method for treating anxiety disorders, impulse control disorder, anger/violence-related disorders, or regulating food intake in a patient in need thereof comprising administering to the patient noribogaine or a noribogaine derivative in a sustained release manner such that the concentration of noribogaine, noribogaine derivative, pharmaceutically acceptable salt and/or solvate thereof is maintained at a therapeutically effective amount for period of about 6 hours, about 12 hours, about 18 hours, about 24 hours, about 36 hours, about 48 hours, about 72 hours, about 96 hours, or a period of time between any two of these durations.

In one aspect, provided herein is a method for treating an anxiety-related disorder in a patient in need thereof, comprising administering to the patient a therapeutically effective amount of noribogaine, noribogaine derivative, or a pharmaceutically acceptable salt and/or solvate thereof, wherein the patient is not addicted to cocaine or an opiate, and further wherein the therapeutically effective amount provides an efficacious average noribogaine serum level of between about 50 ng/mL and about 180 ng/mL while maintaining a QT interval of less than about 500 ms during said treatment.

In one aspect, provided herein is a method for treating an impulse control disorder in a patient in need thereof, comprising administering to the patient a therapeutically effective amount of noribogaine, noribogaine derivative, or a pharmaceutically acceptable salt and/or solvate thereof, wherein the patient is not addicted to cocaine or an opiate, and further wherein the therapeutically effective amount provides an efficacious average noribogaine serum level of between about 50 ng/mL and about 180 ng/mL while maintaining a QT interval of less than about 500 ms during said treatment.

In one aspect, provided herein is a method for regulating food intake and/or attenuating food craving in a patient in need thereof, comprising administering to the patient a therapeutically effective amount of noribogaine, noribogaine derivative, or a pharmaceutically acceptable salt and/or solvate thereof, wherein the patient is not addicted to cocaine or an opiate, and further wherein the therapeutically effective amount provides an efficacious average noribogaine serum level of between about 50 ng/mL and about 180 ng/mL while maintaining a QT interval of less than about 500 ms during said treatment.

In one aspect, provided herein is a method for treating an anger-related disorder in a patient in need thereof, comprising administering to the patient a therapeutically effective amount of noribogaine, noribogaine derivative, or a pharmaceutically acceptable salt and/or solvate thereof, and further wherein the therapeutically effective amount provides an efficacious average noribogaine serum level of between about 50 ng/mL and about 180 ng/mL while maintaining a QT interval of less than about 500 ms during said treatment.

In some embodiments, the maintenance dose of noribogaine is 5 mg to 100 mg. In some embodiments, the maintenance dose of noribogaine is about 1.5 mg/kg body weight. In some embodiments, the maintenance dose of noribogaine is about 1 mg/kg body weight. In some embodiments, the maintenance dose of noribogaine is about 0.9 mg/kg body weight. In some embodiments, the maintenance dose of noribogaine is about 0.8 mg/kg body weight. In some embodiments, the maintenance dose of noribogaine is about 0.7 mg/kg body weight. In some embodiments, the maintenance dose of noribogaine is about 0.6 mg/kg body weight. In some embodiments, the maintenance dose of noribogaine is about 0.5 mg/kg body weight. In some embodiments, the maintenance dose of noribogaine is about 0.4 mg/kg body weight. In some embodiments, the maintenance dose of noribogaine is about 0.3 mg/kg body weight. In some embodiments, the maintenance dose of noribogaine is about 0.2 mg/kg body weight. In some embodiments, the maintenance dose of noribogaine is about 0.1 mg/kg body weight.

### BRIEF DESCRIPTION OF THE FIGURES

FIG. 1A represents effects of noribogaine on food consumption on food maintained responding rats. Data represent mean+s.e.m. *P<0.05; ***P<0.001 compared to vehicle treatment.

FIG. 1B represents effects of noribogaine on inactive lever response in food maintained responding rats. Data represent mean+s.e.m. ***P<0.001 compared to vehicle treatment.

FIG. 2 represents mean noribogaine concentration-time profiles of healthy patients after single oral dosing with 3, 10, 30 or 60 mg doses. Inset: Individual concentration-time profiles from 0-12 h after a 10 mg dose.

FIG. 3 represents mean plasma noribogaine glucuronide concentration-time profiles after single oral 30 or 60 mg doses.

FIG. 4 illustrates the mean noribogaine concentration-time profile in opioid-addicted patients after a single oral 60 mg (diamonds), 120 mg (squares), or 180 mg (triangles) dose of noribogaine.

FIG. 5 illustrates hours to resumption of opioid substitution treatment (OST) for each patient given placebo (circles), or a single oral dose of noribogaine (60 mg, squares; 120 mg, triangles; 180 mg, inverted triangles). Center horizontal line represents mean. Error bars represent standard deviation.

FIG. 6 illustrates results of noribogaine treatment on final COWS scores before resumption of OST. Boxes include values representing 25%-75% quartiles. Diamonds represent the median, crossbars represent mean. Whiskers represent values within one standard deviation of mid-quartiles. No outliers were present.

FIG. 7A illustrates of the mean change in total COWS scores over the first 6 hours following dosing of noribogaine (60 mg, squares; 120 mg, triangles; 180 mg, diamonds) or placebo (circles). Data is given relative to baseline COWS score.

FIG. 7B illustrates the mean area under the curve (AUC) over the initial 6 hour period after administration of noribogaine or placebo, based on the COWS score data given in FIG. 7A. A negative change in score indicates that withdrawal symptoms subsided over the period.

FIG. 8A illustrates of the mean change in total OOWS scores over the first 6 hours following dosing of noribogaine (60 mg, squares; 120 mg, triangles; 180 mg, diamonds) or placebo (circles). Data is given relative to baseline OOWS score.

FIG. 8B illustrates the mean area under the curve (AUC) over the initial 6 hour period after administration of noribogaine or placebo, based on the OOWS score data given in FIG. 8A. A negative change in score indicates that withdrawal symptoms subsided over the period.

FIG. 9A illustrates of the mean change in total SOWS scores over the first 6 hours following dosing of noribogaine (60 mg, squares; 120 mg, triangles; 180 mg, diamonds) or placebo (circles). Data is given relative to baseline SOWS score.

FIG. 9B illustrates the mean area under the curve (AUC) over the initial 6 hour period after administration of noribogaine or placebo, based on the SOWS score data given in FIG. 9A. A negative change in score indicates that withdrawal symptoms subsided over the period.

FIG. 10A illustrates the average change in QT interval (ΔQTcl) for each cohort (60 mg, squares; 120 mg, triangles; 180 mg, diamonds) or placebo (circles) over the first 24 hours post administration.

FIG. 10B illustrates the correlation between serum noribogaine concentration and ΔQTcl for each patient over time. The equation of the line is given.

FIG. 11 shows the effects of noribogaine treatment (at 1-, 5- and 10-mg/L doses) on general motor activity of zebrafish. Behavioral endpoints examined include: latency to upper half of tank (panel A), transitions to upper half of tank (panel B), transitions to upper half of tank per minute (panel C), time in upper half of tank (panel D), time in upper half of tank per minute (panel E), average entry duration (panel F), and average entry duration per minute (panel G).

FIG. 12 shows the effects of noribogaine treatment (at 1-, 5- and 10-mg/L doses) on general motor activity of zebrafish. Behavioral endpoints examined include: distance moved (panel A), velocity (panel B), rotation angle (panel C), number of rotation events (panel D), change in direction of body/heading (panel E), change in direction of movement per distance moved/meander total (panels F and G).

FIG. 13 depicts effects of noribogaine (at 1-, 5- and 10-mg/L doses) on freezing bouts frequency (panel A) and duration of freezing bouts (panel B).

FIG. 14 depicts effects of noribogaine treatment on movement mobility, including percentage of events per animal (panel A) and duration (panel B). “Immobile” (high frequency “HF” or low frequency “LF”) was used to express the frequency of episodes with degree of movement independent of spatial displacement (duration of immobility). Mobile (HF or LF) reflects frequency of episodes of moderate locomotor activity. Hi-mobile (HF and LF) reflects bouts of accelerated velocity (>60% of individual average).

FIG. 15 shows representative traces of control (top row) and noribogaine-treated fish (1, 5 and 10 mg/L, from top to bottom), recorded in the 5 minute novel tank test (NTT) by Ethovision XT8.5 software.

### DETAILED DESCRIPTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds.

### DEFINITIONS

Unless defined otherwise, all technical and scientific terms used herein have the same meaning as commonly understood by one of ordinary skill in the art to which this invention belongs. As used herein the following terms have the following meanings.

The term “about” when used before a numerical designation, e.g., temperature, time, amount, concentration, and such other, including a range, indicates approximations which may vary by (+) or (−) 20%, 10%, 5%, 1%, or any subrange or subvalue there between. Preferably, the term “about” when used with regard to a dose amount means that the dose may vary by +/−20%. For example, “about 2 mg/kg noribogaine” indicates that a patient may be administered a dose of noribogaine between 1.6 mg/kg and 2.4 mg/kg. In another example, about 120 mg per unit dose of noribogaine indicates that the unit dose may range from 96 mg to 144 mg.

“Administration” refers to introducing an agent, such as noribogaine, into a patient. Typically, an effective amount is administered, which amount can be determined by the treating physician or the like. Any route of administration, such as oral, topical, subcutaneous, peritoneal, intra-arterial, inhalation, vaginal, rectal, nasal, introduction into the cerebrospinal fluid, or instillation into body compartments can be used. The agent, such as noribogaine may be administered by direct blood stream delivery, e.g. sublingual, buccal, intranasal, or intrapulmonary administration. The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Periodic administration” or “periodically administering” refers to multiple treatments that occur on a daily, weekly, or monthly basis. Periodic administration may also refer to administration of an agent, such as noribogaine one, two, three, or more times per day. Administration may be via transdermal patch, gum, lozenge, sublingual tablet, intranasal, intrapulmonary, oral administration, or other administration.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the term “alkyl” refers to monovalent saturated aliphatic hydrocarbyl groups having from 1 to 12 carbon atoms, 1 to 10 carbon atoms, preferably 1 to 6 carbon atoms, and more preferably 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term “Cₓ alkyl” refers to an alkyl group having x carbon atoms, wherein x is an integer, for example, C₃ refers to an alkyl group having 3 carbon atoms.

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR³⁸C(O)alkyl, —NR³⁸C(O) substituted alkyl, —NR³⁸C(O)cycloalkyl, —NR³⁸C(O) substituted cycloalkyl, —NR³⁸C(O)cycloalkenyl, —NR³⁸C(O) substituted cycloalkenyl, —NR³⁸C(O)alkenyl, —NR³⁸C(O) substituted alkenyl, —NR³⁸C(O)alkynyl, —NR³⁸C(O) substituted alkynyl, —NR³⁸C(O)aryl, —NR³⁸C(O) substituted aryl, —NR³⁸C(O)heteroaryl, —NR³⁸C(O) substituted heteroaryl, —NR³⁸C(O)heterocyclic, and —NR³⁸C(O) substituted heterocyclic wherein R³⁸ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³⁹R⁴⁰ where R³⁹ and R⁴⁰ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R³⁹ and R⁴⁰ are optionally joined, together with the nitrogen bound thereto to form a heterocyclic or substituted heterocyclic group, provided that R³⁹ and R⁴⁰ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R³⁹ is hydrogen and R⁴⁰ is alkyl, the substituted amino group is sometimes referred to herein as alkylamino. When R³⁹ and R⁴⁰ are alkyl, the substituted amino group is sometimes referred to herein as dialkylamino. When referring to a monosubstituted amino, it is meant that either R³⁹ or R⁴⁰ is hydrogen but not both. When referring to a disubstituted amino, it is meant that neither R³⁹ nor R⁴⁰ are hydrogen.

“Aminocarbonyl” refers to the group —C(O)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR³⁸C(O)NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR³⁸C(S)NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁴¹R⁴² where R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR³⁸—SO₂NR⁴¹R⁴² where R³⁸ is hydrogen or alkyl and R⁴¹ and R⁴² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁴³)NR⁴¹R⁴² where R⁴¹, R⁴², and R⁴³ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁴¹ and R⁴² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR³⁸—C(O)O-alkyl, —NR³⁸—C(O)O-substituted alkyl, —NR³⁸—C(O)O-alkenyl, —NR³⁸—C(O)O-substituted alkenyl, —NR³⁸—C(O)O-alkynyl, —NR^{3′}—C(O)O-substituted alkynyl, —NR³⁸—C(O)O-aryl, —NR³⁸—C(O)O-substituted aryl, —NR³⁸—C(O)O-cycloalkyl, —NR³⁸—C(O)O-substituted cycloalkyl, —NR³⁸—C(O)O-cycloalkenyl, —NR³⁸—C(O)O-substituted cycloalkenyl, —NR³⁸—C(O)O-heteroaryl, —NR³⁸—C(O)O-substituted heteroaryl, —NR³⁸—C(O)O-heterocyclic, and —NR³⁸—C(O)O-substituted heterocyclic wherein R³⁸ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, substituted —O—C(O)O-alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2]octanyl, norbornyl, and spirobicyclo groups such as spiro[4.5]dec-8-yl.

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁴⁴C(═NR⁴⁴)N(R⁴⁴)₂ where each R⁴⁴ is independently selected from the group consisting of hydrogen, alkyl, substituted alkyl, aryl, substituted aryl, heteroaryl, substituted heteroaryl, heterocyclic, and substituted heterocyclic and two R⁴⁴ groups attached to a common guanidino nitrogen atom are optionally joined together with the nitrogen bound thereto to form a heterocyclic or substituted heterocyclic group, provided that at least one R⁴⁴ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridyl, pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, and/or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic heterocyclic ring. In one embodiment, the nitrogen and/or sulfur atom(s) of the heterocyclic group are optionally oxidized to provide for the N-oxide, sulfinyl, and/or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl), and the term “(substituted sulfonyl)aminocarbonyl” refers to —C(O)NH(substituted sulfonyl), wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Compound” or “compounds” as used herein is meant to include the stereoisomers and tautomers of the indicated formulas.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

As used herein, the term “phosphate ester” refers to any one of the mono-, di- or triphosphate esters of noribogaine, wherein the mono-, di- or triphosphate ester moiety is bonded to the 12-hydroxy group and/or the indole nitrogen of noribogaine.

As used herein, the term “phosphate ester” refers to any one of the mono-, di- or triphosphate esters of noribogaine, wherein the mono-, di- or triphosphate ester moiety is bonded to the 12-hydroxy group and/or the indole nitrogen of noribogaine.

As used herein, the term “monophosphate” refers to the group —P(O)(OH)₂.

As used herein, the term “diphosphate” refers to the group —P(O)(OH)—OP(O)(OH)₂.

As used herein, the term “triphosphate” refers to the group —P(O)(OH)—(OP(O)(OH))₂OH.

As used herein, the term “ester” as it refers to esters of the mono-, di- or triphosphate group means esters of the monophosphate can be represented by the formula —P(O)(OR⁴⁵)₂, where each R⁴⁵ is independently hydrogen, C₁-C₁₂ alkyl, C₃-C₁₀ cycloalkyl, C₆-C₁₄ aryl, heteroaryl of 1 to 10 carbon atoms and 1 to 4 optionally oxidized heteroatoms selected from the group consisting of oxygen, nitrogen, and sulfur and the like, provided that at least one R⁴⁵ is not hydrogen. Likewise, exemplary esters of the di- or triphosphate can be represented by the formulas —P(O)(OR⁴⁵)—OP(O)(OR⁴⁵)₂ and —P(O)(OR⁴⁵)—(OP(O)(OR⁴⁵))₂OR⁴⁵, where R⁴⁵ is as defined above.

As used herein, the term “hydrolyzable group” refers to a group that can be hydrolyzed to release the free hydroxy group under hydrolysis conditions. Examples of hydrolysable group include, but are not limited to those defined for R above. Preferred hydrolysable groups include carboxyl esters, phosphates and phosphate esters. The hydrolysis may be done by chemical reactions conditions such as base hydrolysis or acid hydrolysis or may be done in vivo by biological processes, such as those catalyzed by a phosphate hydrolysis enzyme. Nonlimiting examples of hydrolysable group include groups linked with an ester-based linker (—C(O)O— or —OC(O)—), an amide-based linker (—C(O)NR⁴⁶— or —NR⁴⁶C(O)—), or a phosphate-linker (—P(O)(OR⁴⁶)—O—, —O—P(S)(OR⁴⁶)—O—, —O—P(S)(SR⁴⁶)—O—, —S—P(O)(OR⁴⁶)—O—, —O—P(O)(OR⁴⁶)—S—, —S—P(O)(OR⁴⁶)—S—, —O—P(S)(OR⁴⁶)—S—, —S—P(S)(OR⁴⁶)—O—, —O—P(O)(R⁴⁶)—O—, —O—P(S)(R⁴⁶)—O—, —S—P(O)(R⁴⁶)—O—, —S—P(S)(R⁴⁶)—O—, —S—P(O)(R⁴⁶)—S—, or —O—P(S)(R⁴⁶)—S—) where R⁴⁶ can be hydrogen or alkyl.

Substituted groups of this invention, as set forth above, do not include polymers obtained by an infinite chain of substituted groups. At most, any substituted group can be substituted up to five times.

“Noribogaine” refers to the compound:

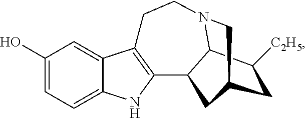

as well as noribogaine derivatives, or pharmaceutically acceptable salts and pharmaceutically acceptable solvates thereof. It should be understood that where “noribogaine” is mentioned herein, one more polymorphs of noribogaine can be utilized and are contemplated. In some embodiments, noribogaine is noribogaine glucuronide. Noribogaine can be prepared by demethylation of naturally occurring ibogaine:

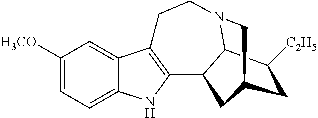

which is isolated from Tabernanth iboga, a shrub of West Africa. Demethylation may be accomplished by conventional techniques such as by reaction with boron tribromide/methylene chloride at room temperature followed by conventional purification. See, for example, Huffman, et al., J. Org. Chem. 50:1460 (1985), which is incorporated herein by reference in its entirety. Noribogaine can be synthesized as described, for example in U.S. Patent Pub. Nos. 2013/0165647, 2013/0303756, and 2012/0253037, PCT Patent Publication No. WO 2013/040471 (includes description of making noribogaine polymorphs), and U.S. patent application Ser. No. 13/593,454, each of which is incorporated herein by reference in its entirety.

“Noribogaine derivatives” refer to, without limitation, esters or O-carbamates of noribogaine, or pharmaceutically acceptable salts and/or solvates of each thereof. Also encompassed within this invention are derivatives of noribogaine that act as prodrug forms of noribogaine. A prodrug is a pharmacological substance administered in an inactive (or significantly less active) form. Once administered, the prodrug is metabolized in vivo into an active metabolite. Noribogaine derivatives include, without limitation, those compounds set forth in U.S. Pat. Nos. 6,348,456 and 8,362,007; as well as in U.S. patent application Ser. No. 13/165,626; and US Patent Application Publication Nos. US2013/0131046; US2013/0165647; US2013/0165425; and US2013/0165414; all of which are incorporated herein by reference. Non-limiting examples of noribogaine derivatives encompassed by this invention are given in more detail in the “Compositions” section below.

In some embodiments, the methods of the present disclosure entail the administration of a prodrug of noribogaine that provides the desired maximum serum concentrations and efficacious average noribogaine serum levels. A prodrug of noribogaine refers to a compound that metabolizes, in vivo, to noribogaine. In some embodiments, the prodrug is selected to be readily cleavable either by a cleavable linking arm or by cleavage of the prodrug entity that binds to noribogaine such that noribogaine is generated in vivo. In one preferred embodiment, the prodrug moiety is selected to facilitate binding to the g and/or K receptors in the brain either by facilitating passage across the blood brain barrier or by targeting brain receptors other than the g and/or K receptors. Examples of prodrugs of noribogaine are provided in U.S. patent application Ser. No. 13/165,626, the entire content of which is incorporated herein by reference.

This invention is not limited to any particular chemical form of noribogaine or noribogaine derivative, and the drug may be given to patients either as a free base, solvate, or as a pharmaceutically acceptable acid addition salt. In the latter case, the hydrochloride salt is generally preferred, but other salts derived from organic or inorganic acids may also be used. Examples of such acids include, without limitation, those described below as “pharmaceutically acceptable salts” and the like. As discussed above, noribogaine itself may be formed from the 0-demethylation of ibogaine which, in turn, may be synthesized by methods known in the art (see e.g., Huffman, et al., J. Org. Chem. 50:1460 (1985)).

“Pharmaceutically acceptable composition” refers to a composition that is suitable for administration to a mammal, preferably a human. Such compositions include various excipients, diluents, carriers, and such other inactive agents well known to the skilled artisan.

“Pharmaceutically acceptable salt” refers to pharmaceutically acceptable salts, including pharmaceutically acceptable partial salts, of a compound, which salts are derived from a variety of organic and inorganic counter ions well known in the art and include, by way of example only, hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, oxalic acid and the like, and when the molecule contains an acidic functionality, include, by way of example only, sodium, potassium, calcium, magnesium, ammonium, tetraalkylammonium, and the like.

“Therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, in the context of treating anxiety disorders, impulse control disorder, and/or anger/violence-related disorders, refers to an amount of the agent that attenuates the anxiety disorder, impulse control disorder, or anger/violence-related disorders, and/or symptoms thereof, in the patient. A therapeutically effective amount of an agent, in the context of regulating food intake and/or controlling food cravings, refers to an amount of the agent that reduces the patient's food intake and/or reduces food cravings in the patient.

The therapeutically effective amount of the compound may be higher or lower, depending on the route of administration used. For example, when direct blood administration (e.g., sublingual, pulmonary and intranasal delivery) is used, a lower dose of the compound may be administered. In one aspect, a therapeutically effective amount of noribogaine or derivative is from about 50 ng to less than 100 μg per kg of body weight. Where other routes of administration may be used, a higher dose of the compound is administered. In one embodiment, the therapeutically effective amount of the compound is from about 1 mg to about 4 mg per kg of body weight per day.

A “therapeutic level” of a drug is an amount of noribogaine, noribogaine derivative, or pharmaceutical salt or solvate thereof that is sufficient to treat a disease or disorder or symptoms of a disease or disorder or to treat, prevent, or attenuate a disease or disorder or symptoms of a disease or disorder but not high enough to pose any significant risk to the patient. Therapeutic levels of drugs can be determined by tests that measure the actual concentration of the compound in the blood of the patient. This concentration is referred to as the “serum concentration.” Where the serum concentration of noribogaine is mentioned, it is to be understood that the term “noribogaine” encompasses any form of noribogaine, including derivatives thereof.

A “sub-therapeutic level” of noribogaine or pharmaceutical salt and/or solvate thereof that is less than the therapeutic level described above. For example, the sub-therapeutic level of noribogaine may be e.g., 80%, 70%, 60%, 50%, 40%, 30%, 20%, or 10% less than a therapeutically effective amount (e.g., 120 mg) of noribogaine, or any subvalue or subrange there between. Sub-therapeutic levels of noribogaine may coincide with “maintenance amounts” of noribogaine which are amounts, less than the therapeutically effective amount, that provide some attenuation and/or prevention of post-acute withdrawal syndrome in a patient. The maintenance amount of the compound is expected to be less than the therapeutically effective amount because the level of inhibition does not need to be as high in a patient who is no longer physically addicted to opioid or opioid-like drug.

The term “dose” refers to a range of noribogaine, noribogaine derivative, or pharmaceutical salt or solvate thereof that provides a therapeutic serum level of noribogaine when given to a patient in need thereof. The dose is recited in a range, for example from about 20 mg to about 120 mg, and can be expressed either as milligrams or as mg/kg body weight. The attending clinician will select an appropriate dose from the range based on the patient's weight, age, degree of addiction, health, and other relevant factors, all of which are well within the skill of the art.

The term “unit dose” refers to a dose of drug that is given to the patient to provide therapeutic results, independent of the weight of the patient. In such an instance, the unit dose is sold in a standard form (e.g., 20 mg tablet). The unit dose may be administered as a single dose or a series of subdoses. In some embodiments, the unit dose provides a standardized level of drug to the patient, independent of weight of patient. Many medications are sold based on a dose that is therapeutic to all patients based on a therapeutic window. In such cases, it is not necessary to titrate the dosage amount based on the weight of the patient.

As defined herein, a “prophylactically effective amount” of a drug is an amount, typically less than the therapeutically effective amount, that provides attenuation and/or prevention of a disease or disorder or symptoms of a disease or disorder in a patient. For example, the prophylactically effective amount of the compound is expected to be less than the therapeutically effective amount because the level of inhibition does not need to be as high in a patient who no longer has a disease or disorder or symptoms of a disease or disorder (e.g., no longer physically addicted to nicotine). For example, a prophylactically effective amount is preferably 90%, 80%, 70%, 60%, 50%, 40%, 30%, 20%, or 10% less than a therapeutically effective amount. However, a prophylactically effective amount may be the same as the therapeutically effective amount, for example when a patient who is physically addicted to nicotine is administered noribogaine to attenuate cravings for a period of time when nicotine use is not feasible. The prophylactically effective amount may vary for different a diseases or disorders or symptoms of different diseases or disorders.

As defined herein, a “maintenance amount” of a drug or an agent is an amount, typically less than the therapeutically effective amount that provides attenuation and/or prevention of syndrome disease or disorder or symptoms of a disease or disorder in a patient. The maintenance amount of the compound is expected to be less than the therapeutically effective amount because the level of inhibition does not need to be as high in a patient who is no longer physically manifests a disease or disorder or symptoms of a disease or disorder. For example, a maintenance amount is preferably 90%, 80%, 70%, 60%, 50%, 40%, 30%, 20%, or 10% less than a therapeutically effective amount, or any subvalue or subrange there between.

“Treatment”, “treating”, and “treat” are defined as acting upon a disease, disorder, or condition with an agent to reduce or ameliorate harmful or any other undesired effects of the disease, disorder, or condition and/or its symptoms. “Treatment,” as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition. “Treating” or “treatment of” a condition or patient refers to taking steps to obtain beneficial or desired results, including clinical results such as the reduction of symptoms. For purposes of this invention, beneficial or desired clinical results include, but are not limited to, treating an anxiety disorder, impulse control disorder, anger/violence-related disorders, or regulating food intake. Anxiety disorders or impulse control disorders occurring as a result of withdraw and/or use of an opiate or other illicit drug or substance is not within the scope of this invention.

As used herein, the term “QT interval” refers to the measure of the time between the start of the Q wave and the end of the T wave in the electrical cycle of the heart. Prolongation of the QT interval refers to an increase in the QT interval.

As used herein, the term “patient” refers to mammals and includes humans and non-human mammals.

A “pharmaceutically acceptable solvate” or “hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

As used herein, the term “solvate” is taken to mean that a solid-form of a compound that crystallizes with one or more molecules of solvent trapped inside. A few examples of solvents that can be used to create solvates, such as pharmaceutically acceptable solvates, include, but are certainly not limited to, water, methanol, ethanol, isopropanol, butanol, C1-C6 alcohols in general (and optionally substituted), tetrahydrofuran, acetone, ethylene glycol, propylene glycol, acetic acid, formic acid, water, and solvent mixtures thereof. Other such biocompatible solvents which may aid in making a pharmaceutically acceptable solvate are well known in the art and applicable to the present invention. Additionally, various organic and inorganic acids and bases can be added or even used alone as the solvent to create a desired solvate. Such acids and bases are known in the art. When the solvent is water, the solvate can be referred to as a hydrate. Further, by being left in the atmosphere or recrystallized, the compounds of the present invention may absorb moisture, may include one or more molecules of water in the formed crystal, and thus become a hydrate. Even when such hydrates are formed, they are included in the term “solvate”. Solvate also is meant to include such compositions where another compound or complex co-crystallizes with the compound of interest. The term “solvate” as used herein refers to complexes with solvents in which noribogaine is reacted or from which noribogaine is precipitated or crystallized. For example, a complex with water is known as a “hydrate”. Solvates of noribogaine are within the scope of the invention. It will be appreciated by those skilled in organic chemistry that many organic compounds can exist in more than one crystalline form. For example, crystalline form may vary based on the solvate used. Thus, all crystalline forms of noribogaine or the pharmaceutically acceptable solvates thereof are within the scope of the present invention.

### Compositions

As will be apparent to the skilled artisan upon reading this disclosure, this invention provides compositions for treating an anxiety disorder, impulse control disorder, anger/violence-related disorders, or regulating food intake, in a subject, comprising noribogaine, noribogaine derivatives, prodrugs of noribogaine, pharmaceutically acceptable salts and/or solvates of each thereof. This invention further provides compositions for treating, attenuating, or preventing anxiety disorder, impulse control disorder, anger/violence-related disorders, symptoms thereof, or food cravings in a subject, comprising noribogaine, noribogaine derivatives, prodrugs of noribogaine, pharmaceutically acceptable salts and/or solvates of each thereof.

In some embodiments, the composition is formulated for sublingual, intranasal, or intrapulmonary delivery. In one aspect, the invention provides a pharmaceutical composition comprising a pharmaceutically effective amount of noribogaine and a pharmaceutically acceptable excipient, wherein the therapeutically effective amount of noribogaine is an amount that delivers an aggregate amount of noribogaine of about 50 ng to less than about 100 μg per kg body weight per day. In some aspects, the therapeutically effective amount of noribogaine is an amount that delivers an aggregate amount of noribogaine of about 50 ng to about 50 μg per kg body weight per day. In some aspects, the therapeutically effective amount of noribogaine is an amount that delivers an aggregate amount of noribogaine of about 50 ng to about 10 μg per kg body weight per day. In some aspects, the therapeutically effective amount of noribogaine is an amount that delivers an aggregate amount of noribogaine of about 50 ng to about 1 μg per kg body weight per day. In some aspects, the composition is formulated for administration once per day. In some aspects, the composition is formulated for administration two or more times per day. The ranges include both extremes as well as any subranges there between.

In some embodiments, the composition is formulated for oral, buccal, transdermal, internal, pulmonary, rectal, nasal, vaginal, lingual, intravenous, intraarterial, intramuscular, intraperitoneal, intracutaneous or subcutaneous delivery.

In one embodiment, the therapeutically effective amount of the compound is from about 1 mg to about 4 mg per kg body weight per day. In another embodiment, the therapeutically effective amount of the compound is from about 1 mg to about 3 mg per kg body weight per day. In another embodiment, the therapeutically effective amount of the compound is from about 1 mg to about 2 mg per kg body weight per day. In another embodiment, the therapeutically effective amount of the compound is from about 1.3 mg to about 3 mg per kg body weight per day. In another embodiment, the therapeutically effective amount of the compound is from about 1.5 mg to about 3 mg per kg body weight per day. In another embodiment, the therapeutically effective amount of the compound is from about 1.7 mg to about 3 mg per kg body weight per day. In another embodiment, the therapeutically effective amount of the compound is from about 1.3 mg to about 4 mg per kg body weight per day. In another embodiment, the therapeutically effective amount of the compound is from about 1.5 mg to about 4 mg per kg body weight per day. In another embodiment, the therapeutically effective amount of the compound is about about 2 mg per kg body weight per day. The ranges include both extremes as well as any subranges there between.

In one embodiment, the therapeutically effective amount of the compound is about 4 mg/kg body weight per day. In one embodiment, the therapeutically effective amount of the compound is about 3 mg/kg body weight per day. In another embodiment, the therapeutically effective amount of the compound is about 2 mg per kg body weight per day. In another embodiment, the therapeutically effective amount of the compound is about 1.7 mg per kg body weight per day. In another embodiment, the therapeutically effective amount of the compound is about 1.5 mg per kg body weight per day. In another embodiment, the therapeutically effective amount of the compound is about 1.2 mg per kg body weight per day. In another embodiment, the therapeutically effective amount of the compound is about 1 mg per kg body weight per day.

### Compounds Utilized

In one embodiment, the noribogaine derivative is represented by Formula I:

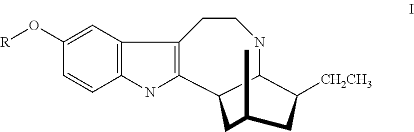

or a pharmaceutically acceptable salt and/or solvate thereof,
wherein R is hydrogen or a hydrolyzable group such as hydrolyzable esters of from about 1 to 12 carbons.

Generally, in the above formula, R is hydrogen or a group of the formula:

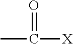

wherein X is a C₁-C₁₂ group, which is unsubstituted or substituted. For example, X may be a linear alkyl group such as methyl, ethyl, n-propyl, n-butyl, n-pentyl, n-hexyl, n-heptyl, n-octyl, n-nonyl, n-decyl, n-undecyl or n-dodecyl, or a branched alkyl group, such as i-propyl or sec-butyl. Also, X may be a phenyl group or benzyl group, either of which may be substituted with lower alkyl groups or lower alkoxy groups. Generally, the lower alkyl and/or alkoxy groups have from 1 to about 6 carbons. For example, the group R may be acetyl, propionyl or benzoyl. However, these groups are only exemplary.

Generally, for all groups X, they may either be unsubstituted or substituted with lower alkyl or lower alkoxy groups. For example, substituted X may be o-, m- or p-methyl or methoxy benzyl groups.

C₁-C₁₂ groups include C₁-C₁₂ alkyl, C₃-C₁₂ cycloalkyl, C₆-C₁₂ aryl, C₇-C₁₂ arylalkyl, wherein Cₓ indicates that the group contains x carbon atoms. Lower alkyl refers to C₁-C₄ alkyl and lower alkoxy refers to C₁-C₄ alkoxy.

In one embodiment, the noribogaine derivative is represented by Formula II:

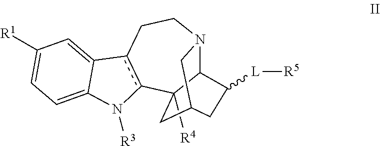

or a pharmaceutically acceptable salt and/or solvate thereof,
wherein
[symbol] is a single or double bond;
R¹ is halo, OR², or C₁-C₁₂ alkyl optionally substituted with 1 to 5 R¹⁰;
R² is hydrogen or a hydrolysable group selected from the group consisting of —C(O)R^{x}, —C(O)OR^{x} and —C(O)N(R^{y})₂ where each R^{x} is selected from the group consisting of C₁-C₆ alkyl optionally substituted with 1 to 5 R¹⁰, and each R^{y} is independently selected from the group consisting of hydrogen, C₁-C₆ alkyl optionally substituted with 1 to 5 R¹⁰, C₆-C₁₄ aryl optionally substituted with 1 to 5 R¹⁰, C₃-C₁₀ cycloalkyl optionally substituted with 1 to 5 R¹⁰, C₁-C₁₀ heteroaryl having 1 to 4 heteroatoms and which is optionally substituted with 1 to 5 R¹⁰, C₁-C₁₀ heterocyclic having 1 to 4 heteroatoms and which is optionally substituted with 1 to 5 R¹⁰, and where each R^{y}, together with the nitrogen atom bound thereto form a C₁-C₆ heterocyclic having 1 to 4 heteroatoms and which is optionally substituted with 1 to 5 R¹⁰ or a C₁-C₆ heteroaryl having 1 to 4 heteroatoms and which is optionally substituted with 1 to 5 R¹⁰;
R³ is selected from the group consisting of hydrogen, C₁-C₁₂ alkyl optionally substituted with 1 to 5 R¹⁰, aryl optionally substituted with 1 to 5 R¹⁰, —C(O)R⁶, —C(O)NR⁶R⁶ and —C(O)OR⁶;
R⁴ is selected from the group consisting of hydrogen, —(CH₂)ₘOR⁸, —CR⁷(OH)R⁸, —(CH₂)ₘCN, —(CH₂)ₘCOR⁸, —(CH₂)ₘCO₂R⁸, —(CH₂)ₘC(O)NR⁷R⁸, —(C H₂)ₘC(O)NR⁷NR⁸R⁸, —(CH₂)ₘC(O)NR⁷NR⁸C(O)R⁹, and —(CH₂)ₘNR⁷R⁸;
m is 0, 1, or 2;
L is a bond or C₁-C₁₂ alkylene;
R⁵ is selected from the group consisting of hydrogen, C₁-C₁₂ alkyl substituted with 1 to 5 R¹⁰, C₁-C₁₂ alkenyl substituted with 1 to 5 R¹⁰, —X¹—R⁷, —(X¹—Y)ₙ—X¹—R⁷, —SO₂NR⁷R⁸, —O—C(O)R⁹, —C(O)OR⁸, —C(O)NR⁷R⁸, —NR⁷R⁸, —NHC(O)R⁹, and —NR⁷C(O)R⁹;
each R⁶ is independently selected from the group consisting of hydrogen, C₁-C₁₂ alkyl, C₂-C₁₂ alkenyl, C₂-C₁₂ alkynyl, C₆-C₁₀ aryl, C₁-C₆ heteroaryl having 1 to 4 heteroatoms, and C₁-C₆ heterocycle having 1 to 4 heteroatoms, and wherein the alkyl, alkenyl, alkynyl, aryl, heteroaryl, and heterocycle are optionally substituted with 1 to 5 R¹⁰;

X¹ is selected from the group consisting of O and S;
Y is C₁-C₄ alkylene or C₆-C₁₀ arylene, or a combination thereof;
n is 1, 2, or 3;

R⁷ and R⁸ are each independently selected from the group consisting of hydrogen, C₁-C₁₂ alkyl optionally substituted with 1 to 5 R¹⁰, C₁-C₆ heterocycle having 1 to 4 heteroatoms and which is optionally substituted with 1 to 5 R¹⁰, C₃-C₁₀ cycloalkyl optionally substituted with 1 to 5 R¹⁰, C₆-C₁₀ aryl optionally substituted with 1 to 5 R¹⁰ and C₁-C₆ heteroaryl having 1 to 4 heteroatoms optionally substituted with 1 to 5 R¹⁰;
R⁹ is selected from the group consisting of C₁-C₁₂ alkyl optionally substituted with 1 to 5 R¹⁰, C₁-C₆ heterocycle having 1 to 4 heteroatoms optionally substituted with 1 to 5 R¹⁰, C₃-C₁₀ cycloalkyl optionally substituted with 1 to 5 R¹⁰, C₆-C₁₀ aryl optionally substituted with 1 to 5 R¹⁰ and C₁-C₆ heteroaryl having 1 to 4 heteroatoms optionally substituted with 1 to 5 R¹⁰;

R¹⁰ is selected from the group consisting of C₁-C₄ alkyl, phenyl, halo, —OR¹¹, —CN, —COR¹¹, —CO₂R¹¹, —C(O)NHR¹¹, —NR¹¹R¹¹, —C(O)NR¹¹R¹¹, —C(O)NHNHR¹¹, —C(O)NR¹¹NHR¹¹, —C(O)NR¹¹NR¹¹R¹¹, —C(O)NHNR¹¹C(O)R¹¹, —C(O)NHNHC(O) R¹¹, —SO₂NR¹¹R¹¹, —C(O)NR¹¹NR¹¹C(O)R¹¹, and —C(O)NR¹¹NHC(O)R¹¹; and
R¹¹ is independently hydrogen or C₁-C₁₂ alkyl;
provided that:
when L is a bond, then R⁵ is not hydrogen;
when [symbol] is a double bond, R¹ is an ester hydrolyzable group, R³ and R⁴ are both hydrogen, then -L-R⁵ is not ethyl;
when [symbol] is a double bond, R¹ is —OH, halo or C₁-C₁₂ alkyl optionally substituted with 1 to 5 R¹⁰, then R⁴ is hydrogen; and
when [symbol] is a double bond, R¹ is OR², R⁴ is hydrogen, -L-R⁵ is ethyl, then R² is not a hydrolyzable group selected from the group consisting of an ester, amide, carbonate and carbamate.

In one embodiment, the noribogaine derivative is represented by Formula III:

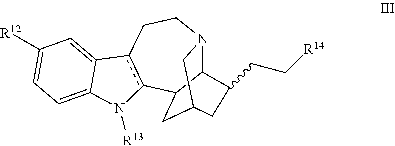

or a pharmaceutically acceptable salt and/or solvate thereof,
wherein
[symbol] is a single or double bond;
R¹² is halo, —OH, —SH, —NH₂, —S(O)₂N(R¹⁷)₂, —R^{z}-L¹-R¹⁸, —R^{z}-L¹-R¹⁹, —R^{z}-L-R²⁰ or —R^{z}-L¹-CHR¹⁸R¹⁹, where R^{z} is O, S or NR¹⁷;
L¹ is alkylene, arylene, —C(O)-alkylene, —C(O)-arylene, —C(O)O-arylene, —C(O)O— alkylene, —C(O)NR²⁰-alkylene, —C(O)NR²⁰-arylene, —C(NR²⁰)NR²⁰-alkylene or —C(NR²⁰)NR²⁰-arylene, wherein L¹ is configured such that —O-L¹-R¹⁸ is —OC(O)-alkylene-R¹⁸, —OC(O)O-arylene-R¹⁸, —OC(O)O-alkylene-R¹⁸, —OC(O)-arylene-R¹⁸, —OC(O)NR²⁰-alkylene-R¹⁸, —OC(O)NR²⁰-arylene-R¹⁸, —OC(NR²⁰)NR²⁰-alkylene-R¹⁸ or —OC(NR²⁰)NR²⁰-arylene-R¹⁸, and wherein the alkylene and arylene are optionally substituted with 1 to 2 R¹⁶;
R¹³ is hydrogen, —S(O)₂OR²⁰, —S(O)₂R²⁰, —C(O)R¹⁵, —C(O)NR¹⁵R¹⁵, —C(O)OR¹⁵, C₁-C₁₂ alkyl optionally substituted with 1 to 5 R¹⁶, C₁-C₁₂ alkenyl optionally substituted with 1 to 5 R¹⁶, or aryl optionally substituted with 1 to 5 R¹⁶;
R¹⁴ is hydrogen, halo, —OR¹⁷, —CN, C₁-C₁₂ alkyl, C₁-C₁₂ alkoxy, aryl or aryloxy, where the alkyl, alkoxy, aryl, and aryloxy are optionally substituted with 1 to 5 R¹⁶;
each R¹⁵ is independently selected from the group consisting of hydrogen, C₁-C₁₂ alkyl, C₂-C₁₂ alkenyl, C₂-C₁₂ alkynyl, aryl, heteroaryl, and heterocycle, and wherein the alkyl, alkenyl, alkynyl, aryl, heteroaryl, and heterocycle are optionally substituted with 1 to 5 R¹⁶;
R¹⁶ is selected from the group consisting of phenyl, halo, —OR¹⁷, —CN, —COR¹⁷, —CO₂R¹⁷, —NR¹⁷R¹⁷, —NR¹⁷C(O)R¹⁷, —NR¹⁷SO₂R¹⁷, —C(O)NR¹⁷R¹⁷, —C(O)NR¹⁷NR¹⁷R¹⁷, —SO₂NR¹⁷R¹⁷ and —C(O)NR¹⁷NR¹⁷C(O)R¹⁷;
each R¹⁷ is independently hydrogen or C₁-C₁₂ alkyl optionally substituted with from 1 to 3 halo;
R¹⁸ is hydrogen, —C(O)R²⁰, —C(O)OR²⁰, —C(O)N(R²⁰)₂ or —N(R²⁰)C(O)R²⁰;
R¹⁹ is hydrogen, —N(R²⁰)₂, —C(O)N(R²⁰)₂, —C(NR²⁰)N(R²⁰)₂, —C(NSO₂R²⁰)N(R²⁰)₂, —NR²⁰C(O)N(R²⁰)₂, —NR²⁰C(S)N(R²⁰)₂, —NR²⁰C(NR²⁰)N(R²⁰)₂, —NR²⁰C(NSO₂R²⁰)N(R²⁰)₂ or tetrazole; and
each R²⁰ is independently selected from the group consisting of hydrogen, C₁-C₁₂ alkyl and aryl;
provided that:
when [symbol] is a double bond and R¹³ and R¹⁴ are hydrogen, then R¹² is not hydroxy;
when [symbol] is a double bond, R¹⁴ is hydrogen, R¹² is —O-L¹-R¹⁸, —O-L¹-R¹⁹, —O-L¹-R²⁰, and L¹ is alkylene, then —O-L¹-R¹⁸, —O-L¹-R¹⁹, —O-L¹-R²⁰ are not methoxy;
when [symbol] is a double bond, R¹⁴ is hydrogen, R^{z} is O, L¹ is —C(O)-alkylene, —C(O)-arylene, —C(O)O-arylene, —C(O)O-alkylene, —C(O)NR²⁰-alkylene, or —C(O)NR²⁰-arylene, then none of R¹⁸, R¹⁹ or R²⁰ are hydrogen.

In one embodiment, the noribogaine derivative is represented by Formula IV:

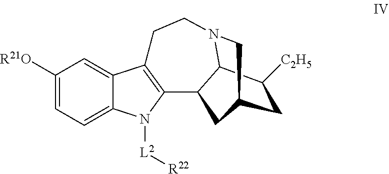

or a pharmaceutically acceptable salt and/or solvate thereof,

wherein

R²¹ is selected from the group consisting of hydrogen, a hydrolysable group selected from the group consisting of —C(O)R²³, —C(O)NR²⁴R²⁵ and —C(O)OR²⁶, where R²³ is selected from the group consisting of hydrogen, alkyl, substituted alkyl, alkenyl, substituted alkenyl, alkynyl and substituted alkynyl, R²⁴ and R²⁵ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic, R²⁶ is selected from the group consisting of alkyl, substituted alkyl, alkenyl, substituted alkenyl, alkynyl, substituted alkynyl, aryl, substituted aryl, heteroaryl, substituted heteroaryl, heterocyclic and substituted heterocyclic, provided that R²¹ is not a saccharide or an oligosaccharide;

L² is selected from the group consisting of a covalent bond and a cleavable linker group;

R²² is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic, provided that R is not a saccharide or an oligosaccharide;

provided that when L² is a covalent bond and R²² is hydrogen, then R²¹ is selected from the group consisting of —C(O)NR²⁴R²⁵ and —C(O)OR²⁶; and

further provided that when R²¹ is hydrogen or —C(O)R²³ and L² is a covalent bond, then R²² is not hydrogen.

In one embodiment, the noribogaine derivative is represented by Formula V:

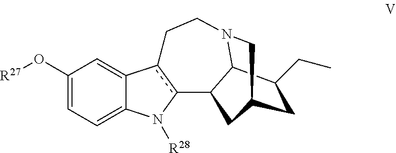

or a pharmaceutically acceptable salt and/or solvate thereof,
wherein:

[symbol] refers to a single or a double bond provided that when [symbol] is a single bond, Formula V refers to the corresponding dihydro compound;

R²⁷ is hydrogen or SO₂OR²⁹;

R²⁸ is hydrogen or SO₂OR²⁹;

R²⁹ is hydrogen or C₁-C₆ alkyl;

provided that at least one of R²⁷ and R²⁸ is not hydrogen.

In one embodiment, the noribogaine derivative is represented by Formula VI:

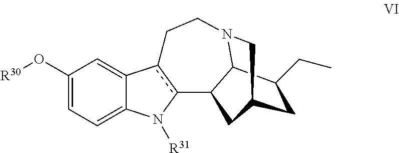

or a pharmaceutically acceptable salt and/or solvate thereof,
wherein:

[symbol] refers to a single or a double bond provided that when [symbol] is a single bond, Formula VI refers to the corresponding vicinal dihydro compound;

R³⁰ is hydrogen, a monophosphate, a diphosphate or a triphosphate; and

R³¹ is hydrogen, a monophosphate, a diphosphate or a triphosphate;

provided that both R³⁰ and R³¹ are not hydrogen;

wherein one or more of the monophosphate, diphosphate and triphosphate groups of R³⁰ and R³¹ are optionally esterified with one or more C₁-C₆ alkyl esters.

Noribogaine as utilized herein, can be replaced by a noribogaine derivative or a salt of noribogaine or the noribogaine derivative or a solvate of each of the foregoing.

In a preferred embodiment, the compound utilized herein is noribogaine or a salt thereof. In a more preferred embodiment, the compound utilized herein is noribogaine.

### Methods of the Invention

As will be apparent to the skilled artisan upon reading this disclosure, this invention provides a method for treating anxiety disorder, impulse control disorder, anger/violence-related disorders, or regulating food intake in a patient in need thereof, comprising administering to the patient a therapeutically effective amount of noribogaine, a noribogaine derivative, or a pharmaceutically acceptable salt and/or solvate thereof. In a preferred embodiment, the patient is not addicted to cocaine or an opiate. Noribogaine derivatives include, but are not limited to, the compounds described in the “Compositions” section above.

The following description of anxiety disorders and impulse control disorders is provided for the purpose of facilitating an understanding of the utility of the compounds and compositions of this invention. Disorders associated with violence and/or anger are included in these descriptions. The definitions of anxiety disorders and impulse control disorders given below are those listed in American Psychiatric Association, 2013, American Psychiatric Association, 1994a, or American Psychiatric Association, 1987. Additional information regarding these disorders can be found in this reference, as well as other references cited below, all of which are hereby incorporated herein by reference.

Anxiety disorders include panic disorder, agoraphobia with or without history of panic disorder, specific phobia, social phobia, obsessive-compulsive disorder, post-traumatic stress disorder, acute stress disorder and generalized anxiety disorder. It is contemplated that the compounds of this invention will be effective in treating these disorders in patients who have been diagnosed as having such disorders.

This invention provides for a method of treating a patient suffering from anxiety which comprises administering to the patient an amount of any of the compounds described herein effective to treat the subject's anxiety.

It is contemplated that the compounds described herein will be effective in treating obsessions and compulsions in patients who have been diagnosed as having obsessive compulsive disorder based upon administration of appropriate tests, which may include, but are not limited to any of the following: Yale Brown Obsessive Compulsive Scale (YBOCS) (for adults), National Institute of Mental Health Global OCD Scale (NIMH GOCS), CGI-Severity of Illness scale. It is further contemplated that the compounds described herein will be effective in inducing improvements in certain of the factors measured in these tests, such as a reduction of several points in the YBOCS total score. It is also contemplated that the compounds described herein will be effective in preventing relapse of obsessive compulsive disorder.

This invention provides a method of treating obsessions and compulsions in a patient with obsessive compulsive disorder, which comprises administering to the patient a therapeutically effective amount of any of the compounds utilized herein effective to treat the subject's obsessions and compulsions.

It is contemplated that the compounds described herein will be effective in treating panic disorder in patients who have been diagnosed with panic disorder on the basis of frequency of occurrence of panic attacks, or by means of the CGI-Severity of Illness scale. It is further contemplated that the compounds described herein will be effective in inducing improvements in certain of the factors measured in these evaluations, such as a reduction in frequency or elimination of panic attacks, an improvement in the CGI-Severity of Illness scale or a CGI-Global Improvement score of 1 (very much improved), 2 (much improved) or 3 (minimally improved). It is also contemplated that the compounds described herein will be effective in preventing relapse of panic disorder.

This invention provides a method of treating panic disorder, with or without agoraphobia, in a subject, which comprises administering to the patient a therapeutically effective amount of any of the compounds utilized herein to treat the subject's panic disorder.

It is contemplated that the compounds described herein can be effective in treating social anxiety disorder in patients who have been diagnosed as having social anxiety disorder based upon the administration of any of the following tests: the Liebowitz Social Anxiety Scale (LSAS), the CGI-Severity of Illness scale, the Hamilton Rating Scale for Anxiety (HAM-A), the Hamilton Rating Scale for Depression (HAM-D), the axis V Social and Occupational Functioning Assessment Scale of DSM-IV, the axis II (ICD-10) World Health Organization Disability Assessment, Schedule 2 (DAS-2), the Sheehan Disability Scales, the Schneier Disability Profile, the World Health Organization Quality of Life-100 (WHOQOL-100), or other tests as described in Bobes, 1998, which is incorporated herein by reference. It is further contemplated that the compounds described herein will be effective in inducing improvements as measured by these tests, such as the a change from baseline in the Liebowitz Social Anxiety Scale (LSAS), or a CGI-Global Improvement score of 1 (very much improved), 2 (much improved) or 3 (minimally improved). It is also contemplated that the compounds described herein will be effective in preventing relapse of social anxiety disorder.

This invention provides a method of treating social anxiety disorder in a patient which comprises administering to the patient a therapeutically effective amount of any of the compounds utilized herein to treat the subject's social anxiety disorder.

It is contemplated that the compounds utilized herein can be effective in treating generalized anxiety disorder in patients who have been diagnosed as having this disorder based upon the diagnostic criteria described in DSM-IV or DSM-5. It is further contemplated that the compounds utilized herein will be effective in reducing symptoms of this disorder, such as the following: excessive worry and anxiety, difficulty controlling worry, restlessness or feeling keyed up or on edge, being easily fatigued, difficulty concentrating or mind going blank, irritability, muscle tension, or sleep disturbance. It is also contemplated that the compounds described herein will be effective in preventing relapse of general anxiety disorder.

The invention provides a method of treating generalized anxiety disorder in a subject, which comprises administering to the patient an amount of any of the compounds described herein effective to treat the subject's generalized anxiety disorder.

Impulse control disorders include pathological gambling (PG), kleptomania, trichotillomania (TTM), intermittent explosive disorder (IED), and pyromania. Impulse control disorders may also include pathological skin picking (PSP), compulsive sexual behavior (CSB), compulsive buying (CB), conduct disorder, antisocial personality disorder, oppositional defiant disorder, borderline personality disorder, attention deficit/hyperactivity disorder (ADHD, which includes attention deficit disorder, ADD), schizophrenia, mood disorders, paraphilia, and internet addiction. Symptoms of impulse control disorders include: repetitive participation in behavior despite adverse consequences, diminished control over the behavior, an urge/impulse to engage in the behavior, and feelings of pleasure while participating in the behavior.

It is contemplated that the compounds utilized herein can be effective in treating impulse control disorders in patients who have at least one impulse control disorder based upon the diagnostic criteria described in DSM-IV or DSM-5. It is further contemplated that the compounds utilized herein will be effective in reducing symptoms of this disorder, including impulsivity or lack of self-control. It is also contemplated that the compounds described herein will be effective in preventing relapse of the impulse control disorder.

It is contemplated that the compounds utilized herein can be effective in treating ADHD or ADD in patients who have the disorder, based upon the diagnostic criteria described in DSM-IV or DSM-5. It is further contemplated that the compounds utilized herein will be effective in reducing symptoms of this disorder, including impulsivity or lack of self-control. It is also contemplated that the compounds described herein will be effective in preventing relapse of ADD or ADHD.

It is contemplated that the compounds utilized herein can be effective in treating schizophrenia in patients who have the disorder, based upon the diagnostic criteria described in DSM-IV or DSM-5. Schizophrenia is characterized by delusions, hallucinations, disorganized speech and behavior, and other symptoms that cause social or occupational dysfunction. It is further contemplated that the compounds utilized herein will be effective in reducing symptoms of this disorder. It is also contemplated that the compounds described herein will be effective in preventing relapse of schizophrenia.

It is contemplated that the compounds described herein will be effective in treating non-suicidal self injury disorder in patients who have been diagnosed with this disorder based on the patient's exhibition of symptoms including deliberate tissue injury without suicidal intent (e.g., cutting, burning, self-poisoning, or self-mutilation). It is further contemplated that the compounds described herein will be effective in inducing improvements in certain of these factors, such as a reduction in frequency or elimination of self injury. It is also contemplated that the compounds described herein will be effective in preventing relapse of non-suicidal self injury disorder.

This invention provides a method of treating non-suicidal self injury disorder in a subject, which comprises administering to the patient a therapeutically effective amount of any of the compounds utilized herein to treat the subject's non-suicidal self injury disorder.

It is contemplated that the compounds described herein will be effective in treating Münchausen syndrome in patients who have been diagnosed with this disorder based on the patient's propensity for feigning disease, illness, or psychological trauma to draw attention, sympathy, or reassurance to themselves. Symptoms may include frequent hospitalizations, knowledge of several illnesses, frequent requests for medication (e.g., pain killers), willingness to undergo extensive surgery, few to no visitors during hospitalizations, and exaggerated or fabricated stories about multiple medical problems. It is further contemplated that the compounds described herein will be effective in inducing improvements in certain of these factors, such as a reduction in frequency or elimination of one or more symptoms. It is also contemplated that the compounds described herein will be effective in preventing relapse of Münchausen syndrome. Münchausen syndrome also includes Münchausen syndrome by proxy, in which a caregiver exaggerates, fabricates, or induces illness in someone in his/her care.

This invention provides a method of treating Münchausen syndrome in a subject, which comprises administering to the patient a therapeutically effective amount of any of the compounds utilized herein to treat the subject's Münchausen syndrome.

It is contemplated that the compounds described herein will be effective in treating disruptive mood dysregulation disorder in patients who have been diagnosed with this disorder on the basis of severe and recurrent temper outbursts, grossly out of proportion to the stimulus or situation, as well as a persistent irritable/angry mood most of the time. It is further contemplated that the compounds described herein will be effective in inducing improvements in certain of these factors, such as a reduction in frequency or elimination of tember outbursts and/or an improvement in mood. It is also contemplated that the compounds described herein will be effective in preventing relapse of disruptive mood dysregulation disorder disorder.

This invention provides a method of treating disruptive mood dysregulation disorder in a subject, which comprises administering to the patient a therapeutically effective amount of any of the compounds utilized herein to treat the subject's disruptive mood dysregulation disorder.

It is contemplated that the compounds utilized herein can be effective in reducing the frequency, intensity, and duration of anger and/or violence in individuals prone to one or both. Although anger and violence disorders other than those associated with other disorders (e.g., as described above) are not outlined in DSM IV or DSM 5, many health professionals recognize that such disorders are associated with significant dysfunction. Anger management training and other psychosocial treatments are often used in an effort to treat these individuals.

It is contemplated that the compounds utilized herein can be effective in regulating food intake and/or reducing food cravings in patients in need thereof. In some embodiments, the patient is overweight. In some embodiments, the patient is obese. In some embodiments, the patient exhibits comorbidities associated with overweight/obesity, for example coronary heart disease, high blood pressure, stroke, type 2 diabetes, abnormal levels of blood fats, metabolic syndrome, cancer, osteoarthritis, sleep apnea, reproductive issues, and/or gallstones.

In a preferred embodiment, the invention provides a method for treating anxiety disorders, impulse control disorders, OCD, and/or anger/violence-related disorders, or regulating food intake and/or food cravings, in a patient in need thereof, comprising administering to the patient a therapeutically effective amount of noribogaine, noribogaine derivative, or a pharmaceutically acceptable salt and/or solvate thereof, wherein the patient is not addicted to cocaine or an opiate, and further wherein the therapeutically effective amount provides average noribogaine serum levels of between about 50 to about 180 ng/ml. In some embodiments, the average noribogaine serum level provided by the dosage is less than about 50 ng/mL. In one embodiment, the therapeutically effective amount is between about 1 mg to about 4 mg per kg of body weight. In one embodiment, the therapeutically effective amount is between about 50 ng to about 100 μg per kg of body weight. In one embodiment, an anxiety disorder is treated. In one embodiment, OCD is treated. In one embodiment, an impulse control disorder is treated. On one embodiment, an anger-related disorder is treated. in one embodiment, a violence-related disorder is treated. In one embodiment, symptoms of anger are reduced or eliminated. In one embodiment, violent outbursts are reduced or eliminated. In one embodiment, food intake is regulated. In one embodiment, food cravings are attenuated. In one embodiment, the noribogaine, noribogaine derivative, or pharmaceutically acceptable salt and/or solvate thereof is administered by sublingual, intranasal, or intrapulmonary delivery.

### Dosage and Routes of Administration

In some embodiments, the composition is administered via sublingual, intranasal, or intrapulmonary delivery. In one aspect, the invention provides administering a pharmaceutical composition comprising a pharmaceutically effective amount of noribogaine and a pharmaceutically acceptable excipient, wherein the therapeutically effective amount of noribogaine is an amount that delivers an aggregate amount of noribogaine of about 50 ng to about 100 μg per kg body weight per day. In some aspects, the therapeutically effective amount of noribogaine is an amount that delivers an aggregate amount of noribogaine of about 50 ng to about 50 μg per kg body weight per day. In some aspects, the therapeutically effective amount of noribogaine is an amount that delivers an aggregate amount of noribogaine of about 50 ng to about 10 μg per kg body weight per day. In some aspects, the therapeutically effective amount of noribogaine is an amount that delivers an aggregate amount of noribogaine of about 50 ng to about 1 μg per kg body weight per day. In some aspects, the composition is administered once per day. In some aspects, the composition is administered two or more times per day. In some embodiments, the composition is administered less than once a day, for example once every two days, once every three days, once every four days, once a week, etc.

In some embodiments, the composition is administered via oral, buccal, transdermal, internal, pulmonary, rectal, nasal, vaginal, lingual, intravenous, intraarterial, intramuscular, intraperitoneal, intracutaneous or subcutaneous delivery.

In one embodiment, the dosage or aggregate dosage of compound is from about 1 mg to about 4 mg per kg body weight per day. The aggregate dosage is the combined dosage, for example the total amount of noribogaine, noribogaine derivative, or pharmaceutically acceptable salt and/or solvate thereof administered over a 24-hour period where smaller amounts are administered more than once per day.

In one embodiment, the dosage or aggregate dosage of noribogaine, noribogaine derivative, or salt or solvate thereof is from about 1 mg/kg to about 4 mg/kg body weight per day. The aggregate dosage is the combined dosage, for example the total amount of noribogaine, noribogaine derivative, or pharmaceutically acceptable salt or solvate thereof administered over a 24-hour period where smaller amounts are administered more than once per day. In one embodiment, the dosage or aggregate dosage of noribogaine, noribogaine derivative, or salt or solvate thereof is from about 1.3 mg/kg to about 4 mg/kg body weight. In one embodiment, the dosage or aggregate dosage of noribogaine, noribogaine derivative, or salt or solvate thereof is from about 1.3 mg/kg to about 3 mg/kg body weight. In one embodiment, the dosage or aggregate dosage of noribogaine, noribogaine derivative, or salt or solvate thereof is from about 1.3 mg/kg to about 2 mg/kg body weight. In one embodiment, the dosage or aggregate dosage of noribogaine, noribogaine derivative, or salt or solvate thereof is from about 1.5 mg/kg to about 3 mg/kg body weight. In one embodiment, the dosage or aggregate dosage of noribogaine, noribogaine derivative, or salt or solvate thereof is from about 1.7 mg/kg to about 3 mg/kg body weight. In one embodiment, the dosage or aggregate dosage of noribogaine, noribogaine derivative, or salt or solvate thereof is from about 2 mg/kg to about 4 mg/kg body weight. In one embodiment, the dosage or aggregate dosage of noribogaine, noribogaine derivative, or salt or solvate thereof is from about 2 mg/kg to about 3 mg/kg body weight. In one embodiment, the dosage or aggregate dosage of noribogaine, noribogaine derivative, or salt or solvate thereof is about 2 mg/kg body weight. The ranges include both extremes as well as any subranges there between.

In one embodiment, the dosage or aggregate dosage of noribogaine, noribogaine derivative, or salt or solvate thereof is about 4 mg/kg body weight per day. In one embodiment, the dosage or aggregate dosage of noribogaine, noribogaine derivative, or salt or solvate thereof is about 3 mg/kg body weight per day. In one embodiment, the dosage or aggregate dosage of noribogaine, noribogaine derivative, or salt or solvate thereof is about 2 mg/kg body weight per day. In one embodiment, the dosage or aggregate dosage of noribogaine, noribogaine derivative, or salt or solvate thereof is about 1.9 mg/kg body weight per day. In one embodiment, the dosage or aggregate dosage of noribogaine, noribogaine derivative, or salt or solvate thereof is about 1.8 mg/kg body weight per day. In one embodiment, the dosage or aggregate dosage of noribogaine, noribogaine derivative, or salt or solvate thereof is about 1.7 mg/kg body weight per day. In one embodiment, the dosage or aggregate dosage of noribogaine, noribogaine derivative, or salt or solvate thereof is about 1.6 mg/kg body weight per day. In one embodiment, the dosage or aggregate dosage of noribogaine, noribogaine derivative, or salt or solvate thereof is about 1.5 mg/kg body weight per day. In one embodiment, the dosage or aggregate dosage of noribogaine, noribogaine derivative, or salt or solvate thereof is about 1.4 mg/kg body weight per day. In one embodiment, the dosage or aggregate dosage of noribogaine, noribogaine derivative, or salt or solvate thereof is about 1.3 mg/kg body weight per day. In one embodiment, the dosage or aggregate dosage of noribogaine, noribogaine derivative, or salt or solvate thereof is about 1.2 mg/kg body weight per day. In one embodiment, the dosage or aggregate dosage of noribogaine, noribogaine derivative, or salt or solvate thereof is about 1.1 mg/kg body weight per day. In one embodiment, the dosage or aggregate dosage of noribogaine, noribogaine derivative, or salt or solvate thereof is about 1 mg/kg body weight per day.

In one embodiment, the dosage or aggregate dosage of noribogaine, noribogaine derivative, or salt or solvate thereof is between about 70 mg and about 150 mg. In one embodiment, the dosage or aggregate dosage of noribogaine, noribogaine derivative, or salt or solvate thereof is between about 75 mg and about 150 mg. In one embodiment, the dosage or aggregate dosage of noribogaine, noribogaine derivative, or salt or solvate thereof is between about 80 mg and about 140 mg. In one embodiment, the dosage or aggregate dosage of noribogaine, noribogaine derivative, or salt or solvate thereof is between about 90 mg and about 140 mg. In one embodiment, the dosage or aggregate dosage of noribogaine, noribogaine derivative, or salt or solvate thereof is between about 90 mg and about 130 mg. In one embodiment, the dosage or aggregate dosage of noribogaine, noribogaine derivative, or salt or solvate thereof is between about 100 mg and about 130 mg. In one embodiment, the dosage or aggregate dosage of noribogaine, noribogaine derivative, or salt or solvate thereof is between about 110 mg and about 130 mg.

In one embodiment, the average serum concentration of noribogaine is from about 50 ng/mL to about 180 ng/mL, or about 60 ng/mL to about 180 ng/mL. In one embodiment, the average serum concentration of noribogaine is from about 50 ng/mL to about 150 ng/mL, or about 60 ng/mL to about 150 ng/mL. In one embodiment, the average serum concentration of noribogaine is from about 50 ng/mL to about 100 ng/mL, or about 60 ng/mL to about 100 ng/mL. In one embodiment, the average serum concentration of noribogaine is from about 80 ng/mL to about 150 ng/mL. In one embodiment, the average serum concentration of noribogaine is from about 80 ng/mL to about 100 ng/mL. The ranges include both extremes as well as any subranges between.

In one embodiment, the dosage of noribogaine, noribogaine derivative, or pharmaceutically acceptable salt or solvate thereof provides a serum concentration of between about 1000 ng*hr/mL and about 6000 ng*hr/mL (AUC/24 h). In one embodiment, the dosage of noribogaine, noribogaine derivative, or pharmaceutically acceptable salt or solvate thereof provides a serum concentration of between about 1200 ng*hr/mL and about 5800 ng*hr/mL (AUC/24 h). In one embodiment, the dosage of noribogaine, noribogaine derivative, or pharmaceutically acceptable salt or solvate thereof provides a serum concentration of between about 1200 ng*hr/mL and about 5500 ng*hr/mL (AUC/24 h). The ranges include both extremes as well as any subranges between.

In one embodiment, the dosage of noribogaine, noribogaine derivative, or pharmaceutically acceptable salt or solvate thereof provides a maximum serum concentration (Cmax) of less than about 250 ng/mL. In one embodiment, the dosage of noribogaine, noribogaine derivative, or pharmaceutically acceptable salt or solvate thereof provides a Cmax between about 40 ng/mL and about 250 ng/mL. In a preferred embodiment, the dosage of noribogaine, noribogaine derivative, or pharmaceutically acceptable salt or solvate thereof provides a Cmax between about 60 ng/mL and about 200 ng/mL. In one embodiment, the dosage of noribogaine, noribogaine derivative, or pharmaceutically acceptable salt or solvate thereof provides a Cmax between about 100 ng/mL and about 180 ng/mL.

In some embodiments, the patient is administered periodically, such as once, twice, three time, four times or five time daily with noribogaine, noribogaine derivative, or salt and/or solvate thereof. In some embodiments, the administration is once daily, or once every second day, once every third day, three times a week, twice a week, or once a week. The dosage and frequency of the administration depends on the route of administration, content of composition, age and body weight of the patient, condition of the patient, without limitation. Determination of dosage and frequency suitable for the present technology can be readily made by a qualified clinician.

In another embodiment, there is provided a unit dose of noribogaine, noribogaine derivative, or salt or solvate thereof which is about 50 mg to about 200 mg per dose. In one embodiment, the unit dose is about 50 to about 120 mg per dose. In one embodiment, the unit dose is about 120 mg per dose. It being understood that the term “unit dose” means a dose sufficient to provide therapeutic results whether given all at once or serially over a period of time.

In some embodiments, the patient is administered an initial dose of noribogaine, noribogaine derivative, or pharmaceutically acceptable salt or solvate thereof, followed by one or more additional doses. In one embodiment, such a dosing regimen provides an average serum concentration of noribogaine of about 50 ng/mL to about 180 ng/mL. In one embodiment, the one or more additional doses maintain an average serum concentration of about 50 ng/mL to about 180 ng/mL over a period of time.

In some embodiments, the initial dose of noribogaine, noribogaine derivative, or salt or solvate thereof is from about 75 mg to about 120 mg. In one embodiment, the initial dose is about 75 mg. In one embodiment, the initial dose is about 80 mg. In one embodiment, the initial dose is about 85 mg. In one embodiment, the initial dose is about 90 mg. In one embodiment, the initial dose is about 95 mg. In one embodiment, the initial dose is about 100 mg. In one embodiment, the initial dose is about 105 mg. In one embodiment, the initial dose is about 110 mg. In one embodiment, the initial dose is about 115 mg. In one embodiment, the initial dose is about 120 mg.

In some embodiments, the one or more additional doses are lower than the initial dose. In one embodiment, the one or more additional doses are from about 5 mg to 5 about 0 mg. In one embodiment, the one or more additional doses may or may not comprise the same amount of noribogaine, noribogaine derivative, or salt or solvate thereof. In one embodiment, at least one additional dose is about 5 mg. In one embodiment, at least one additional dose is about 10 mg. In one embodiment, at least one additional dose is about 15 mg. In one embodiment, at least one additional dose is about 20 mg. In one embodiment, at least one additional dose is about 25 mg. In one embodiment, at least one additional dose is about 30 mg. In one embodiment, at least one additional dose is about 35 mg. In one embodiment, at least one additional dose is about 40 mg. In one embodiment, at least one additional dose is about 45 mg. In one embodiment, at least one additional dose is about 50 mg.

In one embodiment, the one or more additional doses are administered periodically. In one embodiment, the one or more additional doses are administered approximately every 4 hours. In one embodiment, the one or more additional doses are administered every 6 hours. In one embodiment, the one or more additional doses are administered approximately every 8 hours. In one embodiment, the one or more additional doses are administered approximately every 10 hours. In one embodiment, the one or more additional doses are administered approximately every 12 hours. In one embodiment, the one or more additional doses are administered approximately every 18 hours. In one embodiment, the one or more additional doses are administered approximately every 24 hours. In one embodiment, the one or more additional doses are administered approximately every 36 hours. In one embodiment, the one or more additional doses are administered approximately every 48 hours.

In one aspect, this invention relates to a method for attenuating symptoms of anxiety disorder, impulse control disorder, or an anger and/or violence-related disorder in a human patient, comprising administering to the patient a dosage of noribogaine or pharmaceutically acceptable salt and/or solvate thereof that provides an average serum concentration of about 50 ng/mL to about 180 ng/mL, said concentration being sufficient to attenuate said symptoms while maintaining a QT interval of less than about 500 ms during said treatment. In some embodiments, the concentration is sufficient to attenuate said symptoms while maintaining a QT interval of less than about 470 ms during treatment. Preferably, the concentration is sufficient to attenuate said symptoms while maintaining a QT interval of less than about 450 ms during treatment. In one embodiment, the concentration is sufficient to attenuate said symptoms while maintaining a QT interval of less than about 420 ms during treatment.

In one aspect, this invention relates to a method for attenuating food cravings in a human patient, comprising administering to the patient a dosage of noribogaine or pharmaceutically acceptable salt and/or solvate thereof that provides an average serum concentration of about 50 ng/mL to about 400 ng/mL, said concentration being sufficient to attenuate said cravings while maintaining a QT interval of less than about 500 ms during said treatment. In some embodiments, the concentration is sufficient to attenuate said cravings while maintaining a QT interval of less than about 470 ms during treatment. Preferably, the concentration is sufficient to attenuate said cravings while maintaining a QT interval of less than about 450 ms during treatment. In one embodiment, the concentration is sufficient to attenuate said cravings while maintaining a QT interval of less than about 420 ms during treatment.

In one embodiment, the QT interval is not prolonged more than about 50 ms. In one embodiment, the QT interval is not prolonged more than about 40 ms. In one embodiment, the QT interval is not prolonged more than about 30 ms. In a preferred embodiment, the QT interval is not prolonged more than about 20 ms. In one embodiment, the QT interval is not prolonged more than about 10 ms.

The compositions, provided herein or known, suitable for administration in accordance with the methods provide herein, can be suitable for a variety of delivery modes including, without limitation, oral and transdermal delivery. Compositions suitable for internal, pulmonary, rectal, nasal, vaginal, lingual, intravenous, intraarterial, intramuscular, intraperitoneal, intracutaneous and subcutaneous routes may also be used. A particularly suitable composition comprises a composition suitable for a transdermal route of delivery in which the noribogaine or noribogaine derivative is applied as part of a cream, gel or, preferably, patch (for examples of transdermal formulations, see U.S. Pat. Nos. 4,806,341; 5,149,538; and 4,626,539, each of which are incorporated herein by reference). Other dosage forms include tablets, capsules, pills, powders, aerosols, suppositories, parenterals, and oral liquids, including suspensions, solutions and emulsions. Sustained release dosage forms may also be used. All dosage forms may be prepared using methods that are standard in the art (see e.g., Remington's Pharmaceutical Sciences, 16th ed., A. Oslo editor, Easton Pa. 1980).

Noribogaine, a noribogaine derivative, or a pharmaceutically acceptable salt and/or solvate thereof can also be used in conjunction with any of the vehicles and excipients commonly employed in pharmaceutical preparations, e.g., talc, gum Arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions containing noribogaine may be prepared using conventional techniques that may include sterile isotonic saline, water, 1,3-butanediol, ethanol, 1,2-propylene glycol, polyglycols mixed with water, Ringer's solution, etc.

### Patient Pre-Screening and Monitoring

Pre-screening of patients before treatment with noribogaine and/or monitoring of patients during noribogaine, noribogaine derivative, or pharmaceutically acceptable sald and/or solvate thereof treatment may be required to ensure that QT interval is not prolonged beyond a certain value. For example, QT interval greater than about 500 ms can be considered dangerous for individual patients. Pre-screening and/or monitoring may be necessary at high levels of noribogaine treatment.

In a preferred embodiment, a patient receiving a therapeutic dose of noribogaine is monitored in a clinical setting. Monitoring may be necessary to ensure the QT interval is not prolonged to an unacceptable degree. A “clinical setting” refers to an inpatient setting (e.g., inpatient clinic, hospital, rehabilitation facility) or an outpatient setting with frequent, regular monitoring (e.g., outpatient clinic that is visited daily to receive dose and monitoring).

Monitoring includes monitoring of QT interval. Methods for monitoring of QT interval are well-known in the art, for example by ECG.

In one embodiment, a patient receiving a maintenance dose of noribogaine is not monitored in a clinical setting. In one embodiment, a patient receiving a maintenance dose of noribogaine is monitored periodically, for example daily, weekly, monthly, or occasionally.

In one aspect, this invention relates to a method for treating, preventing, or attenuating a disease or disorder or symptoms of a disease or disorder described herein who is prescreened to evaluate the patient's expected tolerance for prolongation of QT interval, administering to the patient a dosage of noribogaine, noribogaine derivative, or pharmaceutically acceptable salt or solvate thereof that provides an average serum concentration of about 50 ng/mL to about 180 ng/mL, said concentration being sufficient to inhibit or ameliorate said abuse or symptoms while maintaining a QT interval of less than about 500 ms during said treatment. In some embodiments, the concentration is sufficient to attenuate said abuse or symptoms while maintaining a QT interval of less than about 470 ms during treatment. Preferably, the concentration is sufficient to attenuate said abuse or symptoms while maintaining a QT interval of less than about 450 ms during treatment. In one embodiment, the concentration is sufficient to attenuate said abuse or symptoms while maintaining a QT interval of less than about 420 ms during treatment.

In one embodiment, prescreening of the patient comprises ascertaining that noribogaine treatment will not result in a maximum QT interval over about 500 ms. In one embodiment, prescreening of the patient comprises ascertaining that noribogaine treatment will not result in a maximum QT interval over about 470 ms. In one embodiment, prescreening comprises ascertaining that noribogaine treatment will not result in a maximum QT interval over about 450 ms. In one embodiment, prescreening comprises ascertaining that noribogaine treatment will not result in a maximum QT interval over about 420 ms. In one embodiment, prescreening comprises determining the patient's pre-treatment QT interval.

As it relates to pre-screening or pre-selection of patients, patients may be selected based on any criteria as determined by the skilled clinician. Such criteria may include, by way of non-limiting example, pre-treatment QT interval, pre-existing cardiac conditions, risk of cardiac conditions, age, sex, general health, and the like. The following are examples of selection criteria for disallowing noribogaine treatment or restricting dose of noribogaine administered to the patient: high QT interval before treatment (e.g., such that there is a risk of the patient's QT interval exceeding about 500 ms during treatment); congenital long QT syndrome; bradycardia; hypokalemia or hypomagnesemia; recent acute myocardial infarction; uncompensated heart failure; and taking other drugs that increase QT interval. In some embodiments, the methods can include selecting and/or administering/providing noribogaine to a patient that lacks one more of such criteria.

In one embodiment, this invention relates to pre-screening a patient to determine if the patient is at risk for prolongation of the QT interval beyond a safe level. In one embodiment, a patient at risk for prolongation of the QT interval beyond a safe level is not administered noribogaine. In one embodiment, a patient at risk for prolongation of the QT interval beyond a safe level is administered noribogaine at a limited dosage.

In one embodiment, this invention relates to monitoring a patient who is administered a therapeutic dose of noribogaine. In one embodiment, the dose of noribogaine is reduced if the patient has serious adverse side effects. In one embodiment, the noribogaine treatment is discontinued if the patient has serious adverse side effects. In one embodiment, the adverse side effect is a QT interval that is prolonged beyond a safe level. The determination of a safe level of prolongation is within the skill of a qualified clinician.

In one aspect, this invention relates to a method for treating an anxiety disorder, an impulse control disorder, or an anger/violence-related disorder, and/or treating or attenuating the symptoms thereof in a patient, comprising selecting a patient exhibiting symptoms of an anxiety disorder, impulse control disorder, or anger/violence-related disorder who is prescreened to evaluate the patient's expected tolerance for prolongation of QT interval, administering to the patient a dosage of noribogaine, noribogaine derivative, or pharmaceutically acceptable salt and/or solvate thereof that provides an average serum concentration of about 50 ng/mL to about 850 ng/mL, said concentration being sufficient to inhibit or ameliorate said disorder or symptoms while maintaining a QT interval of less than about 500 ms during said treatment. In some embodiments, the concentration is sufficient to attenuate said symptoms while maintaining a QT interval of less than about 470 ms during treatment. Preferably, the concentration is sufficient to attenuate said symptoms while maintaining a QT interval of less than about 450 ms during treatment. In one embodiment, the concentration is sufficient to attenuate said symptoms while maintaining a QT interval of less than about 420 ms during treatment.

In one aspect, this invention relates to a method for regulating food intake, and/or treating or attenuating food cravings, in a patient, comprising selecting an overweight or obese patient who is prescreened to evaluate the patient's expected tolerance for prolongation of QT interval, administering to the patient a dosage of noribogaine, noribogaine derivative, or pharmaceutically acceptable salt and/or solvate thereof that provides an average serum concentration of about 50 ng/mL to about 180 ng/mL, said concentration being sufficient to inhibit or ameliorate said disorder or symptoms while maintaining a QT interval of less than about 500 ms during said treatment.

### Kit of Parts

One aspect of this invention is directed to a kit of parts for the treatment a condition in a patient which is treatable with noribogaine, noribogaine derivative, or pharmaceutically acceptable salt or solvate thereof, wherein the kit comprises a composition comprising noribogaine, noribogaine derivative, or salt or solvate thereof and a means for administering the composition to a patient in need thereof. The means for administration to a patient can include, for example, any one or combination of a pharmaceutically acceptable formulation comprising noribogaine, or a noribogaine derivative, or a pharmaceutically acceptable salt or solvate thereof (e.g., a pill, transdermal patch, injectable, and the like, without limitation) and optionally a means for dispensing and/or administering the formulation (e.g., a syringe, a needle, an IV bag comprising the composition, a vial comprising the composition, an inhaler comprising the composition, etc, without limitation). In one embodiment, the kit of parts further comprises instructions for dosing and/or administration of the composition.

In some aspects, the invention is directed to a kit of parts for administration of noribogaine, the kit comprising multiple delivery vehicles, wherein each delivery vehicle contains a discrete amount of noribogaine and further wherein each delivery vehicle is identified by the amount of noribogaine provided therein; and optionally further comprising a dosing treatment schedule in a readable medium. In some embodiments, the dosing treatment schedule includes the amount of noribogaine required to achieve each average serum level is provided. In some embodiments, the kit of parts includes a dosing treatment schedule that provides an attending clinician the ability to select a dosing regimen of noribogaine based on the sex of the patient, mass of the patient, and the serum level that the clinician desires to achieve. In some embodiments, the dosing treatment schedule further provides information corresponding to the volume of blood in a patient based upon weight (or mass) and sex of the patient. In an embodiment, the storage medium can include an accompanying pamphlet or similar written information that accompanies the unit dose form in the kit. In an embodiment, the storage medium can include electronic, optical, or other data storage, such as a non-volatile memory, for example, to store a digitally-encoded machine-readable representation of such information.

The term “delivery vehicle” as used herein refers to any formulation that can be used for administration of noribogaine to a patient. Non-limiting, exemplary delivery vehicles include caplets, pills, capsules, tablets, powder, liquid, or any other form by which the drug can be administered. Delivery vehicles may be intended for administration by oral, inhaled, injected, or any other means.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

In some aspects, the machine-readable medium comprises software that contains information regarding dosing schedules for the unit dose form of noribogaine and optionally other drug information. In some embodiments, the software may be interactive, such that the attending clinician or other medical professional can enter patient information. In a non-limiting example, the medical professional may enter the weight and sex of the patient to be treated, and the software program provides a recommended dosing regimen based on the information entered. The amount and timing of noribogaine recommended to be delivered will be within the dosages that result in the serum concentrations as provided herein.

In some embodiments, the kit of parts comprises multiple delivery vehicles in a variety of dosing options. For example, the kit of parts may comprise pills or tablets in multiple dosages, such as 120 mg, 90 mg, 60 mg, 30 mg, 20 mg, 10 mg, and/or 5 mg of noribogaine per pill. Each pill is labeled such that the medical professional and/or patient can easily distinguish different dosages. Labeling may be based on printing or embossing on the pill, shape of the pill, color of pill, the location of the pill in a separate, labeled compartment within the kit, and/or any other distinguishing features of the pill. In some embodiments, all of the delivery vehicles within a kit are intended for one patient. In some embodiments, the delivery vehicles within a kit are intended for multiple patients.

One aspect of this invention is directed to a kit of parts for the treatment, prevention, or attenuation of a disease or disorder or symptoms of a disease or disorder described herein, wherein the kit comprises a unit dose form of noribogaine, noribogaine derivative, or salt or solvate thereof. The unit dose form provides a patient with an average serum level of noribogaine of from about 50 ng/mL to about 180 ng/mL or about 60 ng/mL to about 180 ng/mL. The unit dose form provides a patient with an average serum level of noribogaine of from about 50 ng/mL to about 800 ng/mL or about 60 ng/mL to about 800 ng/mL. In one embodiment, the unit dose form provides a patient with an average serum level of noribogaine of from about 50 ng/mL to about 400 ng/mL or about 60 ng/mL to about 400 ng/mL. In one embodiment, the unit dose form provides a patient with an average serum level of noribogaine of from 80 ng/mL to 100 ng/mL.

In some embodiments, the unit dose of noribogaine, noribogaine derivative, or pharmaceutically acceptable salt or solvate thereof is from 20 mg to 120 mg. In one embodiment, the unit dose is 20 mg. In one embodiment, the unit dose is 30 mg. In one embodiment, the unit dose is 40 mg. In one embodiment, the unit dose is 50 mg. In one embodiment, the unit dose is 60 mg. In one embodiment, the unit dose is 70 mg. In one embodiment, the unit dose is 80 mg. In one embodiment, the unit dose is 90 mg. In one embodiment, the unit dose is 100 mg. In one embodiment, the unit dose is 110 mg. In one embodiment, the unit dose is 120 mg.

In some embodiments, the unit dose form comprises one or multiple dosages to be administered periodically, such as once, twice, three times, four times or five times daily with noribogaine or its prodrug. In some embodiments, the administration is once daily, or once every second day, once every third day, three times a week, twice a week, or once a week. The dosage and frequency of the administration depends on criteria including the route of administration, content of composition, age and body weight of the patient, condition of the patient, sex of the patient, without limitation, as well as by the severity of the addiction. Determination of the unit dose form providing a dosage and frequency suitable for a given patient can readily be made by a qualified clinician.

In some embodiments, the initial unit dose and one or more additional doses of noribogaine, noribogaine derivative, or salt or solvate thereof are provided as one or multiple dosages to be administered periodically, such as once, twice, three times, four times or five times daily with noribogaine or its prodrug. In some embodiments, the administration is once daily, or once every second day, once every third day, three times a week, twice a week, or once a week. The dosage and frequency of the administration depends on criteria including the route of administration, content of composition, age and body weight of the patient, condition of the patient, sex of the patient, without limitation, as well as by the severity of the addiction. Determination of the unit dose form providing a dosage and frequency suitable for a given patient can readily be made by a qualified clinician.

In one aspect, provided herein is a kit of parts comprising two or more doses of noribogaine, noribogaine derivative, or pharmaceutically acceptable salt or solvate thereof, wherein the two or more doses comprise an amount of noribogaine, noribogaine derivative, or pharmaceutically acceptable salt or solvate thereof that is sufficient to maintain a serum concentration of 50 ng/mL to 180 ng/mL when administered to a patient.

In one embodiment, one dose comprises an initial dose of noribogaine, noribogaine derivative, or pharmaceutically acceptable salt or solvate thereof, said initial dose being sufficient to achieve a therapeutic serum concentration when administered to a patient; and at least one additional dose, said additional dose sufficient to maintain a therapeutic serum concentration when administered to a patient, wherein the therapeutic serum concentration is between 50 ng/mL and 180 ng/mL In another embodiment, the initial dose is from 75 mg to 120 mg. In another embodiment, the at least one additional dose is from 5 mg to 25 mg.

These dose ranges may be achieved by transdermal, oral, or parenteral administration of noribogaine, noribogaine derivative, or a pharmaceutically acceptable salt or solvate thereof in unit dose form. Such unit dose form may conveniently be provided in transdermal patch, tablet, caplet, liquid or capsule form. In certain embodiments, the noribogaine is provided as noribogaine HCl, with dosages reported as the amount of free base noribogaine. In some embodiments, the noribogaine HCl is provided in hard gelatin capsules containing only noribogaine HCl with no excipients. In some embodiments, noribogaine is provided in saline for intravenous administration.

### Formulations

This invention further relates to pharmaceutically acceptable formulations comprising a unit dose of noribogaine, noribogaine derivative, or pharmaceutically acceptable salt or solvate thereof, wherein the amount of noribogaine is sufficient to provide an average serum concentration of about 50 ng/mL to about 180 ng/mL when administered to a patient. In a preferred embodiment, the amount of noribogaine is sufficient to provide an average serum concentration of about 80 ng/mL to about 100 ng/mL when administered to a patient. In one embodiment, the amount of noribogaine, noribogaine derivative, or pharmaceutically acceptable salt thereof is an amount that delivers an aggregate amount of noribogaine of about 50 ng to about 10 μg per kg body weight per day.

In some embodiments, the unit dose of noribogaine is administered in one or more dosings.

This invention further relates to pharmaceutically acceptable formulations comprising a unit dose of noribogaine, noribogaine derivative, or pharmaceutically acceptable salt or solvate thereof, wherein the amount of noribogaine is sufficient to provide and/or maintain an average serum concentration of about 50 ng/mL to about 180 ng/mL when administered to a patient. In a preferred embodiment, the amount of noribogaine is sufficient to provide and/or maintain an average serum concentration of 80 ng/mL to 100 ng/mL when administered to a patient.

In some embodiments, the unit dose of noribogaine is administered in one or more dosings.

In one embodiment, the amount of noribogaine is sufficient to provide an average serum concentration of noribogaine from 50 ng/mL to 180 ng/mL, or 60 ng/mL to 180 ng/mL. In one embodiment, the amount of noribogaine is sufficient to provide an average serum concentration of noribogaine from 50 ng/mL to 150 ng/mL, or 60 ng/mL to 150 ng/mL. In one embodiment, the amount of noribogaine is sufficient to provide an average serum concentration of noribogaine from about 50 ng/mL to about 120 ng/mL, or about 60 ng/mL to about 120 ng/mL. In one embodiment, the amount of noribogaine is sufficient to provide an average serum concentration of noribogaine from about 50 ng/mL to about 100 ng/mL, or about 60 ng/mL to about 100 ng/mL. In one embodiment, the amount of noribogaine is sufficient to provide an average serum concentration of noribogaine from about 50 ng/mL to about 120 ng/mL, or about 60 ng/mL to about 120 ng/mL. In one embodiment, the amount of noribogaine is sufficient to provide an average serum concentration of noribogaine from about 50 ng/mL to about 100 ng/mL, or about 60 ng/mL to about 100 ng/mL. In one embodiment, the amount of noribogaine is sufficient to provide an average serum concentration of noribogaine from about 80 ng/mL to about 100 ng/mL. The ranges include both extremes as well as any subranges between.

In some embodiments, the initial unit dose of noribogaine, noribogaine derivative, or pharmaceutically acceptable salt or solvate thereof is from about 50 mg to about 120 mg. In one embodiment, the unit dose is about 50 mg. In one embodiment, the unit dose is about 55 mg. In one embodiment, the unit dose is 60 mg. In one embodiment, the unit dose is about 65 mg. In one embodiment, the unit dose is about 70 mg. In one embodiment, the unit dose is about 75 mg. In one embodiment, the unit dose is about 80 mg. In one embodiment, the unit dose is about 85 mg. In one embodiment, the unit dose is about 90 mg. In one embodiment, the unit dose is about 95 mg. In one embodiment, the unit dose is about 100 mg. In one embodiment, the unit dose is 105 mg. In one embodiment, the unit dose is about 110 mg. In one embodiment, the unit dose is about 115 mg. In one embodiment, the unit dose is about 120 mg.

In some embodiments, the at least one additional dose of noribogaine, noribogaine derivative, or pharmaceutically acceptable salt or solvate thereof is from 5 mg to 75 mg. In one embodiment, the unit dose is 5 mg. In one embodiment, the unit dose is 10 mg. In one embodiment, the unit dose is 15 mg. In one embodiment, the unit dose is 20 mg. In one embodiment, the unit dose is 25 mg. In one embodiment, the unit dose is 30 mg. In one embodiment, the unit dose is 35 mg. In one embodiment, the unit dose is 40 mg. In one embodiment, the unit dose is 45 mg. In one embodiment, the unit dose is 50 mg. In one embodiment, the unit dose is 55 mg. In one embodiment, the unit dose is 60 mg. In one embodiment, the unit dose is 65 mg. In one embodiment, the unit dose is 70 mg. In one embodiment, the unit dose is 75 mg.

In some embodiments, the formulation comprises a delivery vehicle, as described above. In one embodiment, the delivery vehicle comprises 5 mg to 120 mg noribogaine, noribogaine derivative, or pharmaceutically acceptable salt or solvate thereof.

In some embodiments, the formulation is a controlled release formulation. The term “controlled release formulation” includes sustained release and time-release formulations. Controlled release formulations are well-known in the art. These include excipients that allow for sustained, periodic, pulse, or delayed release of the drug. Controlled release formulations include, without limitation, embedding of the drug into a matrix; enteric coatings; microencapsulation; gels and hydrogels; implants; transdermal patches; and any other formulation that allows for controlled release of a drug.

In one embodiment, the amount of noribogaine is sufficient to provide an average serum concentration of noribogaine from about 50 ng/mL to about 180 ng/mL, or about 60 ng/mL to about 180 ng/mL. In one embodiment, the amount of noribogaine is sufficient to provide an average serum concentration of noribogaine from about 50 ng/mL to about 150 ng/mL, or about 60 ng/mL to about 150 ng/mL. In one embodiment, the amount of noribogaine is sufficient to provide an average serum concentration of noribogaine from about 50 ng/mL to about 120 ng/mL, or about 60 ng/mL to about 120 ng/mL. In one embodiment, the amount of noribogaine is sufficient to provide an average serum concentration of noribogaine from about 50 ng/mL to about 100 ng/mL, or about 60 ng/mL to about 100 ng/mL. In one embodiment, the amount of noribogaine is sufficient to provide an average serum concentration of noribogaine from about 80 ng/mL to about 100 ng/mL. The ranges include both extremes as well as any subranges between.

In some embodiments, the unit dose of noribogaine, noribogaine derivative, or pharmaceutically acceptable salt or solvate thereof is from about 20 mg to about 120 mg. In one embodiment, the unit dose is about 20 mg. In one embodiment, the unit dose is about 30 mg. In one embodiment, the unit dose is about 40 mg. In one embodiment, the unit dose is about 50 mg. In one embodiment, the unit dose is about 60 mg. In one embodiment, the unit dose is about 70 mg. In one embodiment, the unit dose is about 80 mg. In one embodiment, the unit dose is about 90 mg. In one embodiment, the unit dose is about 100 mg. In one embodiment, the unit dose is about 110 mg. In one embodiment, the unit dose is about 120 mg.

This invention further relates to pharmaceutically acceptable formulations comprising a unit dose of noribogaine, noribogaine derivative, or pharmaceutically acceptable salt and/or solvate thereof, wherein the amount of noribogaine is sufficient to provide an average serum concentration of about 50 ng/mL to about 850 ng/mL when administered to a patient. In a preferred embodiment, the amount of noribogaine is sufficient to provide an average serum concentration of about 50 ng/mL to about 400 ng/mL when administered to a patient.

In one embodiment, the amount of noribogaine is sufficient to provide an average serum concentration of noribogaine from about 50 ng/mL to about 800 ng/mL or about 60 ng/mL to about 800 ng/mL. In one embodiment, the amount of noribogaine is sufficient to provide an average serum concentration of noribogaine from about 50 ng/mL to about 700 ng/mL or about 60 ng/mL to about 700 ng/mL. In one embodiment, the amount of noribogaine is sufficient to provide an average serum concentration of noribogaine from about 50 ng/mL to about 600 ng/mL, or about 60 ng/mL to about 600 ng/mL. In a preferred embodiment, the amount of noribogaine is sufficient to provide an average serum concentration of noribogaine from about 50 ng/mL to about 500 ng/mL, or about 60 ng/mL to about 500 ng/mL. In one embodiment, the amount of noribogaine is sufficient to provide an average serum concentration of noribogaine from about 50 ng/mL to about 400 ng/mL, or about 60 ng/mL to about 400 ng/mL. In one embodiment, the amount of noribogaine is sufficient to provide an average serum concentration of noribogaine from about 50 ng/mL to about 300 ng/mL, or about 60 ng/mL to about 300 ng/mL. In one embodiment, the amount of noribogaine is sufficient to provide an average serum concentration of noribogaine from about 50 ng/mL to about 200 ng/mL, or about 60 ng/mL to about 200 ng/mL. In one embodiment, the amount of noribogaine is sufficient to provide an average serum concentration of noribogaine from about 50 ng/mL to about 100 ng/mL, or about 60 ng/mL to about 100 ng/mL. The ranges include both extremes as well as any subranges between.

In some embodiments, the formulation is designed for periodic administration, such as once, twice, three times, four times or five times daily with noribogaine, noribogaine derivative, or a pharmaceutically acceptable salt or solvate thereof. In some embodiments, the administration is once daily, or once every second day, once every third day, three times a week, twice a week, or once a week. The dosage and frequency of the administration depends on the route of administration, content of composition, age and body weight of the patient, condition of the patient, without limitation. Determination of dosage and frequency suitable for the present technology can be readily made a qualified clinician.

In some embodiments, the formulation designed for administration in accordance with the methods provide herein can be suitable for a variety of delivery modes including, without limitation, oral, transdermal, sublingual, buccal, intrapulmonary or intranasal delivery. Formulations suitable for internal, pulmonary, rectal, nasal, vaginal, lingual, intravenous, intra-arterial, intramuscular, intraperitoneal, intracutaneous and subcutaneous routes may also be used. Possible formulations include tablets, capsules, pills, powders, aerosols, suppositories, parenterals, and oral liquids, including suspensions, solutions and emulsions. Sustained release dosage forms may also be used. All formulations may be prepared using methods that are standard in the art (see e.g., Remington's Pharmaceutical Sciences, 16th ed., A. Oslo editor, Easton Pa. 1980).

In a preferred embodiment, the formulation is designed for oral administration, which may conveniently be provided in tablet, caplet, sublingual, liquid or capsule form. In certain embodiments, the noribogaine is provided as noribogaine HCl, with dosages reported as the amount of free base noribogaine. In some embodiments, the noribogaine HCl is provided in hard gelatin capsules containing only noribogaine HCl with no excipients.

Noribogaine or a noribogaine derivative can also be used in conjunction with any of the vehicles and excipients commonly employed in pharmaceutical preparations, e.g., talc, gum Arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions containing noribogaine may be prepared using conventional techniques that may include sterile isotonic saline, water, 1,3-butanediol, ethanol, 1,2-propylene glycol, polyglycols mixed with water, Ringer's solution, etc.

The compositions utilized herein may be formulated for aerosol administration, particularly to the respiratory tract and including intrapulmonary or intranasal administration. The compound will generally have a small particle size, for example of the order of 5 microns or less. Such a particle size may be obtained by means known in the art, for example by micronization. The active ingredient may be provided in a pressurized pack with a suitable propellant such as a chlorofluorocarbon (CFC), (for example, dichlorodifluoromethane, trichlorofluoromethane, or dichlorotetrafluoroethane), carbon dioxide or other suitable gases. The aerosol may conveniently also contain a surfactant such as lecithin. The dose of drug may be controlled by a metered valve. Alternatively, the active ingredients may be provided in the form of a dry powder, for example a powder mix of the compound in a suitable powder base such as lactose, starch, starch derivatives such as hydroxypropylmethyl cellulose and polyvinylpyrrolidine. In some embodiments, the powder carrier will form a gel in the nasal cavity. The powder composition may be presented in unit dose form, for example in capsules or cartridges, gelatin or blister packs, from which the powder may be administered by means of an inhaler.

The compositions utilized herein may be formulated for sublingual administration, for example as sublingual tablets. Sublingual tablets are designed to dissolve very rapidly. The formulations of these tablets contain, in addition to the drug, a limited number of soluble excipients, usually lactose and powdered sucrose, but sometimes dextrose and mannitol.

It has been discovered that noribogaine has a bitter taste to at least some patients. Accordingly, compositions for oral use (including sublingual, inhaled, and other oral formulations) may be formulated to utilize taste-masking technologies. A number of ways to mask the taste of bitter drugs are known in the art, including addition of sugars, flavors, sweeteners, or coatings; use of lipoproteins, vesicles, and/or liposomes; granulation; microencapsulation; numbing of taste buds; multiple emulsion; modification of viscosity; prodrug or salt formation; inclusion or molecular complexes; ion exchange resins; and solid dispersion. Any method of masking the bitterness of the compound of the invention may be used.

### EXAMPLES

The following Examples are intended to further illustrate certain embodiments of the disclosure and are not intended to limit its scope.

### Example 1

### Social Interaction Test (SIT)

Animals:

Male albino Sprague-Dawley rats (Taconic Farms, N.Y.) are housed in pairs under a 12 hr light dark cycle (lights on at 0700 hrs.) with free access to food and water.

Rats are allowed to acclimate to the animal care facility for 5 days and are housed singly for 5 days prior to testing. Animals are handled for 5 minutes per day. On the test day, weight matched pairs of rats (±5%), unfamiliar to each other, are given identical treatments and returned to their home cages. Animals are randomly divided into 5 treatment groups, with 5 pairs per group, and are given one of the following i.p. treatments: Test Compound (1, 2 or 4 mg/kg), vehicle (1 ml/kg) or chlordiazepoxide (5 mg/kg). Dosing is done 1 hour prior to testing. Rats are subsequently placed in a white perspex test box or arena (54×37×26 cm), whose floor is divided up into 24 equal squares, for 15 minutes. An air conditioner is used to generate background noise and to keep the room at approximately 74° F. All sessions are videotaped using a JVC camcorder (model GR-SZ1, Elmwood Park, N.J.) with either TDK (HG ultimate brand) or Sony 30 minute videocassettes. All sessions are conducted between 13:00 and 16:30 hours. Active social interaction, defined as grooming, sniffing, biting, boxing, wrestling, following and crawling over or under, is scored using a stopwatch (Sportsline model no. 226, 1/100 sec. discriminability). The number of episodes of rearing (animal completely raises up its body on its hind limbs), grooming (licking, biting, scratching of body), and face ishing (i.e. hands are moved repeatedly over face), and number of squares crossed are scored. Passive social interaction (animals are lying beside or on top of each other) is not scored. All behaviors are assessed later by an observer who is blind as to the treatment of each pair. At the end of each test, the box is thoroughly wiped with moistened paper towels.

Data Analysis:

The social interaction data (time interacting, rearing and squares crossed) are subjected to a randomized, one-way ANOVA and post hoc tests conducted using the Student-Newman-Keuls test. The data are subjected to a test of normality (Shapiro-Wilk test). The data are analyzed using the GBSTAT program, version 6.5 (Dynamics Microsystems, Inc., Silver Spring, Md., 1997).

### Example 2

### Regulation of Food Intake in Rats

### Animals

The objective of this study was to determine the effect of noribogaine on food consumption in the Sprague-Dawley rat. Eight food-maintained young adult, male Sprague-Dawley rats (300-325 g) from Harlan were used in this study. Upon arrival, the rats were assigned a unique identification numbers (tail marks). Animals were housed 2-3 per cage in suspended polycarbonate rat cages with filter paper covering mesh shelf and were acclimated for up to 7 days. All rats were examined, handled, and weighed prior to initiation of the study to assure adequate health and suitability. During the course of the study, 12/12 light/dark cycles were maintained. The room temperature was 20-23° C. with a relative humidity maintained 30-70%. Water was provided ad libitum for the duration of the study. Rats were single housed and food-restricted and maintained at 85% of the free-feeding age-matched control body weight. Food maintained control rats were also single-housed with body weight maintained.

### Test Compounds

Noribogaine (12.5, 25 and 50 mg/kg, converted to free base doses with a correction factor 1.12) was dissolved in 35% of the total required volume of 0.5% Tween 80 in 5% Dextrose. Suspension was stirred for at least 30 minutes. 1.5% methylcellulose was added to make up 65% of the total volume and the suspension was stirred again for at least 30 minutes. As a result, 12.5 mg/kg and 25 mg/kg doses were clear solutions and 50 mg/kg was a slightly cloudy suspension.

The mix of 0.5% Tween 80 in 5% Dextrose (35% of total volume) and 1.5% methylcellulose solution (65% of total volume) was used as compound vehicle treatment.

Vehicle and noribogaine were administered PO 2 hours prior to test at a dose volume of 5 ml/kg.

Varenicline (1.7 mg/kg) was dissolved in saline (0.9% NaCl) and administered IP 30 minutes prior to test. Dose volume of varenicline was 1 ml/kg. The formulation of varenicline (1.7 mg/kg) was a clear solution.

### Food Maintained Responding Tests

Apparatus:

Food-maintained responding training and tests took place in experimental chambers within sound-attenuating cubicles equipped with an exhaust fan (Med Associates, VT). Each chamber contained two response levers situated on one wall of the chamber. A stimulus light was located above each lever and a house light is located at the top of opposite wall. A pellet receptacle was situated between the two levers for delivery of food pellets (Bio-Serv's Dustless Precision Pellets #F0165, 45 mg).

Food Training and Self-Administration Procedures:

Animals were trained to lever press for food. Animals were first trained to respond for food pellets under a FR3, time-out 20 seconds schedule of reinforcement. After the completion of training and the establishment of stable baselines, the effects of noribogaine were assessed. Noribogaine or the reference compound varenicline (a nicotinic acetylcholine receptor partial agonist) were only administered when the animals exhibited responding at baseline levels (i.e. no less than 6 infusions and less than 20% variation in the mean number of reinforcers earned in 1-hour training over last three consecutive non-drug test days). Compound testing was performed on Wednesdays and Fridays, assuming baseline levels of self-administration behavior on Tuesdays and Thursdays.

### Study Design and Data Analysis

A within-subject design in which each rat received all treatments was applied with a Latin square test schedule. The six treatments which were blind to the experimenter were:
1. Saline
2. Varenicline 1.7 mg/kg
3. Vehicle (35% of 0.5% Tween-80 in 5% Dextrose and 65% of 1.5% methylcellulose)
4. Noribogaine 12.5 mg/kg
5. Noribogaine 25 mg/kg
6. Noribogaine 50 mg/kg

The data of nicotine infusions or food pellets obtained during test sessions were analyzed via repeated measure ANOVA followed by Fisher LSD post hoc comparisons where appropriate. Percentage of inactive lever presses were also analyzed with repeated measure ANOVA for non-specific behavioral effects. An effect is considered significant if P<0.05. Data are represented as the mean and standard error to the mean (s.e.m.). Statistical outliers fell beyond mean+/−(2× standard deviation) are removed from the analysis. With this criterion, 0-2 outliers were eliminated in different measures (see details in Section 7, Statistical Tables).

### Results

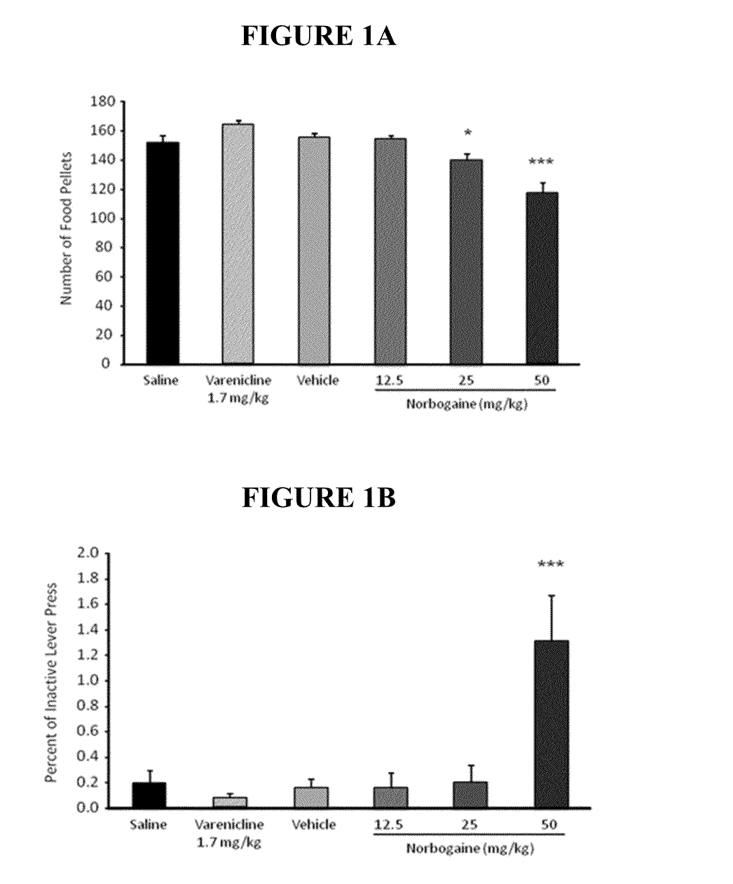

The effects of noribogaine and varenicline on food consumption in food maintained responding rats are shown in FIG. 1A. Repeated measure ANOVA found a significant main effect of treatment [F(5,33)=16.905, P<0.001]. Post hoc comparisons indicated that compared to vehicle treatment, noribogaine (25 and 50 mg/kg) significantly decreased food consumption (P<0.05 and P<0.001, respectively). Noribogaine (12.5 mg/kg) and varenicline (1.7 mg/kg) had no significant effect on this measure.

The effects of noribogaine and varenicline on inactive lever response in food maintained responding rats are shown in FIG. 1B. Repeated measure ANOVA found a significant main effect of treatment [F(5,31)=7.583, P<0.001]. This main effect is solely contributed by high dose noribogaine (50 mg/kg), as shown in post hoc comparisons (P<0.001 compared to vehicle treatment.) This result suggests that for high frequency lever-pressing in food test the high dose of noribogaine (50 mg/kg) may cause some behavioral disturbance.

At 12.5 mg/kg, noribogaine showed no effect on food consumption. At 25 mg/kg, noribogaine decreased food intake by 10% (P<0.05). At 50 mg/kg, noribogaine decreased food intake by 25% (P<0.001). In general, the inactive lever responding percentage was very low during food consumption test under all treatments.

### Example 3

### Single Dose Toxicity in Rats

The objective of this study was to determine the toxicity and toxicokinetic profile of noribogaine HCl following a single oral (gavage) administration in the Sprague-Dawley rat. A single dose of 100, 300 and 800 mg/kg (achieved with doses of 400 mg/kg 3 h+/−30 min apart because of the limitations of maximum dose formulation concentration). Five male rats/group were used. Mortality occurred in all male rats in the 800 mg/kg group, approximately 2-3 h after administration of the second dose of 400 mg/kg. Hypoactivity, vocalization, chewing movements, changes in respiration/posture, salivation, stimuli sensitivity, tremors, twitches and penile erection occurred prior to death. Hypoactivity, vocalization, salivation, stimuli sensitivity, loss of limb function and lying on the cage floor occurred on the day of treatment and persisted until Day 2 in 3/5 rats given 300 mg/kg. The low dose rats treated at 100 mg/kg did not show any treatment related signs. The NOAEL was determined to be 100 mg/kg.

### Example 4

### Single Dose Toxicity in Dogs

In an acute oral toxicity/TK study in dogs, no mortality occurred at doses of 5 (n=2) or 10 (n=2) mg/kg. Convulsions and other CNS-related clinical signs, including twitches, salivation, vocalization, incoordination and hypoactivity, occurred at a dose of 10 mg/kg, beginning 20 minutes after dosing and persisting until 3 h 40 m post-dose. The 5 mg/kg dose was considered the NOAEL, as only transient reduction in food consumption in one dog occurred at that dose.

### Example 5

### Single Dose Toxicity in Cynomolgus Monkeys

The objective of the study was to determine the toxicity and toxicokinetic profile of noribogaine following oral (gavage) administration to the cynomolgus monkey. Each dose was followed by a 7 day washout period. Dosing was staggered by 45 minutes. On study day 15, one animal was administered 80 mg/kg and the other animal was administered 160 mg/kg. The test article was administered as follows in Table 1:

TABLE 1
Toxicity and Toxicokinetic Study in Cynomolgus monkeys

| Treatment on Study Day | Dose Level (mg/kg) | Number of Animals |
|---|---|---|
| 1 | 20 | 2 males |
| 8 | 40 | 2 males |
| 15 | 80 and 160 | 2 males |

Parameters monitored on the study included: mortality, clinical signs and body weights. Blood samples were collected for TK evaluation. No mortality or treatment related clinical signs were noted for doses up to and including 160 mg/kg. The single dose maximum tolerated dose (MTD) was determined to be greater than 160 mg/kg based on the parameters monitored during the study.

### Example 6

### Fourteen Day Repeat Dose Toxicity and Toxicokinetics in Rats

This study was conducted to evaluate the toxicity profile of noribogaine-HCl following oral (gavage) administration to the rat for 14 days following Table 2 below:

TABLE 2
Toxicity and Toxicokinetic Study in Rats

| | | Dose Con- | Toxicology Animals | | | | Toxicokinetics | |
|---|---|---|---|---|---|---|---|---|
| | Dose Level | centration | Main | | Recovery | | Animals | |
| Group | (mg/kg/day) | (mg/mL) | Male | Female | Male | Female | Male | Female |
| Control | 0 | 0 | 10 | 10 | 5 | 5 | 3 | 3 |
| Low Dose | 25 | 5 | 10 | 10 | — | — | 6 | 6 |
| Mid Dose | 50 | 10 | 10 | 10 | — | — | 6 | 6 |
| High Dose | 100 | 20 | 10 | 10 | 5 | 5 | 6 | 6 |

Male and female Sprague-Dawley rats, 10/sex/group, were administered 0, 25, 50 or 100 mg/kg noribogaine HCl daily by single oral gavage for 14 days. An additional 5 rats/sex/group in the 0 (control) and 100 mg/kg groups were retained for a 28 day recovery period during which no drug was administered. Six rats/sex/group (3 rats/sex controls) were similarly dosed and sampled on study days 1 and 14 for analysis of noribogaine-HCl concentrations in the blood. Rats were observed for mortality, clinical signs, body weight, food consumption, ophthalmology (pre-dose, during week 2, and at the end of recovery), hematology, coagulation, clinical chemistry, urinalysis, gross necropsy, organ weights and histopathology (full tissue panel, plus immunocytochemistry of 5 sections of the brain and spinal cord by staining for GFAP and Calbindin). There were no test article-related effects on mortality (none occurred), clinical signs, ophthalmoscopy, hematology, coagulation parameters, clinical chemistry, urinalysis, gross necropsy or histopathology. Food consumption and body weight were slightly reduced (food consumption: −4.7% in males and females; body weight: −5.5% in males and −2.6% in females) in the high dose (100 mg/kg) groups. Minor increases in liver weight in the mid- and high dose groups were not correlated with histopathologic changes and are considered incidental. No treatment-related differences in the brain were seen in sections stained for GFAP or Calbindin.

The NOAEL dose in this study was interpreted to be 100 mg/kg, the highest dose tested in the study.

### Example 7

### Fourteen Day Repeat Dose Toxicity and Toxicokinetics in Dogs

The objective of this study was to determine the toxicity profile of noribogaine HCl given following oral (gavage) administration to dogs for 14 days according to the following Table 3 below:

TABLE 3
Toxicity and Toxicokinetic Study in Dogs

| | | Dose Con- | Toxicology Animals | | | |
|---|---|---|---|---|---|---|
| Group | Dose Level | centration | Main | | Recovery | |
| Designation | (mg/kg/day) | (mg/mL) | Male | Female | Male | Female |
| Control | 0 | 0 | 4 | 4 | 4 | 4 |
| Low Dose | 0.5 | 0.1 | 4 | 4 | — | — |
| Mid Dose | 1.0 | 0.2 | 4 | 4 | — | — |
| High Dose | 5.0 | 1.0 | 4 | 4 | 4 | 4 |

Noribogaine HCl was administered to groups of 4 male and 4 female dogs by single oral gavage daily for 14 days at doses of 0, 0.5, 1.0 and 5.0 mg/kg/day. An additional group of 4 male and 4 female dogs received either the vehicle control or 5.0 mg/kg/day for 14 days and were held for an additional 28 days after cessation of dosing to assess recovery from any potential drug-induced changes. The study was conducted under GLP guidelines and included comprehensive examinations of clinical signs, body weight, clinical pathology parameters, ophthalmologic examinations, ECG recordings and analyses of plasma for bioanalytical measurement of drug levels at appropriate intervals during the study. At the termination of the dosing phase and at the termination of the recovery phase, all dogs were subjected to a complete post-mortem examination including gross examination of major organs and histologic examination of an extensive list of tissues. Additional sections of brain were obtained from cerebrum, cerebellus, brain stem and spinal cord and examined histologically to evaluate potential effects on brain histopathology. In addition, these sections were examined with immunohistochemical stains for GFAP for evidence of gliosis and Calbindin for a more comprehensive examination of cerebellar Purkinje cells. No evidence of adverse effect was observed in any dog from any treatment group during the dosing or recovery phase in clinical observations, body weights, clinical pathological parameters, ophthalmologic examinations, ECG recordings, or gross lesions at necropsy. The results of the plasma drug level measurements at Day 1 and Day 14 of the study are shown in the Table below. Noribogaine-HCl maximum plasma concentrations (Cₘₐₓ) were reached between 0.5 and 0.9 hours post-dosing, following which plasma concentrations gradually decreased over a period of up to 24 hours, except in the male dogs and female dogs of Group 4, for which significant levels of noribogaine were still detected at 24 h post-dosing on both Days 1 and 14.

The only target tissue identified in this study was the lacrimal gland of dogs receiving 5 mg/kg/day. The lacrimal gland changes were characterized by slight to moderate atrophy and degeneration of the acinar cells accompanied by slight to moderate accumulation of brown/yellow pigment and infiltration of mononuclear cells. There was an associated mononuclear infiltration in the draining mandibular lymph nodes of affected dogs in this dose group. Despite the appearance of isolated ocular abnormalities in several dogs in this high dose group on ophthalmologic examination, there was no clear association between these ocular signs and the appearance of the lacrimal gland changes suggesting that these morphologic changes did not result in sufficient functional abnormality of the gland to produce physical changes in exterior structures of the eye. There was no clear evidence of local irritation associated with drug treatment in these high dose dogs. No evidence of drug-induced effect was observed in any other tissue including the extensive sections of brain evaluated with conventional histopathology or with immunohistochemistry. Examination of the animals in the recovery group showed clear evidence of regeneration of this lacrimal gland change. While slight atrophy was still evident in the acinar cells of the gland after 28 days off drug, no evidence of continuing and ongoing degeneration or cellular infiltration was observed. The NOAEL in this study was 1 mg/kg/day based on the lacrimal gland changes at 5 mg/kg/day. The results are summarized in Tables 4 and 5.

TABLE 4
Mean plasma toxicokinetic parameters for
noribogaine in male dogs on days 1 and 14

| | Gr 2 - 0.5 mg/kg | | Gr 3 - 1.0 mg/kg | | Gr 4 - 5.0 mg/kg | |
|---|---|---|---|---|---|---|
| Parameters | D 1 | D 14 | D 1 | D 14 | D 1 | D 14 |
| T_{1/2} (h) | 1.3 | 1.3 | 1.2 | 1.8 | 4.7 | 6.5 |
| Tₘₐₓ (h) | 0.7 | 0.7 | 0.9 | 0.8 | 0.6 | 0.9 |
| Cₘₐₓ (ng/ml) | 28.8 | 29.4 | 58.6 | 67.6 | 693 | 716 |
| AUC₀₋ₗₐₛₜ | 46.6 | 53.2 | 102.5 | 172.3 | 3515.0 | 6403.3 |
| (hr*ng/ml) | | | | | | |
| AUC_{0-24 h} | 59.7 | 64.5 | 119.8 | 210.4 | 3515.0 | 6403.3 |
| (hr*ng/ml) | | | | | | |
| AUC_{0-∞} | 67.8 | 68.2 | 120.8 | 195.7 | 3630.5 | 6961.4 |
| (hr*ng/ml) | | | | | | |

TABLE 5
Mean plasma toxicokinetic parameters for noribogaine
in female dogs on days 1 and 14

| | Gr 2 - 0.5 mg/kg | | Gr 3 - 1.0 mg/kg | | Gr 4 - 5.0 mg/kg | |
|---|---|---|---|---|---|---|
| Parameters | D 1 | D 14 | D 1 | D 14 | D 1 | D 14 |
| T_{1/2} (h) | 1.0 | 1.1 | 1.4 | 1.6 | 4.3 | 5.7 |
| Tₘₐₓ (h) | 0.5 | 1.0 | 0.8 | 0.5 | 0.6 | 0.6 |
| Cₘₐₓ (ng/ml) | 25.3 | 29.8 | 68.5 | 74.1 | 691 | 683 |
| AUC₀₋ₗₐₛₜ | 31.5 | 35.4 | 148.9 | 169.0 | 3367.9 | 5951.2 |
| (hr*ng/ml) | | | | | | |
| AUC_{0-24 h} | 40.4 | 55.0 | 176.2 | 203.7 | 3367.9 | 5951.2 |
| (hr*ng/ml) | | | | | | |
| AUC_{0-∞} | 44.9 | 45.7 | 165.3 | 197.0 | 3425.7 | 6283.2 |
| (hr*ng/ml) | | | | | | |

### Example 8

### Human Pharmacokinetic Studies

In double blind studies, fasting healthy volunteers (6 per cohort) were treated once orally with a tablet of noribogaine HCl. In escalating cohorts, the volunteers received 3 mg, 10 mg, 30 mg or 60 mg noribogaine. The results are provided below. All parameters were linear and no clinically relevant adverse effects were observed in the trial.

The subject mean serum levels over time of noribogaine free base from a single dose of 3 mg noribogaine free base under fasting conditions were plotted. The mean Cₘₐₓ of 5.2 ng/ml was observed 1.9 hours after administration, while the mean AUC/24 hr of 3.1 ng/ml was obtained.

The subject mean serum levels over time of noribogaine free base from a single dose of 10 mg noribogaine free base under fasting conditions were plotted. The mean Cₘₐₓ of 14.5 ng/ml was observed 2.9 hours after administration, while the mean AUC/24 hr of 10.6 ng/ml was obtained.

The subject mean serum levels over time of noribogaine free base from a single dose of 30 mg noribogaine free base under fasting conditions were plotted. The mean Cₘₐₓ of 55.9 ng/ml was observed between 1.75 hours after administration, while the mean AUC/24 of 29.2 ng/ml was obtained.

The subject mean serum levels over time of noribogaine free base from a single dose of 60 mg noribogaine free base under fasting conditions were plotted. The mean Cₘₐₓ of 116 ng/ml was observed between 1.75 hours after administration, while the mean AUC/24 ng/ml of 61 was obtained.

The subject mean serum levels over time of noribogaine free base for all 4 cohorts were plotted. The extrapolated dosage of noribogaine free base required to provide a Cₘₐₓ ranging from about 5.2 ng/ml to about 1980 ng/ml and an AUC/24 hr of about 3.1 ng/ml to about 1100 ng/ml was determined.

### Example 9

### Pharmacokinetics and Pharmacodynamics of Noribogaine in Humans

Thirty-six healthy, drug-free male volunteers, aged between 18-55 years, were enrolled in and completed the study. This was an ascending single-dose, placebo-controlled, randomized double blind, parallel group study. Mean (SD) age was 22.0 (3.3) years, mean (SD) height was 1.82 (0.08) m, and mean (SD) weight was 78.0 (9.2) kg. Twenty-six subjects were Caucasian, 3 were Asian, 1 Maori, 1 Pacific Islander, and 5 Other. The protocol for this study was approved by the Lower South Regional Ethics Committee (LRS/Dec. 6, 2015), and the study was registered with the Australian New Zealand Clinical Trial Registry (ACTRN12612000821897). All subjects provided signed informed consent prior to enrolment, and were assessed as suitable to participate based on review of medical history, physical examination, safety laboratory tests, vital signs and ECG.

Within each dose level, 6 participants were randomized to receive noribogaine and 3 to receive placebo, based on a computer-generated random code. Dosing began with the lowest noribogaine dose, and subsequent cohorts received the next highest dose after the safety, tolerability, and blinded pharmacokinetics of the completed cohort were reviewed and dose-escalation approved by an independent Data Safety Monitoring Board. Blinded study drug was administered as a capsule with 240 ml of water after an overnight fast of at least 10 hours. Participants did not receive any food until at least 5 hours post-dose. Participants were confined to the study site from 12 hours prior to drug administration, until 72 hours post-dose, and there were subsequent outpatient assessments until 216 hours post-dose.

Blood was obtained for pharmacokinetic assessments pre-dose and then at 0.25, 0.5, 0.75, 1.0, 1.25, 1.5, 2, 2.5, 3, 3.5, 4, 4.5, 5, 5.5, 6, 7, 8, 10, 12, 14, 18, 24, 30, 36, 48, 60, 72, 96, 120, 168 and 216 hours post-dose. Samples were centrifuged and plasma stored at −70° C. until analyzed. Block 24 hour urine collections were obtained following study drug administration for the 30 and 60 mg cohorts. Aliquots were frozen at −20° C. until analyzed.

Pulse oximetry and capnography data were collected continuously using a GE Carescape B650 monitoring system from 2 hours prior to dosing and until six hours after dosing, and thereafter at 12, 24, 48 and 72 hours post-dosing. Additional oximetry data were collected at 120, 168 and 216 hours. Pupillary miosis was assessed by pupillometry. Dark-adapted pupil diameter was measured in triplicate using a Neuroptics PLR-200 pupillometer under standardized light intensity (<5 lux) pre-dose, and at 2, 4, 6, 12, 24, 48, 72, 96, 120, 168 and 216 hours post-dosing.

Plasma noribogaine concentrations were determined in the 3 mg and 10 mg dose groups using a validated, sensitive LCMSMS method. Sample preparation involved double extraction of basified plasma samples with tert-butyl methyl ether, drying the samples under a stream of nitrogen and reconstitution of sample with acetonitrile:B.P. water (5:95, v/v) containing 0.1% (v/v) formic acid. The compounds were separated by a 150×2.0 mm Luna 5 μm C18 column and detected with a triple-quadruple API 4000 or 5000 mass spectrometer using electrospray ionization in positive mode and multiple reaction monitoring. Noribogaine-d₄ was used as the internal standard. The precursor-product ion transition values for noribogaine were m/z 297.6->122.3, and for the internal standard noribogaine-d₄ m/z 301.1->122.2. Analyst® software was used for data acquisition and processing. The ratio of the peak area of noribogaine to the internal standard noribogaine-d₄ was used for calibration and measurement of the unknown concentration of noribogaine. The lower limit of quantification (LLOQ) was 0.025 ng/ml noribogaine. The calibration curve was between 0.025 and 25.600 ng/ml noribogaine. Mobile phase A was acetonitrile:B.P. water (5:95, v/v) containing 0.1% (v/v) formic acid, and mobile phase B was acetonitrile:B.P. water (95:5, v/v) containing 0.1% (v/v) formic acid. Total run time was 6 minutes. Binary flow: Initial concentration was 8% mobile phase B; hold at 8% mobile phase B for 0.5 minutes and linear rise to 90% mobile phase B over 1.5 minutes; hold at 90% mobile phase B for 1 minute and then drop back to 8% mobile phase B over 0.01 minute. Equilibrate system for 3 minutes. Total run time was 6 minutes. Within- and between-day assay precision was <9%, and within- and between-day assay accuracy was <9%.

Plasma noribogaine concentrations were determined in the 30 mg and 60 mg dose groups using a validated, sensitive LCMSMS method. Sample preparation involved deproteinization of plasma samples with acetonitrile and dilution of sample with 0.1% (v/v) formic acid. The compounds were separated by a 150×2.0 mm Luna 5 m C18 column and detected with a triple-quadruple API 4000 or 5000 mass spectrometer using electrospray ionization in positive mode and multiple reaction monitoring. Noribogaine-d₄ was used as the internal standard. The precursor-product ion transition values for noribogaine were m/z 297.6->122.3, and for the internal standard noribogaine-d₄ m/z 301.1->122.2. Analyst® software was used for data acquisition and processing. The ratio of the peak area of noribogaine to the internal standard noribogaine-d₄ was used for calibration and measurement of the unknown concentration of noribogaine. The LLOQ was 0.50 ng/ml noribogaine. The calibration curve was between 0.50 and 256.00 ng/ml noribogaine. Mobile phase was the same as method A, and binary flow was also the same as method A. The within- and between-day assay precision was <9%, and the within- and between-day assay accuracy was <9%.

Plasma noribogaine glucuronide concentrations were determined in the 30 mg and 60 mg dose groups using a validated sensitive LCMSMS method. Sample preparation involved deproteinization of plasma samples with acetonitrile, drying the samples under a stream of nitrogen and reconstitution of sample with acetonitrile:B.P. water (5:95, v/v) containing 0.1% (v/v) formic acid. The compounds were separated by a 150×2.0 mm Luna 5 m C18 column and detected with a triple-quadruple API 4000 or 5000 mass spectrometer using electrospray ionization in positive mode and multiple reaction monitoring. Noribogaine-d₄ was used as the internal standard. The precursor-product ion transition values for noribogaine glucuronide were m/z 472.8->297.3, and for the internal standard noribogaine-d₄ m/z 301.1->122.2. Analyst® software was used for data acquisition and processing. The ratio of the peak area of noribogaine glucuronide to the internal standard noribogaine-d₄ was used for calibration and measurement of the unknown concentration of noribogaine glucuronide. The LLOQ was 0.050 ng/ml noribogaine glucuronide. The calibration curve was between 0.050 and 6.400 ng/ml noribogaine glucuronide. Mobile phases was the same as method A. Binary flow: Initial concentration was 6% mobile phase B; hold at 6% mobile phase B for 0.5 minutes and linear rise to 90% mobile phase B over 2 minutes; hold at 90% mobile phase B for 1 minute and then drop back to 6% mobile phase B over 0.01 minute. Equilibrate system for 3.5 minutes. Total run time was 7 minutes. The within- and between-day assay precision was <11%, and the within- and between-day assay accuracy was <10%.

Urine noribogaine and noribogaine glucuronide concentrations were determined in the 30 mg and 60 mg dose groups using a validated sensitive LCMSMS method. Sample preparation involved deproteinization of urine samples with acetonitrile and dilution of the sample with 0.1% (v/v) formic acid. The compounds were separated by a 150×2.0 mm Luna 5 m C18 column and detected with a triple-quadruple API 4000 or 5000 mass spectrometer using electrospray ionization in positive mode and multiple reaction monitoring. Noribogaine-d₄ was used as the internal standard. The precursor-product ion transition values for noribogaine were m/z 297.6->122.3, noribogaine glucuronide m/z 472.8->297.3, and for the internal standard noribogaine-d₄ m/z 301.1->122.2. Analyst® software was used for data acquisition and processing. The ratios of the peak area of noribogaine and noribogaine glucuronide to the internal standard noribogaine-d₄ were used for calibration and measurement of the unknown concentration of noribogaine and its glucuronide. Assay LLOQ was 20.0 ng/ml for noribogaine and 2.0 ng/ml for noribogaine glucuronide. The calibration curve was between 20.0 and 5120.0 ng/ml noribogaine, and 2.0 and 512.0 ng/ml noribogaine glucuronide. Mobile phases were as described in method A, and binary flow as in method C. The within- and between-day assay precision was <13%, and within- and between-day assay accuracy was <12%.

Noribogaine and noribogaine glucuronide concentrations above the limit of quantification were used to calculate pharmacokinetic parameters using model-independent methods. The maximum plasma concentration (Cmax) and time to maximum plasma concentration (Tmax) were the observed values. Plasma concentration data in the post-distribution phase of the plasma concentration-time plot were fitted using linear regression to the formula ln C=ln Co×t.Kel, where Co was the zero-time intercept of the extrapolated terminal phase and Kel was the terminal elimination rate constant. The half-life (t_{1/2}) was determined using the formula t_{1/2}=0.693/Kel. The area under the concentration-time curve (AUC) from time zero to the last determined concentration-time point (tf) in the post distribution phase was calculated using the trapezoidal rule. The area under the curve from the last concentration-time point in the post distribution phase (Ctf) to time infinity was calculated from AUC_{t-∞}=Ctf/Kel. The concentration used for Ctf was the last determined value above the LLOQ at the time point. The total AUC_{0-∞} was obtained by adding AUC_{tf} and AUC_{t-∞}. Noribogaine apparent clearance (CL/F) was determined using the formula CL/F=Dose/AUC_{0-∞}×1000, and apparent volume of distribution (Vd/F) was determined using the formula Vd/F=(CL/F)/Kel. Total urine noribogaine was the sum of both analytes.

Summary statistics (means, standard deviations, and coefficients of variation) were determined for each dose group for safety laboratory test data, ECG and pharmacokinetic parameters, and pharmacodynamic variables. Categorical variables were analysed using counts and percentages. Dose-proportionality of AUC and Cmax was assessed using linear regression. The effect of dose on pharmacodynamic parameter values over time was assessed using two-factor analysis of variance (ANOVA). Pairwise comparisons (with Tukey-Kramer adjustment) between each dose group to the placebo were conducted at each time point using the least squares estimates obtained from the ANOVA, using SAS Proc Mixed (SAS ver 6.0).

### Results

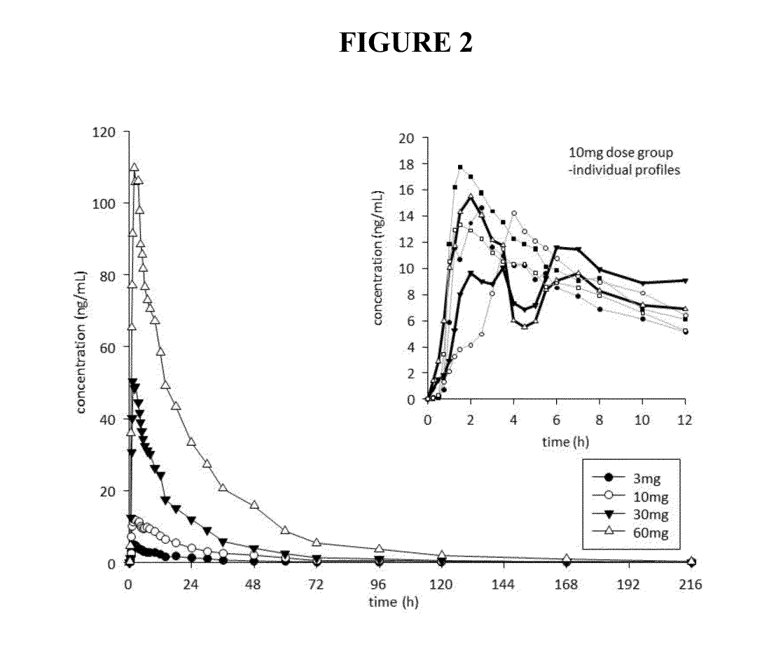

Pharmacokinetics: Mean plasma concentration-time plots of noribogaine are shown in FIG. 2, and mean pharmacokinetic parameters are shown in Table 6.

TABLE 6

| | | | |
|---|---|---|---|
| 3 mg (n = 6) | 10 mg (n = 6) | 30 mg (n = 6) | 60 mg (n = 6) |
| (mean (SD)) | (mean (SD)) | (mean (SD)) | (mean (SD) |

Noribogaine

| | | | | |
|---|---|---|---|---|
| AUC_{0-∞} | 74.2 (13.1) | 254.5 (78.9) | 700.4 (223.3) | 1962.2 (726.5) |
| (ng · hr/ml) | | | | |
| AUC₀₋₂₁₆ | 72.2 (13.2) | 251.4 (78.5) | 677.6 (221.1) | 1935.4 (725.4) |
| (ng · hr/ml) | | | | |
| Cmax | 5.2 (1.4) | 14.5 (2.1) | 55.9 (14.8) | 116.0 (22.5) |
| (ng/ml) | | | | |
| Tmax (hr) | 1.9 (0.6) | 2.9 (1.8) | 1.8 (0.6) | 2.4 (0.6) |
| t_{1/2} (hr) | 40.9 (8.7) | 49.2 (11.5) | 27.6 (7.0)) | 29.1 (9.3) |
| Vd/F (L) | 2485.1 (801.5) | 3085.8 (1197.0) | 1850.8 (707.9) | 1416.8 (670.1) |
| CL/F (L/h) | 41.4 (7.0) | 42.3 (12.0) | 46.9 (16.4) | 34.0 (11.4) |

Noribogaine glucuronide

| | | | | |
|---|---|---|---|---|
| AUC_{0-∞} | — | — | 25.8 (9.3) | 67.1 (21.9) |
| (ng · hr/ml) | | | | |
| AUC₀₋₂₁₆ | — | — | 25.7 (9.1) | 65.0 (21.5) |
| (ng · hr/ml) | | | | |
| Cmax | — | — | 1.8 (0.6) | 4.1 (1.2) |
| (ng/ml) | | | | |
| Tmax (hr) | — | — | 3.0 (0.6) | 3.8 (1.2) |
| t_{1/2} (hr) | — | — | 20.6 (4.9) | 23.1 (3.0) |

Noribogaine was rapidly absorbed, with peak concentrations occurring 2-3 hours after oral dosing. Fluctuations in individual distribution-phase concentration-time profiles may suggest the possibility of enterohepatic recirculation (see highlighted individual 4-8 hour profiles in FIG. 2, insert). Both Cmax and AUC increased linearly with dose (Table 6, upper panel). Mean half-life estimates of 28-50 hours were observed across dose groups for noribogaine. Volume of distribution was extensive (1417-3086 L across dose groups).

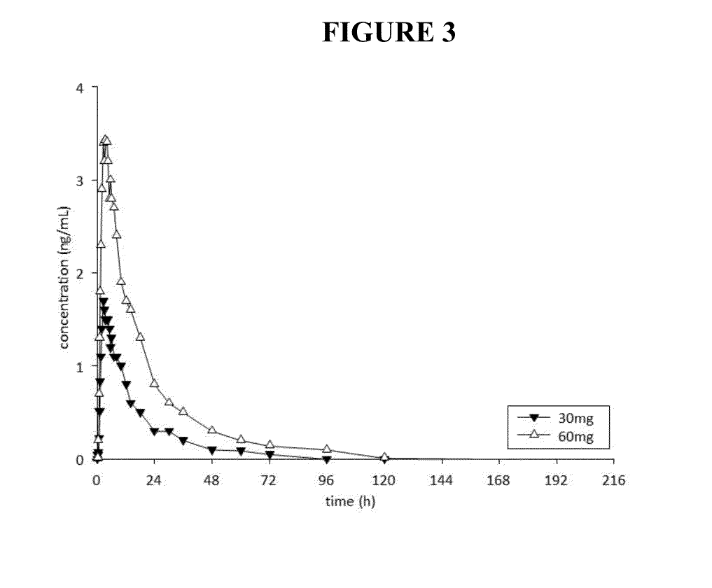

Mean plasma noribogaine glucuronide concentration-time plots for the 30 mg and 60 mg dose group are shown in FIG. 3, and mean pharmacokinetic parameters are shown in Table 6, lower panel. Noribogaine glucuronide was detected in all subjects by 0.75 hours, with peak concentrations occurring 3-4 hours after noribogaine dosing. Mean half-life of 21-23 hours was estimated for plasma noribogaine glucuronide. The proportion of noribogaine glucuronide Cmax and AUC relative to noribogaine was 3-4% for both dose groups. Total urine noribogaine elimination was 1.16 mg and 0.82 mg for the 30 mg and 60 mg dose groups respectively, representing 3.9% and 1.4% of the doses administered.

Pharmacodynamics: There was no evidence of pupillary constriction in subjects dosed with noribogaine. No between-dose group differences in pupil diameter were detected over time. After adjusting for baseline differences, comparison of each dose group with placebo by ANOVA showed no statistically significant differences (p>0.9).

Noribogaine treatment showed no analgesic effect in the cold pressor test. Analgesic effect was assessed based on duration of hand immersion in ice water and on visual analog scale (VAS) pain scores upon hand removal from the water bath. For duration of hand immersion, after adjusting for baseline differences, comparison of each dose group with placebo by ANOVA showed no statistically significant differences (p>0.9). Similarly, for VAS pain scores, after adjusting for baseline differences, comparison of each dose group with placebo by ANOVA showed no statistically significant differences (p=0.17).

### Example 10

### Safety and Tolerability of Noribogaine in Healthy Humans

Safety and tolerability of noribogaine were tested in the group of volunteers from Example 9. Cold pressor testing was conducted in 1° C. water according to the method of Mitchell et al. (J Pain 5:233-237, 2004) pre-dose, 6, 24, 48, 72 and 216 hours post-dosing. Safety evaluations included clinical monitoring, recording of adverse events (AEs), safety laboratory tests, vital signs, ECG telemetry from −2 h to 6 h after dosing, and 12-lead electrocardiograms (ECGs) up to 216 hours post-dosing.

### Results

A total of thirteen adverse events were reported by seven participants (Table 7). Six adverse events were reported by three participants in the placebo group, five adverse events were reported by two subjects in the 3 mg dose group, and one adverse event was reported by single subjects in the 10 mg and 30 mg dose groups, respectively. The most common adverse events were headache (four reports) and epistaxis (two reports). All adverse events were of mild-moderate intensity, and all resolved prior to study completion. There were no changes in vital signs or safety laboratory tests of note. In particular, there were no changes in oximetry or capnography, or changes in respiratory rate. There were no QTcF values >500 msec at any time. One subject dosed with 10 mg noribogaine had a single increase in QTcF of >60 msec at 24 hours post-dosing.

TABLE 7

| Dose (mg) | Mild | Moderate | Severe |
|---|---|---|---|
| Placebo | Blepharitis | Epistaxis | — |
| | Bruising | | |
| | Dry Skin | | |
| | Eye pain, nonspecific | | |
| | Infection at cannula site | | |
| 3 | Back pain | Headache | — |
| | Dizziness | | |
| | Epistaxis | | |
| | Headache | | |
| 10 | Headache | — | — |
| 30 | Headache | — | — |
| 60 | — | — | — |

### Example 11

### Therapeutic Window for Treatment with Noribogaine in Humans

This example is to illustrate that noribogaine can be administered at a therapeutic dosing while maintaining an acceptable QT interval. While the therapy employed is directed to opioid-dependent participants in a randomized, placebo-controlled, double-blind trial, the results show that a therapeutic window can be established for noribogaine.

The specifics of this example include patients who had been receiving methadone treatment as the opioid substitution therapy, but were transferred to morphine treatment prior to noribogaine administration. This was done to avoid negative noribogaine-methadone interactions that are not observed between noribogaine and methadone. See PCT Application No. PCT/US2013/069235, filed Nov. 8, 2013, which is incorporated herein by reference in its entirety.

Three cohorts of nine (9) subjects (6 administered noribogaine and 3 administered placebo in each cohort) were evaluated for tolerability, pharmacokinetics, and efficacy. Cohort 1 received a single dose of 60 mg noribogaine or placebo. Cohort 2 received a single dose of 120 mg noribogaine or placebo. Cohort 3 received a single dose of 180 mg noribogaine or placebo. Treatment was administered 2 hours after last morphine dose and the time to resumption of morphine (opioid substitution treatment, OST) was determined. Few adverse effects of noribogaine were observed in any of the participants, including no hallucinatory effects. Table 8 shows the reported adverse events for each treatment.

TABLE 8
Treatment Emergent Adverse Events Summary

| System Organ Class | | Placebo | 60 mg | 120 mg | 180 mg |
|---|---|---|---|---|---|
| Preferred Term | | (N = 9) | (N = 6) | (N = 6) | (N = 6) |
| Number of Subjects Reporting | | 19:7 (77.8%) | 15:5 (83.3%) | 28:6 (100.0%) | 17:4 (66.7%) |
| any AEs | | | | | |
| Ear and Labyrinth Disorders | | 0 | 0 | 2:2 (33.3%) | 0 |
| | Tinnitus | 0 | 0 | 2:2 (33.3%) | 0 |
| Eye Disorders | | 2:2 (22.2%) | 3:3 (50.0%) | 5:5 (83.3%) | 5:4 (66.7%) |
| | Visual Impairment | 2:2 (22.2%) | 2:2 (33.3%) | 5:5 (83.3%) | 5:4 (66.7%) |
| | Dry Eye | 0 | 1:1 (16.7%) | 0 | 0 |
| Gastrointestinal Disorders | | 3:2 (22.2%) | 2:2 (33.3%) | 7:2 (33.3%) | 4:2 (33.3%) |
| | Nausea | 1:1 (11.1%) | 0 | 3:2 (33.3%) | 2:2 (33.3%) |
| | Dry Mouth | 0 | 0 | 1:1 (16.7%) | 1:1 (16.7%) |
| | Vomiting | 0 | 0 | 2:1 (16.7%) | 1:1 (16.7%) |
| | Diarrhoea | 1:1 (11.1%) | 0 | 1:1 (16.7%) | 0 |
| | Dyspepsia | 1:1 (11.1%) | 2:2 (33.3%) | 0 | 0 |
| General Disorders and Administration | | 4:3 (33.3%) | 0 | 2:2 (33.3%) | 1:1 (16.7%) |
| Site Conditions | | | | | |
| | Catheter Site Related Reaction | 0 | 0 | 0 | 1:1 (16.7%) |
| | Catheter Site Pain | 3:2 (22.2%) | 0 | 2:2 (33.3%) | 0 |
| | Malaise | 1:1 (11.1%) | 0 | 0 | 0 |
| Infections and Infestations | | 1:1 (11.1%) | 0 | 1:1 (16.7%) | 2:2 (33.3%) |
| | Cellulitis | 0 | 0 | 1:1 (16.7%) | 1:1 (16.7%) |
| | Urinary Tract Infection | 0 | 0 | 0 | 1:1 (16.7%) |
| | Catheter Site Infection | 1:1 (11.1%) | 0 | 0 | 0 |
| Musculoskeletal and Connective | | 1:1 (11.1%) | 2:1 (16.7%) | 0 | 2:2 (33.3%) |
| Tissue Disorders | | | | | |
| | Back Pain | 1:1 (11.1%) | 2:1 (16.7%) | 0 | 1:1 (16.7%) |
| | Limb Discomfort | 0 | 0 | 0 | 1:1 (16.7%) |
| Nervous System Disorders | | 7:5 (55.6%) | 7:4 (66.7%) | 5:4 (66.7%) | 3:2 (33.3%) |
| | Headache | 6:5 (55.6%) | 7:4 (66.7%) | 2:2 (33.3%) | 3:2 (33.3%) |
| | Hyperaesthesia | 0 | 0 | 1:1 (16.7%) | 0 |
| | Pseudoparalysis | 0 | 0 | 1:1 (16.7%) | 0 |
| | Tremor | 0 | 0 | 1:1 (16.7%) | 0 |
| | Somnoience | 1:1 (11.1%) | 0 | 0 | 0 |
| Psychiatric Disorders | | 1:1 (11.1%) | 1:1 (16.7%) | 0 | 0 |
| | Depressed Mood | 0 | 1:1 (16.7%) | 0 | 0 |
| | Euphoric Mood | 1:1 (11.1%) | 0 | 0 | 0 |
| Respiratory, Thoracic and | | 0 | 0 | 4:2 (33.3%) | 0 |
| Mediastinal Disorders | | | | | |
| | Epistaxis | 0 | 0 | 2:1 (16.7%) | 0 |
| | Oropharyngeal Pain | 0 | 0 | 1:1 (16.7%) | 0 |
| | Rhinorrhoea | 0 | 0 | 1:1 (16.7%) | 0 |
| Skin and Subcutaneous | | 0 | 0 | 2:1 (16.7%) | 0 |
| Tissue Disorders | | | | | |
| | Skin Discomfort | 0 | 0 | 1:1 (16.7%) | 0 |
| | Skin Irritation | 0 | 0 | 1:1 (16.7%) | 0 |

Note:
Within each system organ class, Preferred Terms are presented by descending incidence of descending dosages groups and then the placebo group.
Note:
N = number of subjects in the safety population.

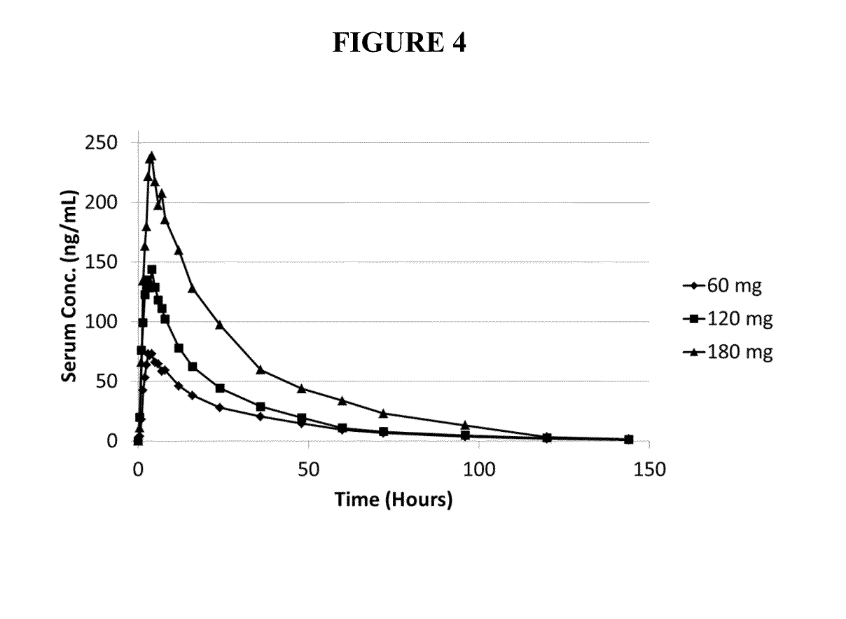

FIG. 4 indicates the serum noribogaine concentration over time for each cohort. Data is provided in Table 9.

TABLE 9
Serum noribogaine concentration over time for each cohort.

| | 0 | 0.5 | 1 | 1.5 | 2 | 2.5 | 3 | 3.5 | 4 | 5 | 6 | 7 |
|---|---|---|---|---|---|---|---|---|---|---|---|---|
| 60 mg | 0 | 3.75 | 17.86 | 42.54 | 53.01 | 63.71 | 72.95 | 71.43 | 72.89 | 65.74 | 64.47 | 58.26 |
| 120 mg | 0 | 19.66 | 76.04 | 98.80 | 122.67 | 135.05 | 130.55 | 128.20 | 143.90 | 128.76 | 118.16 | 110.76 |
| 180 mg | 0 | 10.78 | 65.68 | 134.19 | 163.32 | 179.63 | 221.67 | 236.27 | 238.87 | 217.26 | 197.10 | 207.47 |

| | 8 | 12 | 16 | 24 | 36 | 48 | 60 | 72 | 96 | 120 | 144 |
|---|---|---|---|---|---|---|---|---|---|---|---|
| 60 mg | 59.01 | 46.10 | 37.99 | 27.76 | 20.32 | 14.56 | 9.32 | 6.55 | 3.41 | 1.64 | 0.89 |
| 120 mg | 102.12 | 77.88 | 62.24 | 44.19 | 28.83 | 19.54 | 10.75 | 7.75 | 4.66 | 2.45 | 1.20 |
| 180 mg | 185.55 | 159.53 | 127.63 | 97.34 | 59.50 | 43.75 | 33.69 | 23.06 | 13.04 | 3.37 | 1.61 |

### Results

Pharmacokinetic results for each cohort are given in Table 10. Maximum serum concentration of noribogaine (Cmax) increased in a dose-dependent manner. Time to Cmax (Tmax) was similar in all three cohorts. Mean half-life of serum noribogaine was similar to that observed in healthy patients.

TABLE 10
Pharmacokinetic results from the Patients in Phase IB Study

| | Cohort 1 (60 mg) | Cohort 2 (120 mg) | Cohort 3 (180 mg) |
|---|---|---|---|
| | Data (mean ± SD) | Data (mean ± SD) | Data (mean ± SD) |
| PK parameter | [range] | [range] | [range] |
| Cmax | 81.64 ± 23.77 | 172.79 ± 30.73 | 267.88 ± 46.92 |
| (ng/ml) | [41.29-113.21] | [138.84-229.55] | [204.85-338.21] |
| Tmax | 3.59 ± 0.92 | 2.99 ± 1.23 | 4.41 ± 1.80 |
| (hours) | [2.50-5.00] | [0.98-4.02] | [3.00-8.00] |
| AUC_{(0-T)} | 2018.01 ± 613.91 | 3226.38 ± 1544.26 | 6523.28 ± 2909.80 |
| (ng · hr/ml) | [1094.46-2533.44] | [1559.37-5638.98] | [3716.69-10353.12] |
| AUC_{(0-¥)} | 2060.31 ± 609.39 | 3280.50 ± 1581.43 | 6887.67 ± 3488.91 |
| (ng · hr/ml) | [1122.29-2551.63] | [1595.84-5768.52] | [3734.21-12280.91] |
| Half-life | 29.32 ± 7.28 | 30.45 ± 9.14 | 23.94 ± 5.54 |
| (hrs) | [18.26-37.33] | [21.85-48.33] | [19.32-34.90] |
| Vd/F | 1440.7 ± 854.0 | 2106.43 ± 1644.54 | 1032.19 ± 365.30 |
| | [619.5-2772.5] | [824.24-5243.78] | [581.18-1608.98] |
| Cl/F | 32.14 ± 12.38 | 44.68 ± 21.40 | 31.47 ± 13.12 |
| | [23.51-53.46] | [20.80-75.20] | [14.66-48.20] |

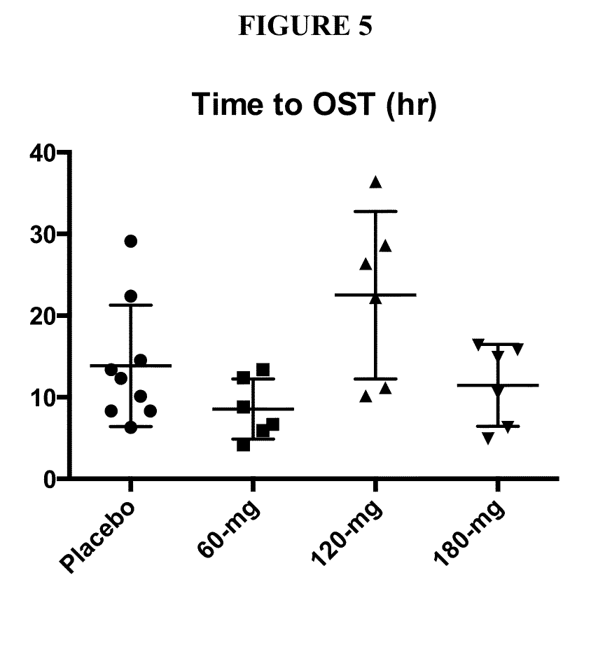

FIG. 5 indicates the time to resumption of morphine (OST) for patients treated with placebo (circles), 60 mg noribogaine (squares), 120 mg noribogaine (triangles), and 180 mg noribogaine (inverted triangles). Patients receiving a single 120 mg dose of noribogaine exhibited an average time to resumption of opioids of greater than 20 hours. Patients receiving a single 180 mg dose of noribogaine exhibited an average time to resumption of opioids similar to that of placebo. This demonstrates that increasing the dose of noribogaine to 180 mg results in a shorter time to resumption of OST than observed in patients receiving 120 mg noribogaine. Time to resumption of OST after treatment with 180 mg was still longer than untreated patients (7 hours, not shown) or those administered 60 mg noribogaine.

Patients were evaluated based on the Clinical Opiate Withdrawal Scale (COWS), Subjective Opiate Withdrawal Scale (SOWS), and Objective Opiate Withdrawal Scale (OOWS) scoring systems over the period of time between administration of noribogaine (or placebo) until resumption of OST. These scales are outlined in Guidelines for the Psychosocially Assisted Pharmacological Treatment of Opioid Dependence, World Health Organization, Geneva (2009), Annex 10, which is incorporated herein by reference in its entirety. The scales measure the intensity of withdrawal symptoms, based on clinical, subjective, and objective indicia.

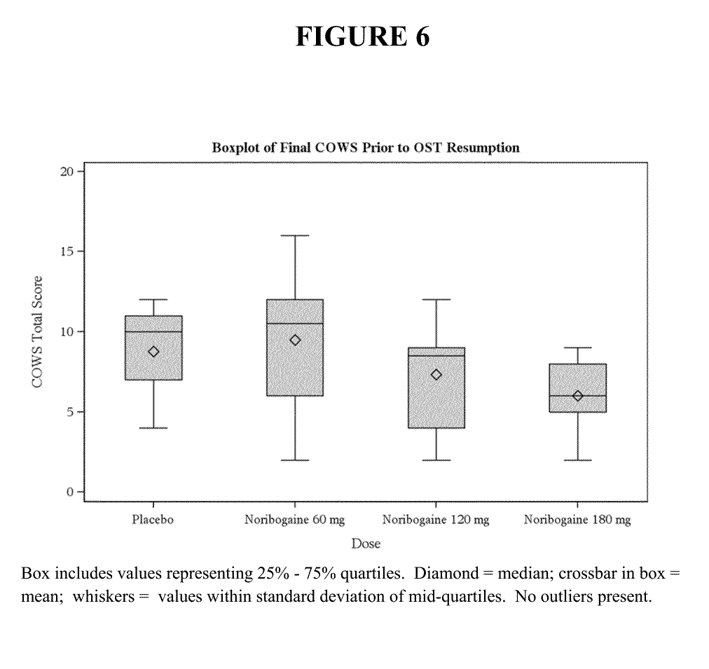

FIG. 6 shows the COWS scores at time of resumption of OST for each cohort. Box includes values representing 25%-75% quartiles. Diamond=median; crossbar in box=mean; whiskers=values within standard deviation of mid-quartiles. No outliers present. The highly variable COWS scores across and within each cohort indicates that patients were resuming opiates without relation to the intensity of withdrawal. This was also reflected in SOWS and OOWS scores at the time of resumption of OST.

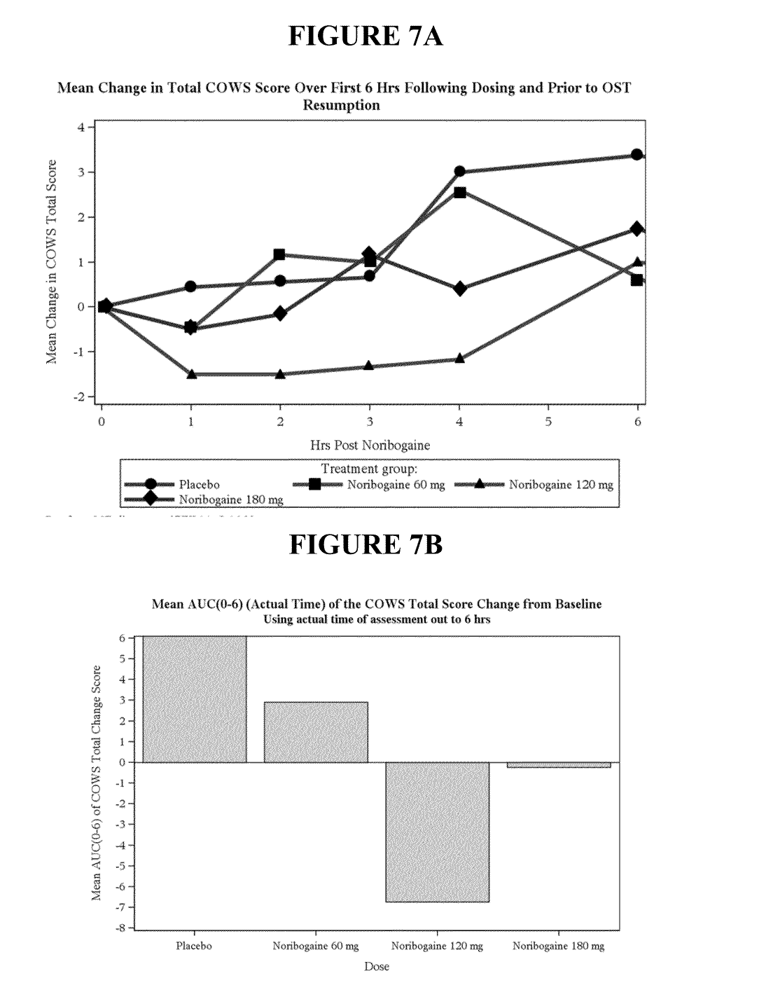

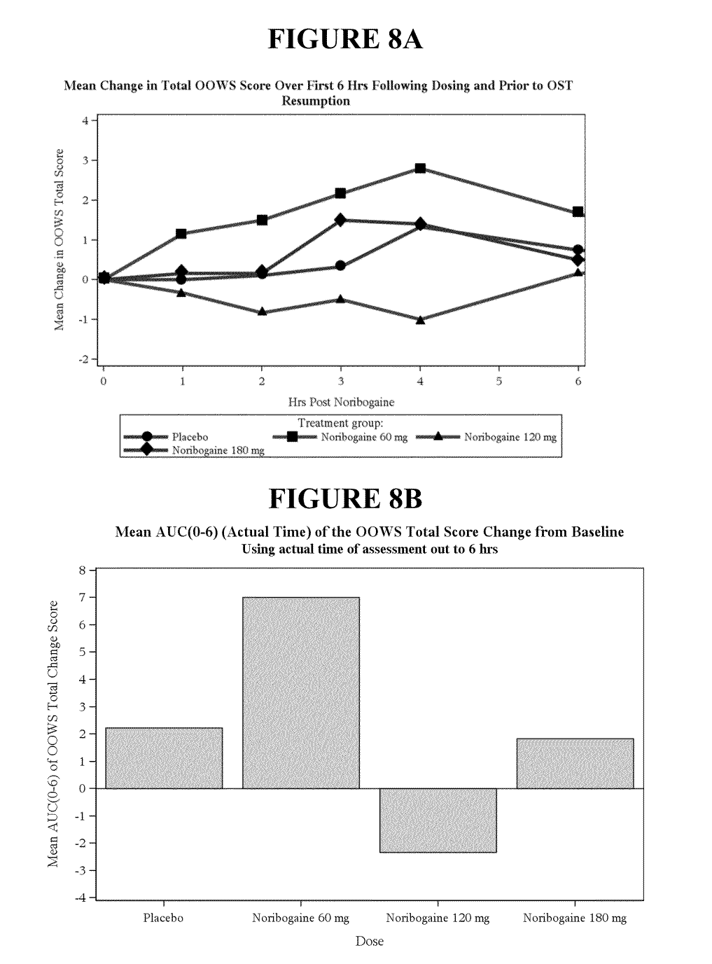

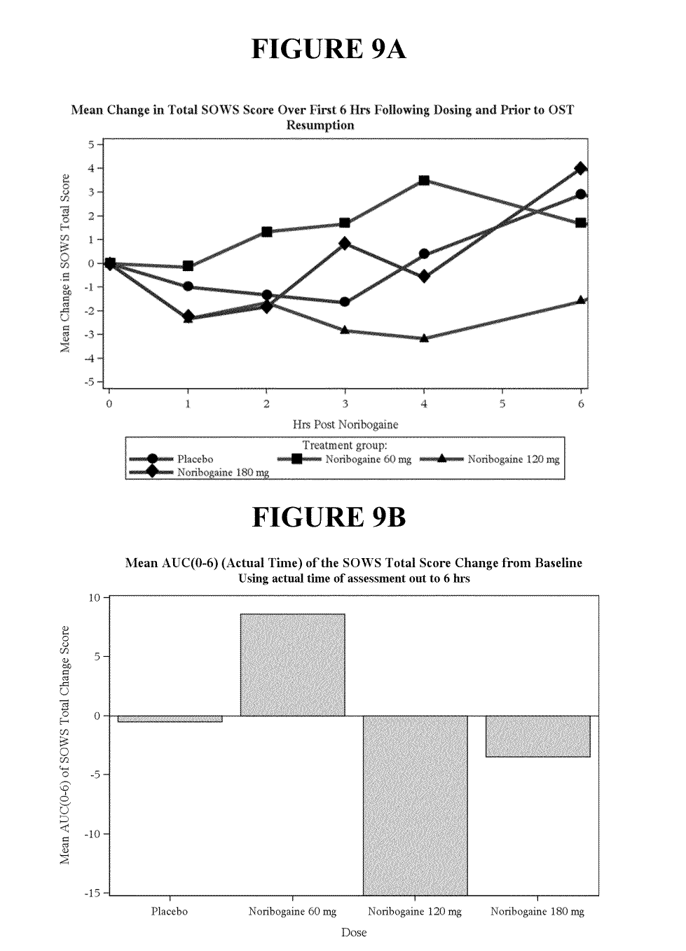

FIG. 7A shows the mean change in total COWS scores over the first six hours following dosing and prior to resumption of OST. FIG. 7B shows the mean AUC(0-6 hours) of the COWS total score change from baseline. FIG. 8A shows the mean change in total OOWS scores over the first six hours following dosing and prior to resumption of OST. FIG. 8B shows the mean AUC(0-6 hours) of the OOWS total score change from baseline. FIG. 9A shows the mean change in total SOWS scores over the first six hours following dosing and prior to resumption of OST. FIG. 9B shows the mean AUC(0-6 hours) of the SOWS total score change from baseline. These data indicate that withdrawal symptoms get worse over time after cessation of OST, and that patients administered placebo experience generally worse withdrawal symptoms over that period. Patients who received 120 mg noribogaine generally experienced fewer withdrawal symptoms than the other patients, regardless of the scale used. Patients administered placebo generally experienced more withdrawal symptoms than patients who were administered noribogaine.

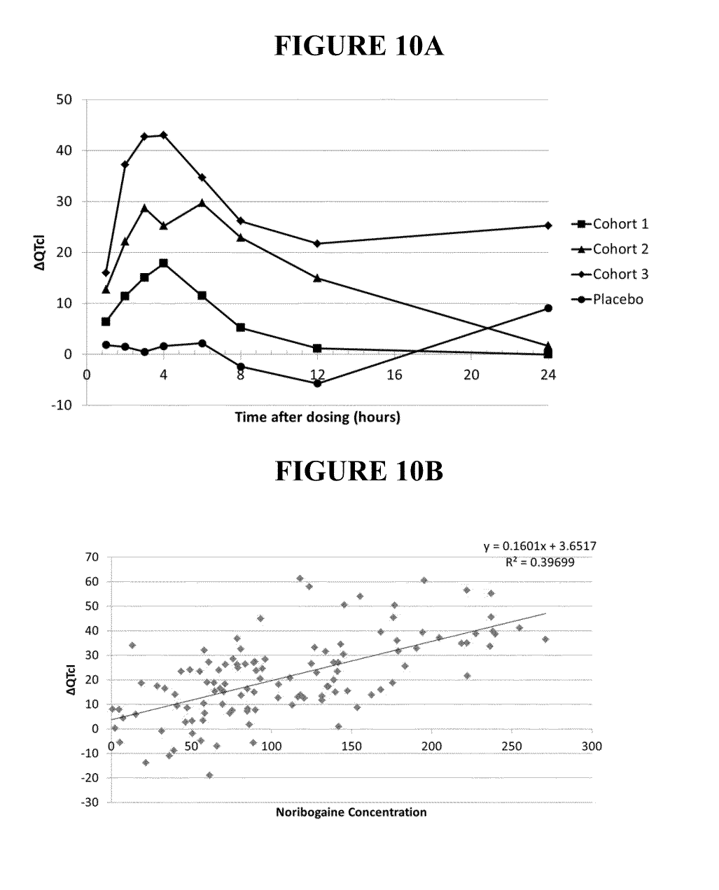

Patients' QT intervals were evaluated at regular time points throughout the study. FIG. 10A shows the average change in QT interval (ΔQTcl, i.e., QT interval prolongation) over the first 24 hours post noribogaine (or placebo) administration. FIG. 10B shows the estimated correlation between noribogaine concentration and change in QT interval. There is a dose-dependent increase in QT interval prolongation that is correlated with the serum concentration of noribogaine.

Based on above data, it is believed that the therapeutic window for a single bolus dose of noribogaine is bound at the lower end by 50 mg and at the upper end by less than 180 mg. In particular, the therapeutic serum concentration in vivo appears to be between about 50 ng/mL and about 180 ng/mL.

### Example 12

### Effect of Noribogaine on Anxiety Disorder in Humans

A male patient, age 45, suffering from generalized anxiety disorder unrelated to the use of any illicit substance, is treated with noribogaine hydrochloride at a dose of about 1 mg/kg/day for a period of four weeks. During the treatment period, the patient's self-reporting of attenuation of at least one of the following symptoms: restlessness or feeling keyed up or on edge, being easily fatigued, difficulty concentrating or mind going blank, irritability, muscle tension, and sleep disturbance are determined.

### Example 13

### Effect of Noribogaine in Zebrafish Expressed by Anxiety-Related Endpoints Animals

A total of 60 adult wild type short-fin zebrafish (˜50:50 male:female ratio) were used in this study. Fish were housed in groups of 20-30 fish per 40-L tank. Tanks were filled with filtered water and maintained at 25° C. Illumination (1000-1100 1×) was provided by ceiling-mounted fluorescent lights on a 12-h cycle (on: 6.00 h, off: 18.00 h) according to the standards of zebrafish care. All fish used in this study were experimentally naïve and fed Tetramin Tropical Flakes (Tetra USA, Blacksburg, Va.) twice a day. Following behavioral testing, the animals were euthanized in 500 mg/L Tricaine (Sigma-Aldrich, St. Louis, Mo.) buffered to pH=7.0. Animal experimentation in this study fully adhered to national and institutional guidelines and regulations.

### Test Compounds

Pilot experiments performed at 1-, 5- and 10-mg/mL revealed sub-maximal efficacy of noribogaine at 1 mg/L, a dose that did not promote any locomotors effects susceptible to be confounded with efficacy endpoints of interest in other protocols, while the 5- and 10-mg/L doses caused reduction in selected swimming motor activity levels. As such, a 1 mg/L dose of noribogaine (DMX1 or Cpd) was chosen. A standard 20-min pre-treatment time was chosen based on experience with a wide range of other neuroactive compounds and the results of pilot studies. This exposure time was also sufficient for provoking physiological (e.g., cortisol and c-fos) responses of zebrafish to multiple drugs. Noribogaine exposure in this study was performed by submerging individual zebrafish in a 1-L plastic beaker for 20 min prior to the testing. The solution was regularly changed after each exposure, to ensure that each fish is exposed to a consistent concentration of noribogaine. Control fish were exposed to noribogaine-free water for the same treatment time, as described above.

### Tests and Procedures

Apparatus: Behavioral testing was performed between 11:00 and 15:00 h using tanks with water adjusted to the holding room temperature (25° C.). The study used the novel tank test (NTT) protocol. NTT represents one of the most commonly used neurophenotyping tests for adult zebrafish. To avoid the test battery or handling effects, each assay was performed once, on a separate individual naïve fish each time. Prior to testing, fish were pre-exposed individually in a 1-L plastic beaker for 20 min to either noribogaine-treated or noribogaine-free water. During testing, zebrafish behavior was recorded by two trained observers blind to the treatments, who manually scored different behavioral endpoints (inter- and intra-rater reliability in all experiments >0.85) with subsequent automated analysis of generated traces by Ethovision XT8.5 software (Noldus IT, Wageningen, Netherlands). The NTT, used to assess zebrafish anxiety and locomotion, was a 1.5-L trapezoidal tank (15 cm height×28 cm top×23 cm bottom×7 cm width; Aquatic Habitats, Apopka, Fla.) maximally filled with water and divided into two equal virtual horizontal portions by a line marking the outside walls. Fish were individually pre-exposed to water (water control) or noribogaine (1, 5 and 10 mg/L) for 20 min and tested in the standard 5-min NTT.

Behavioral analyses: Zebrafish behavior was recorded by trained observers, scoring the latency to reach the top half of the tank(s), time spent in top(s), number of transitions to top, as well as the number and duration(s) of freezing bouts. Freezing was defined as a total absence of movement, except for the gills and eyes, for >2 s. Trials were also recorded to a computer using a USB webcam (2.0-megapixel, Gigaware, UK) and subsequently analyzed by Ethovision XT8.5, assessing distance traveled (m), velocity (m/s), and meandering endpoints. During manual observation, videos were recorded in MPEG1 format with the maximum sample rate 30 fps for each trial by auto-focusing 2.0 MP USB webcams, placed 25 cm in front of the tanks, and attached to laptop computers. Recorded videos were analyzed with Ethovision XT8.5 software. All environments were calibrated for each arena and the calibration axes were placed to designate the origin (0,0) at the center of each tank. The track data for each fish was exported as raw data into separate spreadsheets. The exported traces were independently evaluated on a consensus basis by two trained observers blinded to the treatments, to illustrate the spatial pattern of zebrafish swimming.

Study Design and Data Analysis: The study exposed adult zebrafish individually (15 animals per group) to water control and 3 doses of noribogaine (1, 5 and 10 mg/L) acutely (for 20 min) by water immersion, following testing in NTT for 5 min, prior to euthanizing the fish. Raw data from manual and automatic endpoints were analyzed using GraphPad Prism to generate graphics and descriptive statistics, for manual and computer-generated endpoints. D'Agostino & Pearson omnibus normality K2 test was performed on data groups. When control group passed normality test, data groups were analyzed by the Bennett's test or Boniferroni all paired-wise comparisons test (ANOVA). When data were not following Gaussian distribution or were non-suitable for previously described statistical approach, sub-grouping and/or ranking was performed, data were treated in a differential manner to allow sub-groups and/or categorical comparison. The accepted value for significance was P<0.05 and higher significance was indicated where it applied. For illustration purposes, data analyzed by parametric statistics were represented as mean±SEM, while non-parametric data were represented as scattered points or categorical sub-grouping.

### Results

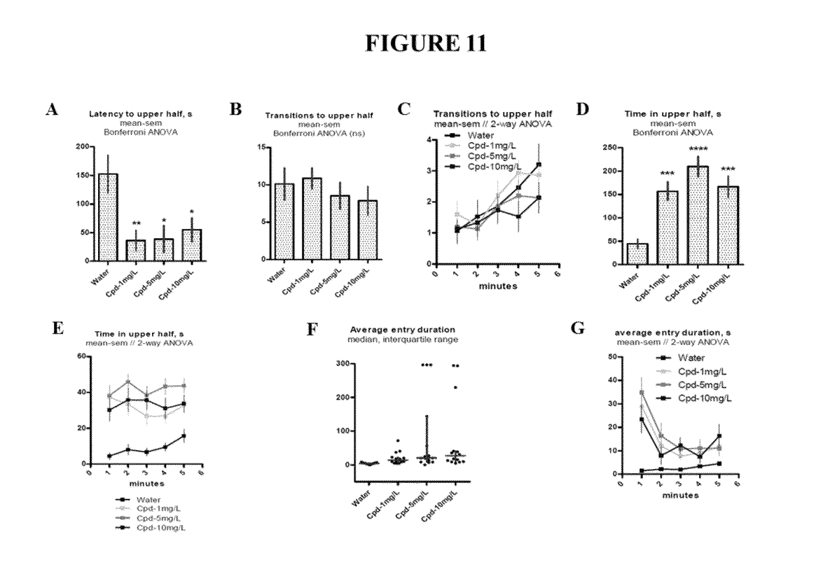

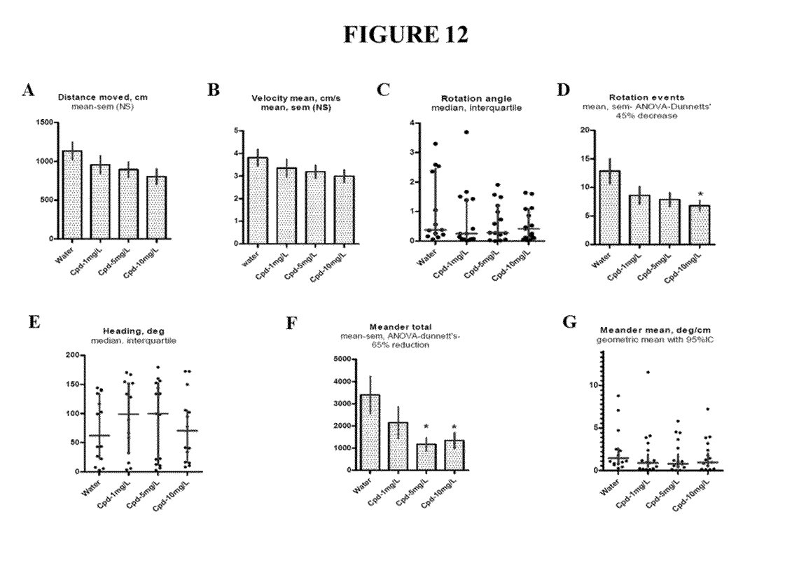

The effects of noribogaine on zebrafish behavior are shown in FIG. 11, panels A-G and FIG. 12, panels A-G. Zebrafish in each of the noribogaine treated groups (1-, 5- and 10-mg/L) showed a statistically significant decrease in latency to the upper half of the tank. FIG. 11, panel A. On the other hand, none of the treatment groups showed a statistically significant change from the control group in transitions to upper half of the tank (FIG. 12, panels B and C), while zebrafish in each treatment group showed a statistically significant increase in the duration of time spent in the defined top portion of the tank (FIG. 12, panels D and E). Zebrafish treated with noribogaine spent on average more time in each of their travels to the top and 2-3 fish in the 5- and 10-mg/L treatment groups spent their entire time in the top portion of the tank (FIG. 12, panel F). The average entry duration in noribogaine-treated zebrafish was higher the first minute and then decrease to steady levels during the 2 to 5 remaining minutes (FIG. 11, panel G).

Total distance traveled in the tank and velocity were measured and zebrafish in the noribogaine-treatment groups exhibited a moderate, but non-significant, decrease in distance moved (FIG. 12, panel A) and velocity (FIG. 12, panel B). In addition, there was a trend of decrease of the high turn angle events for noribogaine-treated zebrafish (FIG. 12, panel C) and a decrease in turning/rotation rate, which was significant at the 10 mg/L dose range (FIG. 12, panel D). Unaltered behavior was observed between the control and noribogaine-treated groups when relative change in direction of body was observed (FIG. 12, panel E). A statistically significant decrease in overall turning rates and patterns (meander) was observed in the 5- and 10-mg/L noribogaine treatment groups (FIG. 12, panel F), but no difference was observed in the meander mean (FIG. 12, panel G).

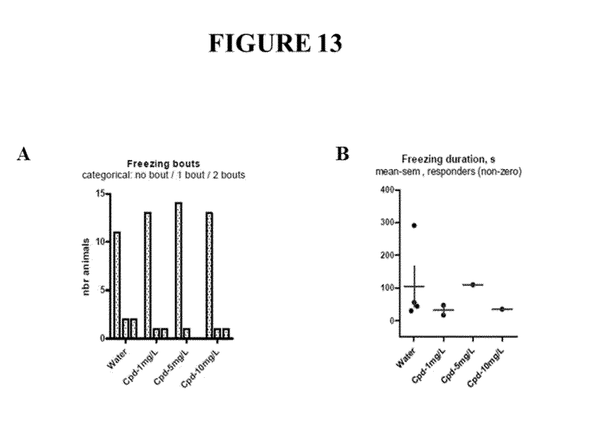

Anxiety/fear responses were also tested by recording freezing bout frequency and freezing duration. FIG. 13. More freezing bouts and longer freezing duration indicate elevated anxiety and/or fear. There was no difference in freezing bouts frequency (number or freezing bouts per 5-minutes) or duration of freezing (FIG. 13, panels A and B).

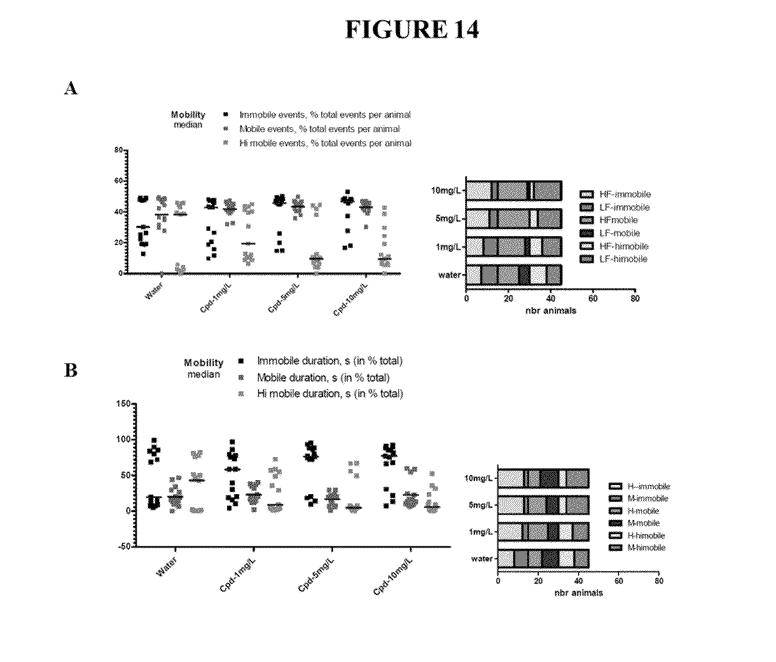

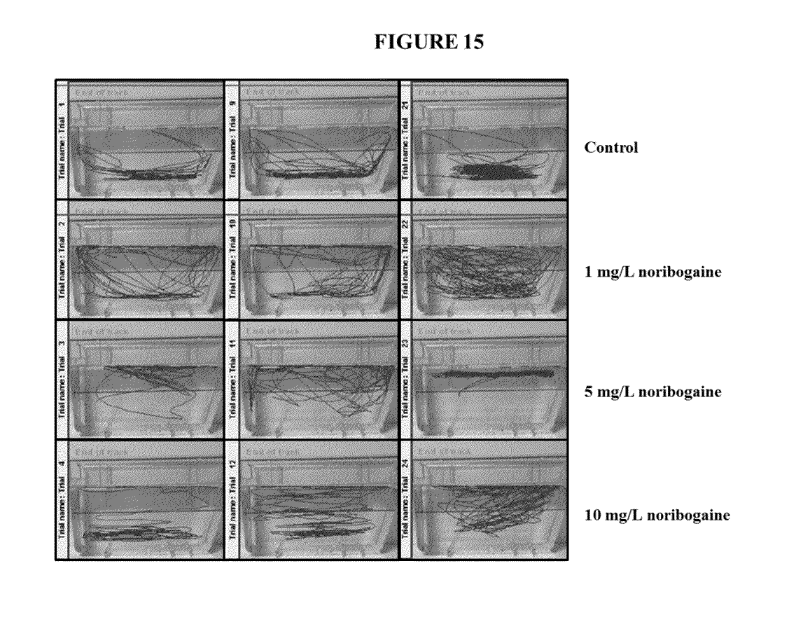

Effect of noribogaine treatment on movement mobility was also detected. FIG. 14. The label “immobile” was used to express the frequency of episodes with degree of movement independent of spatial displacement (duration of immobility). The label “mobile” reflects overall locomotor activity. The label “Hi-mobile” reflects bouts of accelerated swimming (>60% of individual average). Sub-groups were visible with categorical attributes of high frequency (HF) versus low frequency (LF) zebrafish in each cohort (HF/LF threshold at 35%). No difference in average values per subgroup was observed. In addition, there was a dose-related disappearance of HF hi-mobility events with noribogaine treatment and a dose-related disappearance of LF immobile events. There was an overall redistribution to HF-immobile and HF-mobile events (FIG. 14, panel A). Subgroups observed in each cohort were defined as moderate (M) duration versus high (H) duration locomotor activity and no difference in the average values per groups of each sub category (FIG. 14, panel B). The mobile category appeared unaffected. Contingency analysis with moderate (M) versus high (H) duration (threshold 50 sec) per category (immobile, mobile, hi-mobile) indicated a dose related increase in H-immobile category and dose-related decrease of M-himobile category.

### Discussion

Analyses of manual NTT endpoints indicate a statistically significant anxiolytic-like effects of noribogaine at 1, 5 and 10 mg/L, as assessed by shorter latency to enter the top from control (FIG. 11, panel A) and longer time spent by noribogaine-treated fish in the more aversive top (vs. more ‘protective’ bottom) compartment of the test (FIG. 11, panels D and E). In addition, drug-treated zebrafish appeared easier to catch by a net, as noted independently by investigators during the experiments, which indirectly supports reduced anxiety in these cohorts (data not shown). The number of transitions from top to bottom (top entries) did not differ at 1 mg/L-noribogaine treatment group, suggesting the lack of motor impairment (i.e., reflecting unaltered ability to cross the water layers) at this dose (FIG. 11, panels B and C). The two higher doses tended to slightly reduce the number of top transitions from 10 (control) to ˜7 for both doses (FIG. 11, panel B).

Freezing bouts' frequency and duration were unaffected and remained at a generally low level during this study (FIG. 13, panels A and B). This situation is not uncommon for zebrafish NTT studies in general and reflects the specific behavioral features and baseline profile of the zebrafish cohort used. All fish showed normal habituation responses, as assessed by the per-minute distribution of swimming activity in all manual parameters, generally confirming the lack of behavioral anomalies in the applied testing conditions, which were standard and consistent with other published NTT studies. Analyses of computer-generated NTT endpoints reveal a consistent pattern of unaltered motor activity (assessed by distance traveled and velocity measures) at 1 mg/L noribogaine (FIG. 12, panels A and B). Noribogaine slightly reduced total distance traveled at 5 and 10 mg/L in comparison to control group (not significant) (FIG. 12, panels A and B).

Heading (movement directionality index) and mean meandering (straightness index) were similar in all groups, while a significant dose-dependent effect of noribogaine in lowering total meandering can be due to reduced anxiety and/or be due to a relatively slow straighter ‘calm’ swimming in the top part of NTT, as commonly occurs with anxiolytic serotonergic compounds (FIG. 12, panels E-G).

Erratic movements in this study were assessed automatically by measuring the frequency of high-mobility episodes (FIG. 14, panels A and B). These endpoints are generally characteristic of higher anxiety states, but may be seen when reaching characteristic states of altered perception (e.g., hallucinogenic drugs). Fish usually make multiple rotations/turns as part of natural zebrafish anxiety-like arousal behavior, and/or during specific hallucinogenic drug treatments. Noribogaine did not evoke circling behavior (note unaltered turn angle as well), which would have been common for anti-glutaminergic drugs given acutely. On the contrary, noribogaine evoked a statistically significant dose-dependent decrease in the number of turning/rotation rate which could suggest anxiolytic effect, and/or, for example, a direct or indirect stimulation of the NMDA function.

Movement mobility (mobility frequency) and mobility duration, whose endpoints were reflecting general locomotor activity and anxiogenic treatments, showed statistically significant effects of noribogaine treatment (FIG. 14, panels A and B). Subgroups were observed in these endpoints and categorical attributes were assigned to subgroups. Specific redistribution of hi-mobility duration swimming category to the benefit of the immobile duration category with no change of moderate mobility were observed. Because the ‘mobility’ (reflecting normal swimming) remains unaltered, the effect of noribogaine does not appear to be related to general hypolocomotion. However, the redistribution of immobility/high mobility suggests that noribogaine reduced high-velocity accelerated swimming episodes [e.g., typical for anxious fish during erratic ‘high-stress’ swimming, which can be consistent with the observed anxiolytic effect. Furthermore, there was a dose-related disappearance of HF (high-frequency) hi-mobility events with drug treatment and a dose related disappearance of LF (low frequency) immobile events with an overall redistribution to HF-immobile and HF-mobile events (FIG. 14, panel B). The latest variance was issued from a specific subgroup of fish (˜50% of the population) which displayed a moderate total counts of events than the other 50% where high scores of total-counts correlated with higher frequency of immobile events. At 10 mg/L fish showed the lowest ‘high mobility’ high duration contingency (which, again, was highest in more anxious control zebrafish cohort). A similar trend was observed for rotation parameters, especially reduced for 10 mg/L group.

### CONCLUSION

Noribogaine treatment at 1 mg/L evokes a robust anxiolytic-like behavior without any overt hyperactivity/hypoactivity or circling behaviors in zebrafish. Under the condition tested, higher doses of noribogaine were no more efficacious on the relevant and sensitive indices of anxiety levels (time in top, latency in top), indicating that noribogaine half-efficacy in that domain is <1 mg/L. Noribogaine at 5- and 10-mg/mL continued to evoke anxiolytic-like effects, while their behavioral profile in vivo was couple with a reduction of high velocity swim durations shifting to ‘calmer’/slower top swimming activity.

1. A method for treating an anxiety-related disorder in a patient in need thereof, comprising administering to the patient a therapeutically effective amount of noribogaine, noribogaine derivative, or a pharmaceutically acceptable salt and/or solvate thereof, wherein the patient is not addicted to cocaine or an opiate, and further wherein the therapeutically effective amount provides an efficacious average noribogaine serum level of between about 50 ng/mL and about 180 ng/mL while maintaining a QT interval of less than about 500 ms during said treatment.

2. The method of claim 1, wherein the anxiety-related disorder is selected from the group consisting of generalized anxiety disorder, panic disorder, obsessive-compulsive disorder, and social anxiety disorder.

3. A method for treating an impulse control disorder in a patient in need thereof, comprising administering to the patient a therapeutically effective amount of noribogaine, noribogaine derivative, or a pharmaceutically acceptable salt and/or solvate thereof, wherein the patient is not addicted to cocaine or an opiate, and further wherein the therapeutically effective amount provides an efficacious average noribogaine serum level of between about 50 ng/mL and about 180 ng/mL while maintaining a QT interval of less than about 500 ms during said treatment.

4. The method of claim 3, wherein the impulse control disorder is selected from the group consisting of borderline personality disorder, conduct disorder, antisocial personality disorder, attention deficit hyperactivity disorder, attention deficit disorder, schizophrenia, mood disorders, pathological gambling, pyromania, intermittent explosive disorder, kleptomania, sexual compulsion, paraphilia, internet addiction, trichotillomania, pathological skin picking, and compulsive shopping.

5. A method for regulating food intake and/or attenuating food craving in a patient in need thereof, comprising administering to the patient a therapeutically effective amount of noribogaine, noribogaine derivative, or a pharmaceutically acceptable salt and/or solvate thereof, wherein the patient is not addicted to cocaine or an opiate, and further wherein the therapeutically effective amount provides an efficacious average noribogaine serum level of between about 50 ng/mL and about 180 ng/mL while maintaining a QT interval of less than about 500 ms during said treatment.

6. A method for treating an anger-related disorder in a patient in need thereof, comprising administering to the patient a therapeutically effective amount of noribogaine, noribogaine derivative, or a pharmaceutically acceptable salt and/or solvate thereof, and further wherein the therapeutically effective amount provides an efficacious average noribogaine serum level of between about 50 ng/mL and about 180 ng/mL while maintaining a QT interval of less than about 500 ms during said treatment.

7. The method of claim 1, comprising:
a) administering to the patient an initial dose of noribogaine, noribogaine derivative, or pharmaceutically acceptable salt or solvate thereof, wherein the initial dose provides an average serum concentration of about 50 ng/mL to about 180 ng/mL; and
b) administering to the patient at least one additional dose of noribogaine, noribogaine derivative, or pharmaceutically acceptable salt or solvate thereof, such that the at least one additional dose maintains the average serum concentration of about 50 ng/mL to about 180 ng/mL for a period of time.

8. The method of claim 7, wherein the initial dose is from about 75 mg to about 120 mg.

9. The method of claim 7, wherein the at least one additional dose is from about 5 mg to about 25 mg.

10. The method of claim 1, wherein the at least one additional dose is administered from about 6 hours to about 24 hours after the initial dose.

11. The method of claim 1, wherein at least two additional doses are administered, and further wherein the additional doses are administered from about 6 hours to about 24 hours after the previous dose

12. The method of claim 1, wherein the QT interval is less than about 450 ms.

13. The method of claim 1, further comprising selecting a patient who is prescreened to evaluate tolerance for prolongation of QT interval.

14. The method of claim 1, wherein the noribogaine, noribogaine derivative, or pharmaceutically acceptable salt and/or solvate thereof is administered by sublingual, buccal, intranasal, or intrapulmonary delivery.

15. The method of claim 1, wherein noribogaine or a pharmaceutically acceptable salt and/or solvate thereof is administered.

16. The method of claim 1, wherein the noribogaine derivative is represented by Formula I: 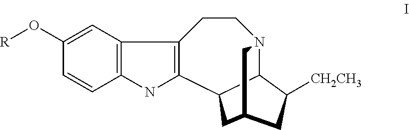
or a pharmaceutically acceptable salt and/or solvate thereof,
wherein R is hydrogen or a hydrolyzable group of the formula: 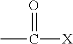
wherein X is an unsubstituted C₁-C₁₂ group or a C₁-C₁₂ group substituted by lower alkyl or lower alkoxy groups, wherein the noribogaine having the hydrolyzable group hydrolyzes in vivo to form 12-hydroxy ibogamine.

17. The method of claim 1, wherein the noribogaine derivative is represented by Formula II: 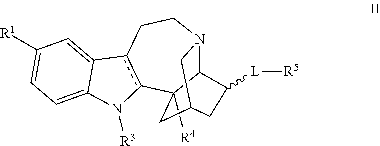
or a pharmaceutically acceptable salt and/or solvate thereof,
wherein [symbol]
is a single or double bond;
R¹ is halo, OR², or C₁-C₁₂ alkyl optionally substituted with 1 to 5 R¹⁰;
R² is hydrogen or a hydrolysable group selected from the group consisting of —C(O) R^{x}, —C(O)OR^{x} and —C(O)N(R^{y})₂ where each R^{x} is selected from the group consisting of C₁-C₆ alkyl optionally substituted with 1 to 5 R¹⁰, and each R^{y} is independently selected from the group consisting of hydrogen, C₁-C₆ alkyl optionally substituted with 1 to 5 R¹⁰, C₆-C₁₄ aryl optionally substituted with 1 to 5 R¹⁰, C₃-C₁₀ cycloalkyl optionally substituted with 1 to 5 R¹⁰, C₁-C₁₀ heteroaryl having 1 to 4 heteroatoms and which is optionally substituted with 1 to 5 R¹⁰, C₁-C₁₀ heterocyclic having 1 to 4 heteroatoms and which is optionally substituted with 1 to 5 R¹⁰, and where each R^{y}, together with the nitrogen atom bound thereto form a C₁-C₆ heterocyclic having 1 to 4 heteroatoms and which is optionally substituted with 1 to 5 R¹⁰ or a C₁-C₆ heteroaryl having 1 to 4 heteroatoms and which is optionally substituted with 1 to 5 R¹⁰;
R³ is selected from the group consisting of hydrogen, C₁-C₁₂ alkyl optionally substituted with 1 to 5 R¹⁰, aryl optionally substituted with 1 to 5 R¹⁰, —C(O)R⁶, —C(O)NR⁶R⁶ and —C(O)OR⁶;
R⁴ is selected from the group consisting of hydrogen, —(CH₂)ₘOR⁸, —CR⁷(OH)R⁸, —(CH₂)ₘCN, —(CH₂)ₘCOR⁸, —(CH₂)ₘCO₂R⁸, —(CH₂)ₘC(O)NR⁷R⁸, —(CH₂)ₘC(O)NR⁷NR⁸R⁸, —(CH₂)ₘC(O)NR⁷NR⁸C(O)R⁹, and —(CH₂)ₘNR⁷R⁸;
m is 0, 1, or 2;
L is a bond or C₁-C₁₂ alkylene;
R⁵ is selected from the group consisting of hydrogen, C₁-C₁₂ alkyl substituted with 1 to 5 R¹⁰, C₁-C₁₂ alkenyl substituted with 1 to 5 R¹⁰, —X¹—R⁷, —(X¹—Y)ₙ—X¹—R⁷, —SO₂NR⁷R⁸, —O—C(O)R⁹, —C(O)OR⁸, —C(O)NR⁷R⁸, —NR⁷R⁸, —NHC(O)R⁹, and —NR⁷C(O)R⁹;
each R⁶ is independently selected from the group consisting of hydrogen, C₁-C₁₂ alkyl, C₂-C₁₂ alkenyl, C₂-C₁₂ alkynyl, C₆-C₁₀ aryl, C₁-C₆ heteroaryl having 1 to 4 heteroatoms, and C₁-C₆ heterocycle having 1 to 4 heteroatoms, and wherein the alkyl, alkenyl, alkynyl, aryl, heteroaryl, and heterocycle are optionally substituted with 1 to 5 R¹⁰;
X¹ is selected from the group consisting of O and S;
Y is C₁-C₄ alkylene or C₆-C₁₀ arylene, or a combination thereof;
n is 1, 2, or 3;
R⁷ and R⁸ are each independently selected from the group consisting of hydrogen, C₁-C₁₂ alkyl optionally substituted with 1 to 5 R¹⁰, C₁-C₆ heterocycle having 1 to 4 heteroatoms and which is optionally substituted with 1 to 5 R¹⁰, C₃-C₁₀ cycloalkyl optionally substituted with 1 to 5 R¹⁰, C₆-C₁₀ aryl optionally substituted with 1 to 5 R¹⁰ and C₁-C₆ heteroaryl having 1 to 4 heteroatoms optionally substituted with 1 to 5 R¹⁰;
R⁹ is selected from the group consisting of C₁-C₁₂ alkyl optionally substituted with 1 to 5 R¹⁰, C₁-C₆ heterocycle having 1 to 4 heteroatoms optionally substituted with 1 to 5 R¹⁰, C₃-C₁₀ cycloalkyl optionally substituted with 1 to 5 R¹⁰, C₆-C₁₀ aryl optionally substituted with 1 to 5 R¹⁰ and C₁-C₆ heteroaryl having 1 to 4 heteroatoms optionally substituted with 1 to 5 R¹⁰;
R¹⁰ is selected from the group consisting of C₁-C₄ alkyl, phenyl, halo, —OR¹¹, —CN, —COR¹¹, —CO₂R¹¹, —C(O)NHR¹¹, —NR¹¹R¹¹, —C(O)NR¹¹R¹¹, —C(O)NHNHR¹¹, —C(O)NR¹¹NHR¹¹, —C(O)NR¹¹NR¹¹R¹¹, —C(O)NHNR¹¹C(O)R¹¹, —C(O)NHNHC(O)R¹¹, —SO₂NR¹¹R¹¹, —C(O)NR¹¹NR¹¹C(O)R¹¹, and —C(O)NR¹¹NHC(O)R¹¹; and
R¹¹ is independently hydrogen or C₁-C₁₂ alkyl;
provided that:
when L is a bond, then R⁵ is not hydrogen;
when [symbol] is a double bond, R¹ is an ester hydrolyzable group, R³ and R⁴ are both hydrogen, then -L-R⁵ is not ethyl;
when [symbol] is a double bond, R¹ is —OH, halo or C₁-C₁₂ alkyl optionally substituted with 1 to 5 R¹⁰, then R⁴ is hydrogen; and
when [symbol] is a double bond, R¹ is OR², R⁴ is hydrogen, -L-R⁵ is ethyl, then R² is not a hydrolyzable group selected from the group consisting of an ester, amide, carbonate and carbamate.

18. The method of claim 1, wherein the noribogaine derivative is represented by Formula III: 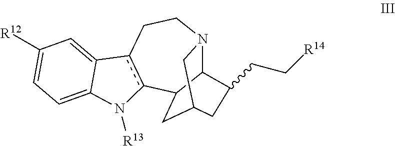
or a pharmaceutically acceptable salt and/or solvate thereof,
wherein [symbol]
is a single or double bond;
R¹² is halo, —OH, —SH, —NH₂, —S(O)₂N(R¹⁷)₂, —R^{z}-L¹-R¹⁸, —R^{z}-L¹-R¹⁹, —R^{z}-L¹-R²⁰ or —R^{z}- L¹-CHR¹⁸R¹⁹, where R^{z} is O, S or NR¹⁷;
L¹ is alkylene, arylene, —C(O)-alkylene, —C(O)-arylene, —C(O)O-arylene, —C(O)O— alkylene, —C(O)NR²⁰-alkylene, —C(O)NR²⁰-arylene, —C(NR²⁰)NR²⁰-alkylene or —C(NR²⁰)NR²⁰-arylene, wherein L¹ is configured such that —O-L¹-R¹⁸ is —OC(O)— alkylene-R¹⁸, —OC(O)O-arylene-R¹⁸, —OC(O)O-alkylene-R¹⁸, —OC(O)-arylene-R¹⁸, —OC(O)NR²⁰-alkylene-R¹⁸, —OC(O)NR²⁰-arylene-R¹⁸, —OC(NR²⁰)NR²⁰-alkylene-R¹⁸ or —OC(NR²⁰)NR²⁰-arylene-R¹⁸, and wherein the alkylene and arylene are optionally substituted with 1 to 2 R¹⁶;
R¹³ is hydrogen, —S(O)₂OR²⁰, —S(O)₂R²⁰, —C(O)R¹⁵, —C(O)NR¹⁵R¹⁵, —C(O)OR¹⁵, C₁-C₁₂ alkyl optionally substituted with 1 to 5 R¹⁶, C₁-C₁₂ alkenyl optionally substituted with 1 to 5 R¹⁶, or aryl optionally substituted with 1 to 5 R¹⁶;
R¹⁴ is hydrogen, halo, —OR¹⁷, —CN, C₁-C₁₂ alkyl, C₁-C₁₂ alkoxy, aryl or aryloxy, where the alkyl, alkoxy, aryl, and aryloxy are optionally substituted with 1 to 5 R¹⁶;
each R¹⁵ is independently selected from the group consisting of hydrogen, C₁-C₁₂ alkyl, C₂-C₁₂ alkenyl, C₂-C₁₂ alkynyl, aryl, heteroaryl, and heterocycle, and wherein the alkyl, alkenyl, alkynyl, aryl, heteroaryl, and heterocycle are optionally substituted with 1 to 5 R¹⁶;
R¹⁶ is selected from the group consisting of phenyl, halo, ˜OR¹⁷, —CN, —COR¹⁷, —CO₂R¹⁷, —NR¹⁷R¹⁷, —NR¹⁷C(O)R¹⁷, —NR¹⁷SO₂R¹⁷, —C(O)NR¹⁷R¹⁷, —C(O)NR¹⁷NR¹⁷R¹⁷, —SO₂NR¹⁷R¹⁷ and —C(O)NR¹⁷NR¹⁷C(O)R¹⁷;
each R¹⁷ is independently hydrogen or C₁-C₁₂ alkyl optionally substituted with from 1 to 3 halo;
R¹⁸ is hydrogen, —C(O)R²⁰, —C(O)OR²⁰, —C(O)N(R²⁰)₂ or —N(R²⁰)C(O)R²⁰;
R¹⁹ is hydrogen, —N(R²⁰)₂, —C(O)N(R²⁰)₂, —C(NR²⁰)N(R²⁰)₂, —C(NSO₂R²⁰)N(R²⁰)₂, —NR²⁰C(O)N(R²⁰)₂, —NR²⁰C(S)N(R²⁰)₂, —NR²⁰C(NR²⁰)N(R²⁰)₂, —NR²⁰C(NSO₂R²⁰)N(R²⁰)₂ or tetrazole; and
each R²⁰ is independently selected from the group consisting of hydrogen, C₁-C₁₂ alkyl and aryl;
provided that:
when [symbol] is a double bond and R¹³ and R¹⁴ are hydrogen, then R¹² is not hydroxy;
when [symbol] is a double bond, R¹⁴ is hydrogen, R¹² is —O-L¹-R¹⁸, —O-L¹-R¹⁹, —O-L¹-R²⁰, and L¹ is alkylene, then —O-L¹-R¹⁸, —O-L¹-R¹⁹, —O-L¹-R²⁰ are not methoxy;
when [symbol] is a double bond, R¹⁴ is hydrogen, R^{z} is O, L¹ is —C(O)-alkylene, —C(O)-arylene, —C(O)O-arylene, —C(O)O-alkylene, —C(O)NR²⁰-alkylene, or —C(O)NR²⁰-arylene, then none of R¹⁸, R¹⁹ or R²⁰ are hydrogen.

19. The method of claim 1, wherein the noribogaine derivative is represented by Formula IV: 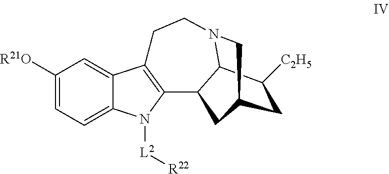
or a pharmaceutically acceptable salt and/or solvate thereof,
wherein
R²¹ is selected from the group consisting of hydrogen, a hydrolysable group selected from the group consisting of —C(O)R²³, —C(O)NR²⁴R²⁵ and —C(O)OR²⁶, where R²³ is selected from the group consisting of hydrogen, alkyl, substituted alkyl, alkenyl, substituted alkenyl, alkynyl and substituted alkynyl, R²⁴ and R²⁵ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic, R²⁶ is selected from the group consisting of alkyl, substituted alkyl, alkenyl, substituted alkenyl, alkynyl, substituted alkynyl, aryl, substituted aryl, heteroaryl, substituted heteroaryl, heterocyclic and substituted heterocyclic, provided that R²¹ is not a saccharide or an oligosaccharide;
L² is selected from the group consisting of a covalent bond and a cleavable linker group;
R²² is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic, provided that R is not a saccharide or an oligosaccharide;
provided that when L² is a covalent bond and R²² is hydrogen, then R²¹ is selected from the group consisting of —C(O)NR²⁴R²⁵ and —C(O)OR²⁶; and
further provided that when R²¹ is hydrogen or —C(O)R²³ and L² is a covalent bond, then R²² is not hydrogen.

20. The method of claim 1, wherein the noribogaine derivative is represented by Formula V: 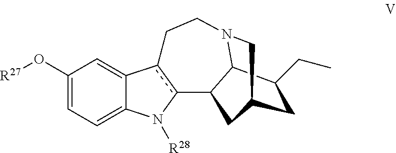
or a pharmaceutically acceptable salt and/or solvate thereof,
wherein: [symbol]
refers to a single or a double bond provided that when [symbol] is a single bond, Formula V refers to the corresponding dihydro compound;
R²⁷ is hydrogen or SO₂OR²⁹;
R²⁸ is hydrogen or SO₂OR²⁹;
R²⁹ is hydrogen or C₁-C₆ alkyl;
provided that at least one of R²⁷ and R²⁸ is not hydrogen.

21. The method of claim 1, wherein the noribogaine derivative is represented by Formula VI: 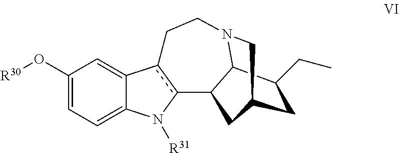
or a pharmaceutically acceptable salt and/or solvate thereof,
wherein: [symbol]
refers to a single or a double bond provided that when [symbol] is a single bond, Formula VI refers to the corresponding vicinal dihydro compound;
R³⁰ is hydrogen, a monophosphate, a diphosphate or a triphosphate; and
R³¹ is hydrogen, a monophosphate, a diphosphate or a triphosphate;
provided that both R³⁰ and R³¹ are not hydrogen;
wherein one or more of the monophosphate, diphosphate and triphosphate groups of R³⁰ and R³¹ are optionally esterified with one or more C₁-C₆ alkyl esters.
